(19) United States
(12) Patent Application Publication
(10) Pub. No.: US 2003/0064372 A1
(43) Pub. Date: Apr. 3, 2003

(54) Gene and sequence variation associated with lipid disorder

(76) Inventors: Jackie S. Bodnar, Los Angeles, CA (US); Lawrence W. Castellani, Sherman Oaks, CA (US); Aurobindo Chatterjee, Ann Arbor, MI (US); Pieter de Jong, Foster City, CA (US); Aldons JAKE Lusis, Los Angeles, CA (US); Jeff Ohmen, Alameda, CA (US); David Ross, Alameda, CA (US); Sherrie Tafuri, Ann Arbor, MI (US); Chenyan Wu, Ann Arbor, MI (US)

Correspondence Address: HOWREY SIMON ARNOLD & WHITE, LLP, BOX 34, 301 RAVENSWOOD AVE., MENLO PARK, CA, 94025, US

(21) Appl. No.: 09/949,428

(22) Filed: Sep. 7, 2001

Related U.S. Application Data
(60) Provisional application No. 60/213,322, filed on Jun. 22, 2000.

(51) Int. Cl. C12Q 1/68; G01N 33/574; C12N 9/16; C07H 21/04; C12N 5/06; C12P 21/02

(52) U.S. Cl. 435/6; 435/7.23; 435/196; 435/320.1; 435/325; 536/23.2; 435/69.1

(57) ABSTRACT

The present invention relates to the discovery of gene and its sequence variation associated with lipid disorder and cancer. The present invention also relates to the study of metabolic pathways and cellular mechanisms to identify other genes, receptors, and relationships that contribute to lipid disorder and cancer. The present invention also relates to germline or somatic sequence variation and its use in the diagnosis and prognosis of predisposition to lipid disorder and cancer. The present invention also provides primers or probes specific for the detection and analysis of such sequence variation. The present invention also relates to methods to screen drugs for inhibition or restoration of gene function as an anti-lipid disorder or anti-cancer therapy. Finally, the present invention relates to other anti-lipid disorder or anti-cancer therapies, such as gene therapy, protein replacement therapy, etc.

This Application claims priority to U.S. Provisional Application Serial No. 60/213,322 filed Sep. 8, 2000.

### FIELD OF THE INVENTION

The present invention relates generally to the field of mouse and human genetics, lipid disorder and cancer. Specifically, the present invention relates to the discovery of a gene and its sequence variation associated with lipid disorder and cancer.

### BACKGROUND OF THE INVENTION

In February 2001, a draft sequence of the human genome was published (International Human Genome Sequencing Consortium, Nature 409:860-921 (2001) and Venter et al., Science, 291:1304-1305 (2001)). This information represents a reference sequence of the 3-billion-base human genome. The remaining task lies in the determination of sequence variations (e.g., mutations, polymorphisms, haplotypes) and sequence functions, which are important for the study, diagnosis, and treatment of human genetic diseases.

An increasing number of genes that play a role in lipid disorders are being identified. Familial combined hyperlipidemia (FCHL) is a common genetic lipid disorder that affects approximately 1-2% of the population in Western societies and accounts for 10-20% of premature coronary heart disease. Increased levels of plasma apolipoprotein B containing lipoproteins, including VLDL and LDL, are observed in FCHL individuals. In addition, they frequently exhibit insulin resistance and tend to have small, dense LDL particles. The aggregate of these abnormalities results in an unfavorable atherogenic risk profile as evidenced by the presence of FCHL in 10-20% of coronary artery disease (CAD) patients under 60 years of age. FCHL is typically characterized by variable expression of both hypertriglyceridemia (triglycerides >90^{th} percentile) and hypercholesterolemia (cholesterol >90^{th} percentile) and a vertical transmission pattern in families (i.e. passed from generation to generation). It appears that most forms of FCHL involve the overproduction of VLDL, but the accumulation of VLDL and its lipolytic products is also influenced by variations in apolipoproteins and lipolytic enzymes. For reviews, see Aouizerat et al., Curr. Opin. Lipidol. 11:113-122 (1999) and de Graaf et al., Curr. Opin. Lipidol 9:189-196 (1998).

Studies have shown that FCHL is complex and heterogeneous. It has been suggested that the FCHL phenotype results from major genes that increase the secretion of VLDL and a number of modifier genes that also influence the levels of plasma lipids. The major genes are likely to be heterogeneous based on the inability to detect strong linkage in preliminary genome scans of Dutch and Finnish pedigrees. One major gene for FCHL was mapped to human chromosome 1q21-q23 in studies of Finnish FCHL families (Pajukanta et al., Nature Genet. 18:369-373 (1998)). Evidence for linkage was found to a locus, adjacent to but separate from the apolipoprotein AII gene on chromosome 1q1-q23. However, major genes in this interval have yet to be identified (Castellani et al., Nat. Genet. 18:374-377 (1998) and Baron et al., Clin. Genet. 57:29-34 (2000)).

Several modifier genes have been reported in various populations, including the lipoprotein lipase (LDL) gene and the apolipoprotein AL-CIII-AIV gene cluster. While these genes are not likely the major genes by linkage analysis, mutations in the LDL gene result in decreased LPL activity in affected individuals (Yang et al., J. Lipid Res. 37:2627-2637 (1996)) and polymorphisms in the apolipoprotein AI genes contribute to the elevated triglyceride levels (Naganawa et al., J. Clin. Invest. 99:1958-1965 (1997)). Recently, several new candidate modifier genes have been reported in Dutch families (Aouizarat et al., Circulation 96(Suppl):545-546 (1997) and in Pima Indians (Celi et al., J Clin Endocrinol Metab 80:2827-2829 (1995)). They include lecithin:cholesterol acyltransferase, manganase superoxide dismutase and fatty acid binding protein 2.

A major difficulty for studies of FCHL relates to the lack of unequivocal diagnostic criteria and the variability of the phenotype, both between affected individuals and over time within one individual. These problems are further compounded by the age-dependence of the hyperlipidemia and environmental influences. To avoid these problems, one important approach is to use animal models that closely resemble the phenotypic features of FCHL. One of the animal models is the HYPLIP1 mutant mouse strain (HcB-19/Dem), which arose as a spontaneous mutation during the development of a recombinant congenic strain between B10 (donor) and C3H (background). The HYPLIP1 mouse exhibits hypertriglyceridemia, hypercholesterolemia, elevated plasma apolipoprotein B, and increased secretion of triglyceride-rich lipoproteins. It also resembles FCHL in other phenotypic features including dramatic age-dependence. Therefore, the HYPLIP1 gene appears to be homologous to one major gene for FCHL.

Considerable effort is also being devoted to constructing mouse models of cancers (Ghebranious et al., Oncogene 17:3385-3400 (1988) and Macleod, J. Pathol. 187:43-60 (1999)). Cancer arises from the abnormal and uncontrolled division of cells that then invade and destroy the surrounding tissues. Two main types of mutations are responsible for cancer. First, gain of function mutations convert normal genes into oncogenes, which act in a dominant fashion and cause malignant tranformation when introduced into normal cells. The non-mutant versions are called proto-oncogenes. The second type of mutation results in the inactivation of both alleles of a suppressor gene. The normal function of such gene is to regulate cell growth in a negative fashion. For reviews, see Lanfrancome et al., Curr. Opin. Genet. Develop. 4:109-119 (1994) and Hinds et al., Curr. Opin. Genet. Develop. 4:135-141 (1994).

In particular, hepatocellular carcinoma (HCC) occurs largely in chronically diseased livers, frequently resulting from hepatitis virus infection, and progression often leads to vascular invasion and intrahepatic metastasis. However, the mechanisms of development and progression of HCC are largely unknown.

### SUMMARY OF THE INVENTION

The present invention provides a gene and its sequence variation associated with lipid disorder and cancer.

The present invention also relates to the study of metabolic pathways and cellular mechanisms to identify other genes, receptors, and relationships that contribute to lipid disorder and cancer.

The present invention also relates to sequence variation and its use in the diagnosis and prognosis of predisposition to lipid disorder and cancer.

The present invention also provides primers and probes specific for the detection and analysis of the HYPLIP1 or FCHL1 locus.

The present invention also relates to kits for detecting a polynucleotide comprising a portion of the HYPLIP1 or FCHL1 locus.

The present invention also relates to a recombinant construct comprising HYPLIP1 or FCHL1 polynucleotide suitable for expression in a transformed host cell.

The present invention also relates to a transgenic animal which carries an altered HYPLIP1 or FCHL1 allele, such as a knockout mouse.

The present invention also relates to methods for screening drugs for inhibition or restoration of FCHL1 gene function as an anti-lipid disorder or anti-cancer therapy.

The present invention also provides therapies directed to lipid disorder or cancer. Therapies of lipid disorder or cancer include gene therapy, protein replacement therapy, protein mimetics, and inhibitors.

More specifically, the present invention provides an isolated polynucleotide comprising a polynucleotide sequence selected from the group consisting of:

(a) a sequence variation of SEQ ID NO: 1, wherein said variation is associated with a lipid disorder or cancer;

(b) a complementary sequence of (a);

(c) a polynucleotide sequence having at least 65% sequence identity to sequence of(a); and

(d) a complementary sequence of (c).

The present invention also provides an isolated polynucleotide comprising a sequence variation of SEQ ID NO: 2 or its complementary sequence, wherein said variation is associated with lipid disorder or cancer.

The present invention also provides an isolated polynucleotide comprising a polynucleotide sequence selected from the group consisting of:

(a) a sequence variation of SEQ ID NO: 4, wherein said variation is associated with lipid disorder or cancer;

(b) a complementary sequence of (a);

(c) a polynucleotide sequence having at least 65% sequence identity to sequence of (a); and

(d) a complementary sequence of (c).

The sequence variations associated with lipid disorder or cancer may be a mutation (e.g., a non-sense mutation) or a polymorphism.

The present invention also provides an isolated polypeptide comprising an amino acid sequence selected from the group consisting of:

(a) a variant form of SEQ ID NO: 3, wherein said variant form is associated with a lipid disorder or cancer; and

(b) an amino acid sequence having at least 65% sequence identity to sequence of (a).

The present invention also provides an isolated polypeptide comprising an amino acid sequence selected from the group consisting of:

(a) a variant form of SEQ ID NO: 5, wherein said variant form is associated with a lipid disorder or cancer; and

(b) an amino acid sequence having at least 65% sequence identity to sequence of (a).

The present invention also provides an isolated polynucleotide having at least 12 contiguous nucleotides spanning the variation position associated with lipid disorder or cancer. The present invention also provides an isolated polypeptide having at least four contiguous amino acids spanning said variant position.

The present invention is also directed to polynucleotides that are specific for the HYPLIP or FCHL1 locus, such as those provided in SEQ ID NO: 6-406. The present invention is also directed to an isolated antibody which is immunoreactive to the polypeptide encoded by the HYPLIP or FCHL1 locus.

The present invention is also directed to a kit for the detection of the HYPLIP or FCHL1 locus and instructions relating to detection.

The present invention also provides a method for analyzing a biomolecule in a sample, wherein said method comprising:

(a) altering HYPLIP1 or FCHL1 activity in a sample; and

(b) measuring the concentration of a biomolecule.

The present invention also provides a method for analyzing a polynucleotide in a sample comprising the steps of:

(a) contacting a polynucleotide in a sample with a probe wherein said probe hybridizes to the polynucleotides of the HYLIP1 or FCHL1 variant to form a hybridization complex; and

(b) detecting the hybridization complex.

The present invention also provides a method for analyzing the expression of HYPLIP1 or FCHL1 comprising the steps of

(a) contacting a sample with a polynucleotide probe; and

(b) detecting the expression of HYPLIP1 or FCHL1 mRNA transcript in said sample.

The present invention also provides a method for identifying susceptibility to a lipid disorder or cancer which comprises comparing the nucleotide sequence of the suspected FCHL1 allele with a wild-type FCHL1 nucleotide sequence, wherein said difference between the suspected allele and the wild-type sequence identifies a sequence variation of FCHL1 nucleotide sequence.

The present invention is also directed to an expression vector or the host cell comprising the polynucleotide of HYPLIP1 or FCHL1 locus.

The present invention is also directed to method for conducting a screening assay to identify a molecule which enhances or decreases the HYPLIP1 or FCHL1 activity comprising the steps of

(a) contacting a sample with a molecule wherein said sample contains HYPLIP1 or FCHL1 activity; and

(b) analyzing the HYPLIP1 or FCHL1 activity in said sample.

The present invention is also directed to a pharmaceutical composition comprising

(a) the polynucleotide of HYPLIP1 or FCHL1 locus, the polypeptide encoded thereby, or the antibody thereof; and

(b) a suitable pharmaceutical carrier.

The present invention is also directed method for treating or preventing a lipid disorder or cancer associated with expression of FCHL1, wherein said method comprising administering to a subject an effective amount of a pharmaceutical composition.

The present invention also provides a transgenic animal which carries an altered HYPLIP1 or FCHL1 allele. In particular, such transgenic animal maybe a knock-out mouse.

### BRIEF DESCRIPTION OF THE FIGURES

FIG. 1. Physical and fine mapping of the HYPLIP1 locus. a, Fine mapping of (HcB-19×CAST/Ei)F2 animals by genotyping 17 microsatellite markers. The ratios of the number of recombinants to the total number of informative mice plus the recombination frequencies±s.e.m. (in cM) are shown. b, The minimum tiling path of the BAC contig for the HYPLIP1 locus. Solid black lines represent 22 individual BAC clones. The BAC clone name is listed, and the BAC size in kb, when known, is given in parenthesis. Markers and BAC end clone sequences are shown at the top, and the estimated physical distances (in kb) are given. The limiting breakpoint markers that define the maximal location of the HYPLIP1 gene are in boldface. c, Four overlapping BACs from the HYPLIP1 locus that were subcloned and sequenced to identify 13 candidate genes. Each BAC clone name is given and the genes are represented as gray boxes with the names listed above in italics. The approximate positions of microsatellite markers and SNPs are shown. The markers that define the maximal location of the HYPLIP1 gene are in boldface type. d, The genomic structure of the HYPLIP1 gene (Vdup1). Solid black lines indicate the eight exons of the Vdup1 gene, and an asterisk indicates the location of the T->A nonsense mutation observed in strain HcB-19. Numbers listed below the figure indicate the DNA base positions of the exon-intron junctions.

FIG. 2. Distributions of triglyceride and ketone body levels. a, Plasma levels of triglycerides in HcB-19 and its C3H parental control. The average value±s.e.m. is shown for six animals in each group. Asterisk indicates a p value <0.0001. b, Distribution of plasma triglyceride values in (HcB-19×CAST/Ei)F2s grouped by genotype at D3Mit101 so that each group represents animals with triglycerides within a certain interval (for example, the group at 30 represents animals with triglycerides from 21-30 mg/dl). Filled bars indicate values for animals homozygous for HcB-19 alleles (h/h), hatched bars indicate heterozygote values (c/h), and open bars denote values for animals homozygous for wildtype CAST/Ei alleles (c/c). The number of animals (N), genotype (Type), and average triglyceride value±s.e.m. (Ave.) in mg/dl for each group are indicated in the legend box. c, Plasma levels of ketone body β-hydroxybutyrate in HcB-19 and its C3H parent control. The average value±s.e.m. is shown for six animals in each group. Asterisk indicates a p value <0.0001. d, Distribution of plasma levels of ketone body β-hydroxybutyrate in (HcB-19×CAST/Ei)F2s grouped by genotype at D3Mit101 so that each group represents animals with plasma ketone body levels within a certain interval (for example, the group at 30 represents animals with ketone bodies from 29-30 mg/dl). Abbreviations and designations are the same as in part b above.

FIG. 3. Recombinant animals and their backcross progeny that define the maximal interval containing the HYPLIP1 gene. Recombinant animals were backcrossed to hyperlipidemic parental strain HcB-19 to generate backcross animals for progeny testing. Backcross mice are grouped according to the inheritance of either recombinant or non-recombinant alleles for the HYPLIP1 region. Triglyceride (TG) and ketone body (Ket.) levels in mg/dl are given for each parental recombinant and their backcross progeny. The predictive probability of being heterozygous, P(c/h), is shown for each parental recombinant and the average predictive probability of being homozygous, P(h/h), is given for backcross progeny that inherited the recombinant chromosome. Filled regions of the chromosome illustrations indicate HcB-19 (h) alleles and open regions indicate CAST/Ei (c) alleles for the DNA markers listed at right. Markers that flank the crossover breakpoint are shown in boldface. a, Recombinant R11 and ten backcross progeny. The parental recombinant and all six backcross progeny that inherited the same haplotype have lower ketone body and triglyceride levels as compared to littermates homozygous for HcB-19 alleles in this region. R11 had a high predictive probability of being heterozygous [P(c/h)=0.987] and the backcross progeny had a low average predictive probability of being homozygous for HYPLIP1 mutant alleles [P(h/h)=0.064]. Since the recombinant chromosome carries CAST/Ei alleles distal to SNP marker D3Pds 7, HYPLIP1 is likely distal to this marker. b, Recombinant R12 and eight backcross progeny. The parental recombinant and all six backcross progeny with the same crossover haplotype have normal ketone bodies and triglycerides, similar to heterozygous littermates, with a low probability of homozygosity for HYPLIP1 mutant alleles [P(h/h)=0. 156]. Thus, HYPLIP1 likely lies proximal to D3Pds13. c, Recombinant R13 and three backcross progeny. As illustrated, R13 carried HYPLIP1 alleles proximal to D3Pds13. Backcross progeny that inherited the crossover have elevated ketone bodies and triglycerides, indicating homozygosity for HYPLIP1 with a high probability [P(h/h)=0.959]. R13 and its backcross progeny yield further evidence that HYPLIP1 is proximal to D3Pds13. d, Recombinant R14, six backcross progeny, and ninety animals obtained from intercrossing the backcross progeny that inherited the crossover breakpoint. The original recombinant R14 had a high predictive probability of being heterozygous [P(c/h)=0.816]. The backcross progeny that inherited the crossover have elevated ketone body and triglyceride levels, indicating homozygosity for HYPLIP1 mutant alleles with a high predictive probability [P(h/h)=0.955]. Furthermore, when these mice were intercrossed to generate animals homozygous for this haplotype, all resultant progeny have elevated ketone bodies and triglycerides (the average±s.e.m. for each group is shown), yielding additional evidence that these animals are homozygous for HYPLIP1 [P(h/h)=0.99], thus placing the distal boundary at D3Pds13.

FIG. 4. Expression and sequence analysis of the Vdup1 gene. a, Northern blot analysis revealing decreased mRNA expression levels for the Vdup1 gene in HcB-19 compared to the C3H control strain. Expression levels for another gene from the HYPLIP1 region, Praja1-L, serves as a RNA loading and locus control. b, Sequence analysis of HcB-19 and C3H mice reveals a T->A transversion mutation present in HcB-19 that is absent from the C3H mice from which it was derived. The sequence chromatograms from HcB-19 and C3H mice are shown, as well as the DNA sequence data from three HcB-19 and three C3H mice. c, Northern blot analysis of the Vdup1 mRNA in various tissues reveals detectable expression in brain, spleen, lung, liver, skeletal muscle, kidney, and testis, with the highest abundance occurring in heart.

FIG. 5. Metabolic consequences of the HYPLIP1 nonsense mutation. a, Total hepatic triglyceride content (in mg per g of liver tissue) from livers of HcB-19 (HcB) and the C3H parental control. Livers were perfused to remove plasma lipids. N=4 C3H animals and 5 HcB-19 animals. Asterisk indicates a p value <0.01. b, Dpm of ¹⁴C-oleic acid per g of liver tissue in newly-synthesized triglycerides secreted from liver slices isolated from fasted HcB-19 and C3H mice. Liver slices were incubated with ¹⁴C-oleic acid in Krebs-Henseleit buffer with 5.5 mM glucose and 3% BSA under 95% 02:5% CO₂. N=6 animals in each group. Asterisk indicates a p value <0.05. c, In vitro secretion of apoB from isolated C3H and HcB-19 hepatocytes as measured by immunoprecipitation after ³⁵S-methionine pulse-labeling. Asterisk indicates a p value <0.05. N=3 animals in each group. d, Plasma free fatty acid levels (in mg/dl) for HcB-19 and C3H. Asterisk indicates a p value <0.01. N=9 animals in each group. e, Amount of newly-synthesized ketone bodies (in dpm per g of liver tissue) from liver slices isolated from HcB-19 or C3H mice and incubated as described above. N=5 C3H animals and 6 HcB-19 animals. Asterisk indicates a p value <0.005. f, Amount of newly-synthesized CO₂ (in dpm per g of liver tissue) from liver slices isolated from fasted HcB-19 and C3H mice and incubated as described above. N=4 C3H animals and 5 HcB-19 animals. Asterisk indicates a p value <0.05. g, Plasma lactate levels (in mg/dl) from HcB-19 and C3H mice. Asterisk indicates a p value <0.001. N=5 animals in each group. h, Pyruvate levels (in mg/dl) from whole blood from HcB-19 and C3H mice. Asterisk indicates a p value <0.008. N=5 animals in each group.

### DETAILED DESCRIPTION OF THE INVENTION

Before the invention is described in detail, it is to be understood that this invention is not limited to the particular component parts or process steps of the method and composition described, as such parts and steps may vary. It is also to be understood that the terminology used herein is for purposes of describing particular embodiments only, and is not intended to be limiting. As used in the specification and the appended claims, the singular forms “a”, “an”, and “the” include plural references.

The present invention provides a gene and its sequence variation associated with lipid disorder and cancer.

The present invention also relates to the study of metabolic pathways and cellular mechanisms to identify other genes, receptors, and relationships that contribute to lipid disorder and cancer.

The present invention also relates to sequence variation and its use in the diagnosis and prognosis of predisposition to lipid disorder and cancer.

The present invention also provides primers and probes specific for the detection and analysis of the HYPLIP1 or FCHL1 locus.

The present invention also relates to kits for detecting a polynucleotide comprising a portion of the HYPLIP1 or FCHL1 locus.

The present invention also relates to a recombinant construct comprising HYPLIP1 or FCHL1 polynucleotide suitable for expression in a transformed host cell.

The present invention also relates to a transgenic animal which carries an altered HYPLIP1 or FCHL1 allele, such as a knockout mouse.

The present invention also relates to methods for screening drugs for inhibition or restoration of FCHL 1 gene function as an anti-lipid disorder or anti-cancer therapy.

Finally, the present invention provides therapies directed to lipid disorder or cancer. Therapies of lipid disorder or cancer include gene therapy, protein replacement therapy, protein mimetics, and inhibitors.

I. Definitions

The present invention employs the following definitions:

As used herein, the term “antibody” refers to polyclonal or monoclonal antibody and fragments thereof, and immunologic binding equivalents thereof. Antibody may be a homogeneous molecular entity, or a mixture such as a serum product made up of a plurality of different molecular entities. Frequently, antibodies are labeled by attaching, either covalently or non-covalently, a substance which provides for a detectable signal, such as radionuclides, enzymes, substrates, cofactors, inhibitors, fluorescent agents, chemiluminescent agents, magnetic particles and the like.

As used herein, the term “antisense” refers to any composition capable of base-paring with the coding stand of a specific nucleic acid sequence. Antisense compositions may include DNA, RNA, peptide nucleic acid, oligonucleotides having modified backbone linkage, for example, phosphorothioates, methylphosphonates, benzylphosphonates, oligonucleotides having modified sugar groups, for example, 2′-methoxy sugars, or oligonucleotides having modified bases, for example, 5-methyl cytosine, 2′-deoxyuracil, or 7-deaza-2′-deoxyguanosine. The designation “negative” or “minus” can refer to the antisense strand, and the designation “positive” or “plus” can refer to the sense strand of a reference polynucleotide.

As used herein, the term “binding partner” refers to a molecule capable of binding another molecule with specificity, as for example, an antigen and an antigen-specific antibody or an enzyme and its inhibitor. Binding partners include, for example, biotin and avidin or streptavidin, IgG and protein A, receptor-ligand couples, protein-protein interaction, and complementary polynucleotide strands.

As used herein, the term “biological sample” refers to a sample derived from a biological source. For example, a biological sample may be derived from a human or animal tissue or fluid, such as plasma, serum, brain, liver, lung, kidney, testis, muscle spleen, heart, muscle, adipose, etc. A biological sample may also be any sample containing a biomolecule.

As used herein, the term “complementary” refers to the relationship between two-stranded polynucleotide sequences that are annealed by base pairing. For example, 5′-TCG-3′ pairs with its complement, 3′-AGC-5.″ Base paring also includes non-Watson-Crick pairs, such as, Hoogsteen pairing.

As used herein, the term “epitope” refers to an antigenic determinant of a polypeptide.

As used herein, the term “homology” refers to sequence identity or sequence similarity between two or more polynucleotide sequences or between two or more polypeptide sequences.

As used herein, the term “hybridization” refers to the process by which a polynucleotide strand anneals with a complementary strand through base pairing under defined hybridization conditions. Specific hybridization is an indication that two nucleic acid sequences share a high degree of complementarity. Specific hybridization complex form under permissive annealing conditions and remain hybridized after the washing step. The washing step is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 g/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. Molecular Cloning: A Laboratory Manual, 3^{rd} Ed., (2000) Cold Spring Harbor Press, Plainview, N.Y.

High stringency conditions for hybridization between polynucleotides include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 g/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The term “hybridization complex” refers to a complex formed between two polynucleotide sequences by the formation of hydrogen bonds between complementary bases. A hybridization complex may be formed in solution or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The terms “percent identity” and “% identity,” as applied to polynucleotide sequences, refer to the percentage of residue matches between at least two polynucleotide sequences aligned using a standardized algorithm. Such an algorithm may insert, in a standardized or reproducible way, gaps in the sequences being compared in order to optimize alignment between two sequences, and therefore achieve a comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, et al., CABIOS 5:151-153 (1989) and in Higgins, et al., CABIOS 8:189-191 (1992). For pairwise alignments of nucleotide sequences, the default parameters may be set as follows: Ktuple=2, gap penalty=5, window=4, and diagonals saved=4. The “weighted” residue weight table maybe selected as the default. Percent identity is reported by CLUSTAL V as the “percent similarity” between aligned polynucleotide sequences.

Other examples of polynucleotide sequence comparison programs include Sequencher™ software available from Gene Codes Corporation (Ann Arbor, Mich.). Alternatively, there are commonly used and freely available sequence comparison algorithms provided by the National Center for Biotechnology Information (NCBI) Basic Logic Alignment Search Tool (BLAST) (Altschul, et al. J. Mol. Biol. 215:403-410 (1990)), which is available from several sources, including the NCBI, Bethesda, Md., and on the internet at http//www.ncbi.n1m.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.n1m.hib.gov/gorf/b12.htm1. The “BLAST 2 Sequences” tool can be used for both blastn and blastp. BLAST programs are commonly used with gap and other parameters set to default settings. For example, to compare two nucleotide sequences, one may use blastn with the ″BLAST 2 Sequences: tool Version 2.0.12 set at default parameters. Such default parameters may be, for example:

| | |
|---|---|
| Matrix: | BLOSUM62 |
| Reward for match: | 1 |
| Penalty for mismatch: | −2 |
| Open Gap: | 5 and Extension Gap: 2 penalties |
| Gap x drop-off: | 50 |
| Expect: | 10 |
| Word Size: | 11 |
| Filter: | on |

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,′ as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail later, generally preserve the charge and hydrophobicity at the site of substitution, thus preserving the structure (and likely function) of the polypeptide.

Percent identity between polypeptide sequences may be determined using the default parameters of the CLUSTAL V algorithm as incorporated into the MEGALIGN version 3.12e sequence alignment program. For pairwise alignments of polypeptide sequences using CLUSTAL V, the default parameters may be set as follows: Ktuple=1, gap penalty=3, windows=5, and “diagonals saved”=5. The PAM250 matrix may be selected as the default residue weight table. As with polynucleotide alignments, the percent identity is reported by CLUSTAL V as the “percent similarity” between aligned polypeptide sequence pairs.

Alternatively, the NCBI BLAST software may be used. For example, for a pairwise comparison of two polypeptide sequences, one may use the “BLAST 2 Sequences” tool Version 2.0.12 with the blastp set at default parameters. Such default parameters may be, for example:

| | |
|---|---|
| Matrix: | BLOSUM62 |
| Open Gap: | 11 and Extension Gap: 1 penalties |
| Gap x drop-off: | 50 |
| Expect: | 10 |
| Word Size: | 3 |
| Filter: | on |

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 10, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

As used herein, the term “polynucleotide” refers to naturally occurring polynucleotide, e.g. DNA or RNA. This term does not refer to a specific length. Thus, this term includes oligonucleotides, primers, probes, genes, regulatory sequences, nucleic acids, etc. This term also refers to analogs of naturally occurring polynucleotides. This term also refers to polynucleotides derived from naturally occurring polynucleotide, such as cDNA. Polynucleotides may be double stranded or single stranded. Polynucleotides may be labeled by attaching, either covalently or non-covalently, a substance which provides for a detectable signal, such as radiolabels, fluorescent labels, enzymatic labels, proteins, haptens, antibodies, sequence tags, etc. Useful labels may include biotin for staining with labeled streptavidin conjugate, magnetic beads (e.g., Dynabeads™), fluorescent molecules (e.g., fluorescein, texas red, rhodamine, green fluorescent protein, FAM, JOE, TAMRA, ROX, HEX, TET, Cy3, C3.5, Cy5, Cy5.5, IRD41, BODIPY and the like), radiolabels (e.g., ³H, ²⁵¹I, ³⁵S, ³⁴S, ¹⁴C ³²P, or ³³P), enzymes (e.g., horse radish peroxidase, alkaline phosphatase and others commonly used in an ELISA), calorimetric labels such as colloidal gold or colored glass or plastic (e.g., polystyrene, polypropylene, latex, etc.) beads, mono and polyfunctional intercalator compounds.

As used herein, the term “polynucleotide amplification” refers to a broad range of techniques for increasing the number of copies of polynucleotide sequences. Typically, amplification of either or both strand of the target nucleic acid comprises the use of one or more nucleic acid-modifying enzymes, such as a DNA polymerase, a ligase, an RNA polymerase, or an RNA-dependent reverse transcriptase. Examples of polynucleotide amplification reaction include, but not limited to, polymerase chain reaction (PCR), nucleic acid sequence based amplification (NASB), self-sustained sequence replication (3SR), strand displacement activation (SDA), ligase chain reaction (LCR), Qβ replicase system, reverse transcriptase PCR (RT-PCR) and the like. For reviews, see Isaksson and Landegren, Curr. Opin. Biotechnol. 10:11-15 (1999), Landegren, Curr. Opin. Biotechnol. 7:95-97 (1996), and Abramson et al., Curr. Opin. Biotechnol. 4:41-47 (1993).

As used herein, the term “primer” refers to a nucleic acid, e.g., synthetic polynucleotide, which is capable of annealing to a complementary template nucleic acid (e.g., the HYPLIP1 or FCHL1 locus) and serving as a point of initiation for template-directed nucleic acid synthesis. A primer need not reflect the exact sequence of the template but should be sufficiently complementary to hybridize with a template. Typically, a primer will include a free hydroxyl group at the 3′ end. The appropriate length of a primer depends on the intended use of the primer but typically ranges from 12 to 40 nucleotides preferably from 15 to 30, most preferably from 18 to 27 nucleotides. The term primer pair (e.g., forward and reverse primers) means a set of primers including a 5′ upstream primer that hybridizes with the 5′ end of the target sequence to be amplified and a 3′, downstream primer that hybridizes with the complement of the 3′ end of the target sequence to be amplified.

As used herein, the term “probe” refers to a polynucleotide of any suitable length which allows specific hybridization to a target sequence. Probes may be may be labeled by attaching, either covalently or non-covalently, a substance which provides for a detectable signal. Typically, probes are at least about 15 nucleotides long, preferably more than at least about 20 or 30 nucleotides long.

As used herein, the term “sequence variation” of a polynucleotide encompasses all forms of polymorphism and mutations. A sequence variation may range from a single nucleotide variation to the insertion, modification, or deletion of more than one nucleotide. A sequence variation may be located at the exon, intron, or regulatory region of a gene.

Polymorphism refers to the occurrence of two or more genetically determined alternative sequences or alleles in a population. A biallelic polymorphism has two forms. A triallelic polymorphism has three forms. A polymorphic site is the locus at which sequence divergence occurs. Diploid organisms may be homozygous or heterozygous for allelic forms. Polymorphic sites have at least two alleles, each occurring at frequency of greater than 1% of a selected population. Polymorphic sites also include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements. The allelic form occurring most frequently in a selected population is sometimes referred to as the wild type form or the consensus sequence.

Mutations include deletions, insertions and point mutations in the coding and noncoding regions. Deletions may be of the entire gene or of only a portion of the gene. Point mutations may result in stop codons, frameshift mutations or amino acid substitutions. Somatic mutations are those which occur only in certain tissues, such as liver, heart, etc and are not inherited in the germline. Germline mutations can be found in any cell of a body and are inherited.

As used herein, the term “target polynucleotide” refers to a single- or double-stranded polynucleotide which is suspected of containing a target sequence, and which may be present in a variety of types of samples, including biological samples. Typically, target sequence is a region of the nucleic acid which is amplified and/or detected. The target polynucleotides may be prepared from human, animal, viral, bacterial, fungal, or plant sources using known methods in the art. For example, target sample may be obtained from an individual being analyzed. For assay of genomic DNA, virtually any biological sample is suitable. For example, convenient tissue samples include whole blood, semen, saliva, tears, urine, fecal material, sweat, buccal, skin and hair. The target polynucleotides may also be obtained from other appropriate source, such as cDNAs, chromosomal DNA, microdissected chromosome bands, cosmid or YAC inserts, and RNA. Target polynucleotides may also be prepared as clones in M13, plasmid or lambda vectors and/or prepared directly from genomic DNA or cDNA.

As used herein, the term “isolated polynucleotide” refers to polynucleotide (e.g., RNA, DNA) which is substantially separated from other cellular components which naturally accompany a native nucleic acid, e.g., proteins, ribosomes, polymerases, and other polynucleotide sequences. In other words, an isolated polynucleotide is removed from its naturally occurring environment. An isolated polynucleotide includes, for example, recombinant or cloned DNA. This term is also known as “substantially pure.”

As used herein, the term “FCHL1 allele” refers to normal alleles of the FCHL1 locus as well as alleles carrying variations that predispose individuals to develop certain type of lipid disorder or cancer. The FCHL1 gene may also refer to as the Vdup1 gene.

As used herein, the term “FCHL1 locus” refers to polynucleotides, which are in the FCHL1 region. The FCHL1 locus includes FCHL1 coding sequences, intervening sequences and regulatory elements controlling transcription and/or translation. The FCHL1 locus includes all allelic variations of the DNA sequence.

As used herein, the term “HYPLIP1 region” refers to a portion of mouse chromosome 3 bounded by the markers P3s11 and Pd167. This region contains the HYPLIP1 locus, including the HYPLIP1 gene.

As used herein, the term “portion” or “fragment” of a polynucleotide refers to a subset of the polynucleotide having a minimal size of at least about 15 contiguous nucleotides, or preferably at least about 20, or more preferably at least about 25 nucleotides.

As used herein, the term “operably linked” refers to a juxtaposition wherein the components are in a relationship permitting them to function in their intended manner. For instance, a promoter is operably linked to a coding sequence if the promoter affects its transcription or expression.

As used herein, the term “regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

As used herein, the term “polypeptide” refers to a polymer of amino acids without referring to a specific length. This term includes naturally occurring protein. The term also refers to modifications, analogues and functional mimetics thereof. For example, modifications of the polypeptide may include glycosylations, acetylations, phosphorylations, and the like. Analogues of polypeptide include unnatural amino acid, substituted linkage, etc. Also included are polypeptides encoded by DNA which hybridize under high or low stringency conditions, to the nucleic acids of interest. Polypeptides may be labeled with radiolabels, fluorescent labels, enzymatic labels, proteins, haptens, antibodies, sequence tags. A polypeptide “fragment,” “portion” or “segment” is a stretch of amino acid residues of at least about five contiguous amino acids, often at least about 10, 15, 20, or 30 contiguous amino acids.

As used herein, the term “proteome” refers to the global pattern of protein expression in a particular tissue, cell line, cell type or other biological sample.

As used herein, the term “isolated polypeptide” refers to a protein or polypeptide which has been separated from components which accompany it in its natural state. A monomeric protein is substantially pure when at least about 60 to 75% of a sample exhibits a single polypeptide sequence. A substantially pure protein typically comprises about 60 to 90% W/W of a protein sample, preferably over about 95%, and more preferably over about 99% pure.

As used herein, the term “FCHL1 polypeptide” refers to a protein or polypeptide encoded by the FCHL1 locus, variants, fragments or functional mimics thereof. The length of FCHL1 polypeptide sequences is generally be at least about 5 amino acids, usually at least about 10, 15, 20, 30 residues. A similar definition applies to “HYPLIP1 polypeptide.”

As used herein, the term “lipid disorder” refers to any disorder that exhibits a phenotypic feature of an increased or decreased level of a biological substance associated with lipid. Biological substances associated with lipid include, for example, lipids, lipoproteins, apoproteins, metabolic intermediate or products, polypeptides associated with lipid (e.g., enzyme using lipid as a substrate), etc. As an example, lipid disorder includes, but not limited to, familial combined hyperlipidemia, coronary artery disease, atherogenic lipoprotein phenotype, hyperapobetalipoproteinemia, hypertriglyceridemia, LDL subclass B, familial dyslipidemic hypertension, syndrome X, hypercholesterolemia, obesity, insulin resistance, etc.

For example, the development of atherosclerosis, the most common form of arteriosclerosis, is correlated with the level of plasma cholesterol. Atherosclerosis begins as intracellular lipid deposits in the smooth muscle cells of the inner arterial wall. These lesions eventually become fibrous, calcified plaques that narrow and even block the arteries. Homozygotes of familial hypercholesterolemia have high levels of the cholesterol-rich LDL in their plasma that their plasma cholesterol levels are three- to fivefold greater than the average level. The rapid formation of atheromas that in homozygotes causes death from myocardial infarction as early as the age of 5. Heterozygotes of familial hypercholesterolemia are less severely afflicted; they develop symptoms of coronary artery disease after the age of 30.

The presence of excess intracellular cholesterol inhibits the synthesis of both LDL receptor and cholesterol. Cells from homozygotes of familiar hypercholesterolemia lack functional LDL receptors, whereas those taken from heterozygotes have about one half of the normal complement. Homozygotes and, to a lesser extent, heterozygotes, are therefore unable to utilize the cholesterol in LDL. These cells must synthesize most of the cholesterol for their needs. The high level of plasma LDL in familiar hypercholesterolemia individuals results from decreased rate of degradation of LDL because of the lack of LDL receptors and increased rate of synthesis from IDL due to the failure of LDL receptors to take up IDL.

As used herein, the term “lipid” refers to a biological substance that is soluble in organic solvent, such as chloroform and are less soluble, if at all, in water. Lipids include substances such as fats, oils, certain vitamins and hormones, and nonprotein membrane components. For example, substances such as fatty acids, fatty acid esters (e.g., triglycerides), fatty (or long chain) alcohols, long chain bases (e.g., sphingoids), glycolipids, phospholipids, sphingolipids, carotenens, polyprenols, sterols (e.g., cholesterol) and related compounds, terpenes, etc, are lipids.

As used herein, the term “fatty acid” refers to a carboxylic acid with long-chain hydrocarbon (e.g., 4 to 24 carbon atoms) side groups. Fatty acids are typically esterified. Fatty acids vary with their degree of unsaturation. They can be saturated (e.g., palmitic acid, stearic acid) or unsaturated fatty acids (e.g., oleic acid, linoleic acid). They can be straight chain or branched acids.

As used herein, the term “triglyceride” (triacylglycerol or neutral fat) refers to a fatty acid triester of glycerol. Triglycerides are typically nonpolar, water-insoluble. Phosphoglycerides (or Glycerophospholipids) are major lipid component of biological membranes. The fats and oils in animals comprise largely mixtures of triglycerides.

As used herein, the term “lipoprotein” refers to any noncovalent association between a protein and lipid. Lipoproteins typically function in the blood plasma as transport vehicles for triglycerides and cholesterol. Plasma lipoproteins form globular particles that comprise a nonpolar core of triglycerides and cholesterol. Lipoproteins include, for example, chylomicrons, very low density lipoproteins (VLDL), intermediate density lipoproteins (IDL), and low density lipoproteins (LDL), high density lipoproteins (HDL). Lipoprotein particles undergo continuous metabolic processing so that they have variable properties and compositions, such as density and particle diameter.

As used herein, the term “apoprotein” (or “apolipoprotein”) refers to protein components of lipoproteins. Apoproteins are typically soluble in water, but tend to aggregate in water. Apoproteins include, but not limited to apoA-II, apoA-II, apoB-48, apoC-I, apoC-II, apoB-100, apoD, apoE, etc.

II. Positional Cloning of Mouse HYPLIP1 Gene and the Discovery of a Gene and its Sequence Variation Associated with Lipid Disorder

An animal model that resembles the phenotypic features of FCHL1 has been developed. HYPLIP1 mutant mouse strain is the result of a spontaneous mutation during the development of a recombinant congenic strain between B10 (donor) and C3H (background). In particular, the Hcb-19 strain exhibits dramatically high triglyceride levels. The Hcb-19 strain also exhibits elevated plasma levels of cholesterol, apolipoprotein B, free fatty acids, ketone bodies, and lactate. The Hcb-19 strain is crossed with the parental strains to examine the mode of inheritance.

Genetic markers are essential for linking a disease to a region of a chromosome. Such markers include restriction fragment length polymorphisms (RFLPS), markers with a variable number of tandem repeats (VNTRS), and polymorphisms based on short tandem repeats (STRs), especially repeats of CpA. To generate a genetic map, one may select potential genetic markers and test them using DNA extracted from animals being studied.

Methods for selecting genetic markers linked with a disease typically include determining the ideal distance between genetic markers of a given degree of polymorphism, then selecting markers from known genetic maps which are ideally spaced for maximal efficiency. The probability that the markers will be heterozygous in unrelated animals is typically measured. Once linkage has been established, one needs to find markers that flank the disease locus, i.e., one or more markers proximal to the disease locus, and one or more markers distal to the disease locus. Where possible, candidate markers can be selected from a known genetic map. Where none is known, new markers can be identified.

Genetic mapping is usually an iterative process. For example, the genetic mapping in the instant invention began by defining flanking genetic markers around the HYPLIP1 locus, then replacing these flanking markers with other markers that were successively closer to the HYPLIP1 locus. Given a genetically defined interval flanked by meiotic recombinants, one needs to generate a contig of genomic clones that spans that interval. For a detailed review of genetic linkage studies, see U.S. Pat. Nos. 5,622,829, 5,709,999, WO00027864, and Ott, J., Analysis of Human Genetic Linkage, The Johns Hopkins University Press, Baltimore and London, 1991.

The present invention provides that a gene, also known as the thioredoxin interaction factor (Tif, see Junn et al., J. Immunol. 164:6287-6295 (2000)), is associated with lipid disorder such as hyperlipidemia and is associated with cancer such as liver cancer. A sequence variation of this HYPLIP1 gene causes reduced expression of the HYPLIP1 gene in the affected mice.

The decoded region of the mouse HYPLIP1 cDNA (SEQ ID NO: 1) is:

| | |
|---|---|
| atggtgatgt tcaagaagat caagtctttt gaggtggtct tcaacgaccc cgagaaggtg | 61 |
| tacggcagcg gggagaaggt ggccggacgg gtaatagtgg aagtgtgtga agttacccga | 121 |
| gtcaaagccg tcaggatcct ggcttgcggc gtggccaagg tcctgtggat gcaagggtct | 181 |
| cagcagtgca aacagacttt ggactacttg cgctatgaag acacacttct cctagaagag | 241 |
| cagcctacag gtgagaacga gatggtgatc atgaggcctg gaaacaaata tgagtacaag | 301 |
| ttcggcttcg agcttcctca agggcccctg ggaacatcct ttaaaggaaa atatggttgc | 361 |
| gtagactact gggtgaaggc ttttctcgat cgccccagcc agccaactca agaggcaaag | 421 |
| aaaaacttcg aagtgatgga tctagtggat gtcaataccc ctgacttaat ggcaccagtg | 481 |
| tctgccaaag aggagaagaa agtttcctgc atgttcattc gtgatggacg tgtgtcagtc | 541 |
| tctgctcgaa ttgacagaaa aggattctgt gaaggtgatg acatctccat ccatgctgac | 601 |
| tttgagaaca cgtgttcccg aatcgtggtc cccaaagcgg ctattgtggc ccgacacact | 661 |
| taccttgcca atggccagac caaagtgttc actcagaagc tgtcctcagt cagaggcaat | 721 |
| cacattatct cagggacttg cgcatcgtgg cgtggcaaga gcctcagagt gcagaagatc | 781 |
| agaccatcca tcctgggctg caacatcctc aaagtcgaat actccttgct gatctacgtc | 841 |
| agtgtccctg gctccaagaa agtcatcctt gatctgcccc tagtgattgg cagcaggtct | 901 |
| ggtctgagca gccggacatc cagcatggcc agccggacga gctctgagat gagctggata | 961 |
| gacctaaaca tcccagatac cccagaagct cctccttgct atatggacat cattcctgaa | 1021 |
| gatcacagac tagagagccc caccacccct ctgctggatg atgtggacga ctctcaagac | 1081 |
| agccctatct ttatgtacgc ccctgagttc cagttcatgc ccccacccac ttacactgag | 1141 |
| gtggatccgt gcgtccttaa caacaacaac aacaacaacg tgcag | |

The mouse HYPLIP1 genomic DNA (SEQ ID NO: 2, an alignment of the genomic sequence and cDNA sequence is shown in examples) is:

TTTTTTTTTAAAAAACAGGTTTGAGGTCATCCTTGGCTATTATAGCAAGTTTGGGGCCAG
CCTGGGACACATGCAACCTTGTCCAAAAAAAAAAAAAAGTCTCTTTGAATTCTTTTTTTT
TGGTTTTTCGAGACAGGGTTTCTCTGTATAGTAGTCTGGGTAGTCTCAAATCCCACAGAC
TCATTTCACAACCCCCACCCCTAAACTACCTTCTTAGGGAAAGACAGGAAAGAAGTAGGC
AGATGAAGGAAAAGACATATTTTACAGTGATTAAGAAACCAAGCTGTTTTGCATCCCTAG
CTCTGACTGTCTGCGGGGGCCAGAGGTGAGAGAATAAGGACCTGCAGGCCTGGCTTCACC
TCCTGTGAAGGCTGCACTGCCAGCTTTGGCACCCGGTTGTCTAGAGTAAAACAAACACAG
GACAAACATTCCTGGCTTCCTACTGGCGCTGAGACTGAACTTGCAAGCCTCTGCTCCCCC
TGGGCACAGCTGTCCTTGTCCCTGAACCCACAGCCTCTGCCCTGTTTTTGTTTATAAGAC
TTTTTTTTCTTCCATCCAAGAACTGAGATGGGTACGTGCGTACATGTGCATGTGCGTGTG
AGTGTGCGTGTGTGTGTGTGTGTGTGAGAGAGAGAGAGAGTGAAGAGGGACAAACTGCTA
TGAGAATACCAGGTGAAAGGTTATAAACAATCCACTCCAGGAGGCAGCCAATTCAGAACA
AGCCTTGGCTATAGGCCCAGGAAGCAGCTGCCACTGCCAGAGTTAAACAGATTTTTGGCC
TAACGCAGAACAAACAAGTGGTCTGTGCTGAGCGCCGCAAATTAAGAAACGATAGCCGTG
CAGGGGACAGGACACAGAACTGTCCACAGGTTTTTCCTTAATTAAAGAATTCAATACTCC
ATAGACAACACCGAATAACTATCAGCATTGCCTCCAAGGAGACAGCCCAAGGCAGCACCC
TCTCACCCCTTAGCAGCCTCCCCTCCTTCATCTGACCTCAAGGTTTAAAAACAAGAACTT
TTTTACATTTAAATTTTTTATTTTGGGTGTCTCTGGAGTGACTACTGGAGAGGGGAAGGA
GAGGGGAGGGGGAGAGGGGAGGTCAGAGTTGGTTCTTTTGCTACTGTGTGGGTTCTGCTA
TTGAACTCAGGTTGGCAGGCCTGGCACCATCTCTCTGGTTCTCCTGAAGTCTTATGTAGC
TGGGGCTGAAGAGATGGCTTGGTGGTTGATAGTATCAGAGGAAACGAGTTCGAGTCCCAG
AACCAAAAAACGGCAGCTCACAACTCTTAACTCCACTTCTAAGGCATCGGCGGACACCTG
CAGCAAGCACACAGGTGGTAGAAGAAAATAACAGCCATATACTTACAAAATTTTTAAATC
TTATGTTGCTACATACCACACTATTTAACAACATCGATATATGAACTTTCGGTATATTTT
GATATTTCATACCATCAAGCTAAGGTTTTCTCAGATGCCTGCTACAGGCACTGAGAAACT
GAAGTTAGTGAGCGACCTACCTCCCTTACAGTATTCATAAATACTGTTTATCGTTGGAAA
ACACCTGACGCCTAGTTAGTTAACTTTCTGGAACAAACACACCCTAAGGATCAAGGTGTT
CCTAGGCCTTGGTGTTGTGTATATGTTTTTGAACCGTGTGATGTTCATCTCTGTGCTTGC
TTAAGGTTCAGTTGTAACTTGTTAGCCTTAGGGTGTCAACCCAGTTAGGGCGCGCGGGGG
TGGGGGCTGGGGGTGTTGTTTATGAACAGCGGTGAACAGGCATGCAATCGCTTTTTACTT
CTCCATCTTAATCTCAGGGCTATATCATCTTTATTTTCCTGCGCAAGGAAGGAGATAGAT
AGTCTCCTAATAATTCTGCCCAAATATGGAAGGAGTTTAGGACTCAATGACAAGGCTCCG
GCGCGGGGTGGGGGTGGGGGTGGGAGTGGGTGGGTGGGTGGGGAATAGAGTAGGGGGCGA
AGGGGGAGGGGGTTGCAGGTAATCCTTCACACAAGAGTTTCTTTGCACACTTAAGAGTTA
TTTCTCTAGTCAGCTCCTGAGGCATCTCTCAGCAAGGTTTGCCAGATAACTAAGTGAAAC
TAACACAGCTCCAGCGCCGGTGAAATTGAAACAGGCTTAGGGACATGCATTTCATTTAGT
GAATTTGGAGAGAGGACAGAGGGGGGAAAAGAATGACAGGAACTCGAAAACAAAGTAAGG
AGTGAGGTTCTTTTTCTTCCTTTTTCTTTCTTTCTTTTATTTTATTTTTTTGGTTTGTCC
ACCTCTTGTTTCCTGGAGAAACAAGGACGGGGGAGCCATCAGTGTGAAAGTAAACACCTC
ACAAAGCTGCAGTGAGGAACAAGGGAACATATACAAAATGTTCCCCAACTTCACAGGTAC
ACTGAAGAGATGAGGGGATAAGCAACAGGATGTGGACACTCCCTTACTGCTTCCGCTCCA
GAGAACAGAATAGAATGTAATGGGCGAGGAACAGTAGCAGCACATAGGGGCATGAAATGA
GGGGGAAATGAGGGGAACCCACCAGAGCATTCACCAGAAAGGACTGAAAGCCAGACTTTA
AAATATCTGACAAGTTCTCGTCTGGAGAGACCGCAGCCTTTTATTCTTCAATAGAAGTGC
AATAGGAGCATATCGGGTGGGCTCTTTCTCACTAACACGACTGCACTCTCGCCCTCCGCT
CCATCCTGGAGTATCCTCGGTGCGATGGGATTGTTTTTCACAAGACTTGCGAACTTGTGA
GCCAGGAATAAATGGTCACCTCGAAATGAATTGCGCTGGCTCAGGCGAGTCATGAAATCC
TCTCCTAAGCACATTTTTCTTTCACCTAAAAAAAGAAGGGGGAAAAAAAAAACAAAGCAC
ACACCCAAATAACCCAGCTCCCAAGAGGAGTCCCCTGGATGAGGTTCAGGGTCCCGGGGT
CCCAGCCTCCCGGGGGGAGGGAGGGCACCCGTCGCCCCGGGCCCCGCCCCTCCTGCTGGC
AAGGCTGCGCACCCGAACAACAACCATTTTCCCCGCTAGGAGCACACCGTGTCCACGCGC
CCCGGCGGCCTCGCTGATTGGTTGGAGGCCTGGTAAACAAGGGCCAAGTAGCCAATGGGA
GAACTGTGCACGAGGGCTGCACGAGCCTCCAGGCCAGCACTCGCGTGGAGCGCCAAGCCA
GGCGGCTATATAAGCCGTNTCCGGCAGCCGGTTGACACTCTTCCTCCTCTGGTCTCGGGG
TTTCCAGAGTTTCTCCAGTTGCGGAAGACAGCTGTTATTTTTCTCCTGAAAGCTTTTGGC
ACAGCCGGCAGGCTGAAACTTCCAGGCACCTTTTGGAAAAGTTGTTAGGGTTTGTTTGAA
GCTTTCTTTACATTTTCGTTTGGGTTTTCAAGCCCTGACTTTACGGAGGCGAGCTCTTCG
TTTGCTTTGAAGGGTTCTTAAAGATTTTTTTCCTCTCCGGCTTTCGTTTTTCTTGAACCC
ACTCGGCTCAATCATGGTGATGTTCAAGAAGATCAAGTCTTTTGAGGTGGTCTTCAACGA
CCCCGAGAAGGTGTACGGCAGCGGGGAGAAGGTGGCCGGACGGGTAATAGTGGAAGTGTG
TGAAGTTACCCGAGTCAAAGCCGTCAGGATCCTGGCTTGCGGCGTGGCCAAGGTCCTGTG
GATGCAAGGGTCTCAGCAGTGCAAACAGACTTTGGACTACTTGCGCTATGAAGACACACT
TCTCCTAGAAGAGCAGCCTACAGGTACTGCTCCCAGCAGGACTGATGGTGACTTGGGAGG
TCTGTGGGTCGGGGAGGGCACCACTAAATGTTTCGAGTTGTTCGTTTGAATGGTTTGAAC
TGTTGGTCCCTATATTTTTTTACTTTGTAATTAGCAAGTTTTTCACTACCCTTCACCCCC
CTAGAGTGATTTGAACACTTTCTGAGGTACTGTTTCCTGAAAGTGTTGTCTTAGCTACTA
CTTAAAGATTAATGTATTTGTGGATTTCGCAACTTTCTGTCCAAGAAAGTGCTCTGGGAT
CTTTTCTTCCATAGTGTAAGAGATGAAAGTGGAAGTGAAGTAAGGTAGTCTACTGCCCAG
GCACTCCTCATTGACGCTTTCAAAATGTAACAAGAAGCCTAATGGCCCCTTGTCTTTGTT
TCCCAGCAGGTGAGAACGAGATGGTGATCATGAGGCCTGGAAACAAATATGAGTACAAGT
TCGGCTTCGAGCTTCCTCAAGGGTAGGCATCCACCGTGTGCACCTTGCACTCTTATTTCT
AAGTCTTCCCCCTCCATTGATCTCTTACAGTTCTTAGCCTTAATTTTGGTTCATTGTTTT
GACACAGGCCCCTGGGAACATCCTTTAAAGGAAAATATGGTTGCGTAGACTACTGGGTGA
AGGCTTTTCTCGATCGCCCCAGCCAGCCAACTCAAGAGGCAAAGAAAAACTTCGAAGTGA
TGGATCTAGTGGATGTCAATACCCCTGACCTAATGGTGAGGATTTTTTGTTTTTGTTTTT
AAAAAGGTTTTAAAATTCTTCTTGGTCAGGGATAATAAATTAGATGCATGGGGGTTGAAA
TATCTCAAAACATTATTTCCTTTTACACAGGCACCAGTGTCTGCCAAAAAGGAGAAGAAA
GTTTCCTGCATGTTCATTCCTGATGGACGTGTGTCAGTCTCTGCTCGAATTGACAGAAAA
GGATTCTGTGAAGGTAAAAACATACTGCTTCAAATGCTAGACAGGATAGCCAGAACTGGG
GGTGGGGGGGTTGGGGGTGGTACGGAGAGGGTCGTAGGGTAGAGGCAGAGGAAGTGCTGT
TAACTTGCATGGCTATTCATACTTCCTCATTTTATTTTAACTCTAGGTGATGACATCTCC
ATCCATGCTGACTTTGAGAACACGTGTTCCCGAATCGTGGTCCCCAAAGCGGCTATTGTG
GCCCGACACACTTACCTTGCCAATGGCCAGACCAAAGTGTTCACTCAGAAGCTGTCCTCA
GTCAGAGGCAATCACATTATCTCAGGGACTTGCGCATCGTGGCGTGGCAAGAGCCTCAGA
GTGCAGAAGATCAGACCATCCATCCTGGGCTGCAACATCCTCAAAGTCGAATACTCCTTG
CTGGTGAGTGGGTGAGAAGAGAGACAATTACCTGGTTACAAATTCAGTGCTTTCTGTACT
CAACCCATCTAACAAACTGCCATCCTCCTCTCTAGATCTACGTCAGTGTCCCTGGCTCCA
AGAAAGTCATCCTTGATCTGCCCCTAGTGATTGGCAGCAGGTCTGGTCTGAGCAGCCGGA
CATCCAGCATGGCCAGCCGGACGAGCTCTGAGATGAGCTGGATAGACCTAAACATCCCAG
ATACCCCAGAAGGTAAGCTGCAGCCGGATAGGTTCGAGTTATTTTGATCTGCTTGGGCTT
GTGGAGTTGGGGTGACCTGGCATTTATTTCTTAGTCGGACTTCTGACACCGTTTTCTCTC
TTCAGCTCCTCCTTGCTATATGGACATCATTCCTGAAGATCACAGACTAGAGAGCCCCAC
CACCCCTCTGCTGGACGATGTGGACGACTCTCAAGACAGCCCTATCTTTATGTACGCCCC
TGAGTTCCAGTTCATGCCCCCACCCACTTACACTGAGGTGAGAACTGCTATTCTCACAGG
GTCAACATTTTGTCCTAGGCCTTTTGAAGGAAGGGTTAATGTGGGTTTTCTACTTAACTA
AAAAACCTGAAAATTTCCTCTCTATTCCCCTTCCAGGTGGATCCGTGCGTCCTTAACAAC
AACAACAACAACAACAACGTGCAGTGAGCCTGCAGGAAATGAAGCATCTGTATTAGCGCA
TTTCTTTCTGCCTCTCTGCTTGAACTCCAGTGTTTCAGAGACTCAGTCTCTACAGCGGGG
AACGGGTACACCCCAGCCGCTGACTCCTCAAGATGGGTGGCAATCAGTAGGCGGGTCTCC
GGCTTCAAGTGGTGCAGACCAGTGCCCGCACTGTGGCATAGGAGTGTTTGCTGGGTGGAT
GTCAGAACACTCTTAGAAAAATTGAGACCTGACCACTTTCTCGGATGTTGGAAATGAAGA
ACTTGTTTGTGTTGACTGAGTCAGGGCACTGCTGACCTTCTGGCGTTGTCTTTCCAAGGT
TTTTGTTTTAAAGGGACTTTTAAATTGTCTAAAATATCAGTAGACCATCATCTGTGCCAT
GGGGGACAGAGCCAATTTCAAGTCATGGCCAAAATTTTGTAAGAGGAGTGTTTTTGTGTG
TTTTTTAAAGTCAGTGTTCCTTTTTTATATCTTTACAAAGAAAAGACCTTCCACGGCTGG
TGATCACGCAGCCTGTGAAATTCGGGGCAGCTGCTCCAAGTTGACTTCACCCTGGGAGCA
GTAGTAGCTGTGCCCACTGACGGCCATAAAAGCCATTTTACAGCCAGTTGCACTGTGTTC
TCTTGTAAGCATAATCAGATGGGAGAATCTGTTATTTCCCTGTAACCCCTTGGAATTGAT
TCTAAGGTGATGTTCTTAGCACTTTAGCTTGTCAATTTTGTTTTAGTCTCCGTTATAGAT
GTAAGCTCCACCAGTCTCTTAAGGATTAAGCCCAGTGACTTGGAGGGTGGGGGTTAGGGT
CTCTATCCCTGAACATTGTAGACCCAGGCTGGCCTGAGAGATCCACCTGCCTCTGCCTCC
TGAGTGCTGCGATCAAAGGCCCAGCTTGGTTATTGCTTTTGAGGCTTTCTCCCAACGCAC
AGACTTGTGTAATTCTAACACTAATCCTGTGAAGGGTTGTGGTTGACAGCTGGAGCCTGG
GTGACATTCTACATTGAGATGCCCCAGCACTGATCGGGGCACAGAAGCCCCCAGACCCCA
TTTCCTGTCCAGTGTTGGGAGAAAGTGCTGCTTTCACTGTGGCCTCAGCCCTGGCTCGGA
AGCTCACTAAGCCTTAGCACTTTGTCCTGTGTCAGCTCCACCTGAGAACTGTGCAGCCAG
AATGTCTGCGAGCTGATGGAGGTTTCGGTTTTGTTGTTTTTGTATTTTGTGTATCTTTTT
GTATGATTAAAAACTATATTTTCTACTTATCCAAATATATTTTCACCCCAAAGTGGGGTT
ATCCTTTGTAAAAAAAAATAAAGTTTTTTAATGACAAAAATAAATGTTCTTTTCTTGTCT
ATGAGATACTGGAGAAGTTACTAGAAAGTGTTCCCCTGTCTCAATACTGAAAGCCCGTGG
AGAGAGAAGTCTCTTGACGCTGAGTGACATAACGGCTGGTTTGGCCTCTGTTCAGACGGA
GGAATCCGTAGGGTCTGGTAGTAGAAGCTAATTAACCACGTCCATAGTCAGAAAACTCCT
TCAGGATCAGGCTTGCTCCTGGGACTGAGGATAGCCTTGAACCTCTGGTGCAGCCATCAA
GAGCACGCAGTGTCATGCTCAGGTTTTCATAGTTTGTGTGTGTGAATGCAGGTGGGAATG
TGGTGCTTAGAACCCACCTTGCAAAAGTCAGCTCCACTTTGTGGGACCCTGAGACCAGGA
CCTCAGGCTTCGCAGAAAGCGTCTTTTACTGCTGAGCCATCTCTGAGCCCAGTTCTCTGC
CCTGTTTATGAATTCTTTAAAAATAACTAAGGGGATTTGGAAGGGACAGGGTGAGATTTT
TATTTTTGTTAAATCCAAATGAGCAGCTTTTGTTTACACAAACGCAGGGAGGATGTGGGG
AAAAGGGACTGGGAGATTAATGTGAGGGAAATTAAATGGGTGTTTGCTCAGATGGGAGGC
AGGAAGCAGTCCTGGTGTGCTCCGGTGGATCTGATGTTCCCTAAAGCTCAGCAGACAGTC
CAGAGTGAGAATGGGTTCTGACTGGCAGAGGCCTCAGCCCACCCTACCCCAAAACAGGAT
GACTGGTGGCAATGGAGTTTTTGGTTTGGTTTGAGACAAGTTCAGGCTAGCCTTAACCTG
GAAGCAATCTGGCTCAGCCTCCCGAGCACTGGGGTTAGAAGACCACGGTCTCATTCATCA
CTTGGTTTTTATTGAGAATTCCCCCAATATAAACTTGGTTTATAAGCTGCAAAGAGGAAC
TATTTCAGACTTGGTTTTAGTTACAGGGATTAAATGTTTTAGAAGCAGCTACAGTTTTCT
GTCTTTATAGATTATTGTGTTTTTTGAGACAGGGTTTCTCTGTAGTCCTGCTCTGTAGAT
CAGGCTAACCCTAAACTCAGAGATCCACTTTCCTCTGTCCCCCGAATGCTGGGATTAGCG
TTTACCACCACAGCCTGACTCTTTACAGTTCTCAACGTATAATTAGAATTCAGTGTCTAC
CCTGATTCCTTGGGACCTGTTTTGGAATTTTCTATTTCTTAGAAGGGTATTGATGACTGA
TAAACCATTTCACTGCTAACTGAAGTTATTTTGTTCAGGAAAAAGCTACACACATGAGAA
ACAAAGATGGCAGAATACATCACACCATTCTTTCTGGTTTTTGGTTCATCTAAATGTTTT
TCGTCAAAATGGGTTTTCCATAGCTCTCCACACACCAGTACACTCTCTGAAGCACTGTAT
TAGAAACCAAGGGGAGGCTCGCTGTGGTCATGCACACCTAANNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNCTGTGAACACAGAACTGAACAGAAATTGAAAAAAAAAGAAATCCTTTTC
TGCGCTAGTAATTGATCTTTATCATTCATTCGCTATAGCGCACCTGTCACTTTCCTGCCT
CACTGGCGCACGCCTTTAATCCCAGCACTCGGGAGGCAGAGGCAGGCAGATTTCTAAGGT
CAAGGCCAGCCTGGTCTACAAAGTGAGTTCCAGGACAGCCAGGGCTACACAGAGAAACCC
TGTCTCAAAAAAACAAAAACAAACAAACAAAAAATAAAATAAACAATAAAACATAAATAA
ATAAAAAGAACAATCATTTGTGTCTGTATACCACAGTGCCCAGGAGGTCAGAGGACTTCT

The mouse HYPLIP1 amino acid sequence (SEQ ID NO: 3) is:

MVMFKKIKSFEVVFNDPEKVYGSGEKVAGRVIVEVCEVTRVKAVRILACG
VAKVLWMQGSQQCKQTLDYLRYEDTLLLEEQPTGENEMVIMRPGNKYEYK
FGFELPQGPLGTSFKGKYGCVDYWVKAFLDRPSQPTQEAKKNFEVMDLVD
VNTPDLMAPVSAKEEKKVSCMFIRDGRVSVSARIDRKGFCEGDDISIHAD
FENTCSRIVVPKAAIVARHTYLANGQTKVFTQKLSSVRGNHIISGTCASW
RGKSLRVQKIRPSILGCNILKVEYSLLIYVSVPGSKKVILDLPLVIGSRS
GLSSRTSSMASRTSSEMSWIDLNIPDTPEAPPCYMDIIPEDHRLESPTTP
LLDDVDDSQDSPIFMYAPEFQFMPPPTYTEVDPCVLNNNNNNNVQ

The corresponding human cDNA (the FCHL1 gene, SEQ ID NO: 4), also known as thioredoxin-binding protein-2 or vitamin D₃ up-regulated protein 1 (Vdup1) in Chen et al., Biochim. Biophysica Acta 1219:26-32 (1994), Nishiyama et al., J. Biol. Chem., 274:21645-21650 (1999) and Shioji et al., FEBS Lett. 472:109-113 (2000)) is:

| | |
|---|---|
| gcttagtgta accagcggcg tatatttttt aggcgccttt tcgaaaacct agtagttaat | 61 |
| attcatttgt ttaaatctta ttttattttt aagctcaaac tgcttaagaa taccttaatt | 121 |
| ccttaaagtg aaataatttt ttgcaaaggg gtttcctcga tttggagctt tttttttctt | 181 |
| ccaccgtcat ttctaactct taaaaccaac tcagttccat catggtgatg ttcaagaaga | 241 |
| tcaagtcttt tgaggtggtc tttaacgacc ctgaaaaggt gtacggcagt ggcgagaggg | 301 |
| tggctggccg ggtgatagtg gaggtgtgtg aagttactcg tgtcaaagcc gttaggatcc | 361 |
| tggcttgcgg agtggctaaa gtgctttgga tgcagggatc ccagcagtgc aaacagactt | 421 |
| cggagtacct gcgctatgaa gacacgcttc ttctggaaga ccagccaaca ggtgagaatg | 481 |
| agatggtgat catgagacct ggaaacaaat atgagtacaa gttcggcttt gagcttcctc | 541 |
| aggggcctct gggaacatcc ttcaaaggaa aatatgggtg tgtagactac tgggtgaagg | 601 |
| cttttcttga ccgcccgagc cagccaactc aagagacaaa gaaaaacttt gaagtagtgg | 661 |
| atctggtgga tgtcaatacc cctgatttaa tggcacctgt gtctgctaaa aaagaaaaga | 721 |
| aagtttcctg catgttcatt cctgatgggc gggtgtctgt ctctgctcga attgacagaa | 781 |
| aaggattctg tgaaggtgat gagatttcca tccatgctga ctttgagaat acatgttccc | 841 |
| gaattgtggt ccccaaagct gccattgtgg cccgccacac ttaccttgcc aatggccaga | 901 |
| ccaaggtgct gactcagaag ttgtcatcag tcagaggcaa tcatattatc tcagggacat | 961 |
| gcgcatcatg gcgtggcaag agccttcggg ttcagaagat caggccttct atcctgggct | 1021 |
| gcaacatcct tcgagttgaa tattccttac tgatctatgt tagcgttcct ggatccaaga | 1081 |
| aggtcatcct tgacctgccc ctggtaattg gcagcagatc aggtctaagc agcagaacat | 1141 |
| ccagcatggc cagccgaacc agctctgaga tgagttgggt agatctgaac atccctgata | 1201 |
| ccccagaagc tcctccctgc tatatggatg tcattcctga agatcaccga ttggagagcc | 1261 |
| caacaactcc tctgctagat gacatggatg gctctcaaga cagccctatc tttatgtatg | 1321 |
| cccctgagtt caagttcatg ccaccaccga cttatactga ggtggatccc tgcatcctca | 1381 |
| acaacaatgt gcagtgagca tgtggaagaa aagaagcagc tttacctact tgtttctttt | 1441 |
| tgtctctctt cctggacact cactttttca gagactcaac agtctcgtca atggagtgtg | 1501 |
| ggtccacctt agcctctgac ttcctaatgt aggaggtggt cagcaggcaa tctcctgggc | 1561 |
| cttaaaggat gcggactcat cctcagccag cgcccatgtt gtgatacagg ggtgtttgtt | 1621 |
| ggatgggttt aaaaataact agaaaaactc aggcccatcc attttctcag atctccttga | 1681 |
| aaattgaggc cttttcgata gtttcgggtc aggtaaaaat ggcctcctgg cgtaagcttt | 1741 |
| tcaaggtttt ttggaggctt tttgtaaatt gtgataggaa ctttggacct tgaacttacg | 1801 |
| tatcatgtgg agaagagcca atttaacaaa ctaggaagat gaaaagggaa attgtggcca | 1861 |
| aaactttggg aaaaggaggt tcttaaaatc agtgtttccc ctttgtgcac ttgtagaaaa | 1921 |
| aaaagaaaaa ccttctagag ctgatttgat ggacaatgga gagagctttc cctgtgatta | 1981 |
| taaaaaagga agctagctgc tctacggtca tctttgctta gagtatactt taacctggct | 2041 |
| tttaaagcag tagtaactgc cccaccaaag gtcttaaaag ccatttttgg agcctattgc | 2101 |
| actgtgttct cctactgcaa atattttcat atgggaggat ggttttctct tcatgtaagt | 2161 |
| ccttggaatt gattctaagg tgatgttctt agcactttaa ttcctgtcaa attttttgtt | 2221 |
| ctccccttct gccatcttaa atgtaagctg aaactggtct actgtgtctc tagggttaag | 2281 |
| ccaaaagaca aaaaaaattt tactactttt gagattgccc caatgtacag aattatataa | 2341 |
| ttctaacgct taaatcatgt gaaagggttg ctgctgtcag ccttgcccac tgtgacttca | 2401 |
| aacccaagga ggaactcttg atcaagatgc ccaaccctgt gatcagaacc tccaaatact | 2461 |
| gccatgagaa actagagggc aggtgttcat aaaagccctt tgaaccccct tcctgccctg | 2521 |
| tgttaggaga tagggatatt ggcccctcac tgcagctgcc agcacttggt cagtcactct | 2581 |
| cagccatagc actttgttca ctgtcctgtg tcagagcact gagctccacc cttttctgag | 2641 |
| agttattaca gccagaaagt gtgggctgaa gatggttggt ttcatgtggg ggtattatgt | 2701 |
| accc | |

The translated region of the human cDNA is from position 222 to 1397. The translated amino acid sequence (SEQ ID NO: 5) is:

MVMFKKIKSFEVVFNDPEKVYGSGERVAGRVIVEVCEVTRVKAVRILACG
VAKVLWMQGSQQCKQTSEYLRYEDTLLLEDQPTGENEMVIMRPGNKYEYK
FGFELPQGPLGTSFKGKYGCVDYWVKAFLDRPSQPTQETKKNFEVVDLVD
VNTPDLMAPVSAKKEKKVSCMFIPDGRVSVSARIDRKGFCEGDEISIHAD
FENTCSRIVVPKAAIVARHTYLANGQTKVLTQKLSSVRGNHIISGTCASW
RGKSLRVQKIRPSILGCNILRVEYSLLIYVSVPGSKKVILDLPLVIGSRS
GLSSRTSSMASRTSSEMSWVDLNIPDTPEAPPCYMDVIPEDHRLESPTTP
LLDDMDGSQDSPIFMYAPEFKFMPPPTYTEVDPCILNNNVQ

Thioredoxin (TRX) is a 12 kDa thiol oxido-reductase that plays an important role in many cellular processes, including cell proliferation, apoptosis, signal transduction, and gene regulation (Holmgren, Structure 3:239-243 (1995); Holmgren, Annu. Rev. Biochem. 54:237-271(1985); and Nakamura et al., Annu. Rev. Immunol. 15:351-369 (1997)). TRX catalyzes the reduction of disulfide bonds in multiple substrate proteins and is a major component of the thiol reducing system. The oxidized form of TRX is reduced to a dithiol by NADPH and the flavoprotein TRX reductase (Buchanan et al., Arch. Biochem. Biophys. 314:257-260 (1994) and Holmgren, supra). Thus, the TRX system is composed of TRX, TRX reductase, and NADPH.

Thioredoxin is widely conserved in almost all species from bacteria to higher eukaryotes, and has a variety of biological functions. The classic function of TRX is to act as a hydrogen donor for ribonucleotide reductase, which is essential for DNA synthesis (Reichard, Science 260:1773-1777 (1993)). In Saccharomyces cerevisiae, deletion of both TRX genes prolonged the cell cycle (Muller, J. Biol Chem. 266:9194-9202 (1991)). Targeted disruption of TRX in mice results in early embryonic lethality, and cells derived from pre-implantation embryos fail to grow in culture (Matsui et al., Dev. Biol. 178:179-185 (1996)). Human TRX is identical to adult T cell leukemia-derived factor (ADF), which has been characterized as a growth factor secreted by human T lymphotropic virus 1-transformed (HTLV1) leukemic cell lines (Tagaya et al., EMBO J. 8:757-764 (1989)). TRX is also overexpressed in cells transformed by Epstein-Barr virus (EBV), hepatitis B virus (HBV), and the human papillomavirus (HPV) (Yamanaka et al., Biochem. Biophys. Res. Commun. 271:796-800 (2000)).

TRX exists in nuclear, cytoplasmic, and secreted forms; its multisite location implies its multifunctional roles as a biological regulator. In the cytosol, TRX regulates signal transduction and has cytoprotective effects against oxidative stress (Nakamura et al., 1997, supra and Ichijo et al., Science 275:90-94 (1997)). Cytoplasmic TRX acts as a powerful antioxidant by reducing reactive oxygen species (ROS) and protects against H₂O₂ and TNF-α: induced cytotoxicity (Nakamura et al., Immunol. Lett. 42:75-80 (1994) and Matsuda et al., J. Immunol. 147:3837-3841 (1991)). Oxidized TRX enters the nucleus where it directly modulates the binding of various transcription factors, including TFIIIC, BZLF1, NF-κB, p53, the estrogen receptor, and the glucocorticoid receptor, as well as indirectly regulates AP-1 activity through Ref-1 (Cromlish et al., J. Biol. Chem. 264:18100-18109 (1989); Bannister et al., Oncogene 6:1243-1250 (1991); Matthews et al., Nucleic Acids Res. 20:3821-3830 (1992); Hayashi et al., Nucleic Acids Res. 25:4035-4040 (1997); Makino et al., J. Biol. Chem. 274:3182-3188 (1999); and Hirota et al., Proc. Natl. Acad. Sci. U.S.A. 94:3633-3638 (1997)). Secreted TRX stimulates the proliferation of lymphoid cells, fibroblasts, and a variety of human solid tumor cell lines, including hepatocellular carcinoma (Blum et al., Cytokine 8:6-13 (1996); Nakamura et al., Cancer 69:2091 -2097 (1992); and Gasdaska et al., Cell Growth Differ. 6:1643-1650 (1995)).

Several studies support a role of TRX in cell proliferation and apoptosis. For example, TRX is a physiological inhibitor for apoptosis signal-regulating kinase 1 (ASK-1), a pivotal component in cytokine- and stress-induced apoptosis (Saitoh et al., EMBO J. 17:2596-2606 (1998)). Stable transfection of the human TRX gene increases cell proliferation in breast cancer cells (Gallegos et al., Cancer Res. 56:5765-5770 (1996)). Furthermore, TRX expression is increased in several types of cancers, including primary human lung and colorectal cancer (Grogan et al., Hum Pathol. 31:475-481 (2000)).

From yeast two-hybrid screens to identify thioredoxin-interacting proteins, both human Vdup1 (hVdup1) and murine Vdup1 (mVdup1) were shown to bind to TRX Nishiyama et al., (1999), supra and Junn et al., 2000, supra)). Vdup1 was first identified as vitamin D₃ up-regulated protein 1, since its expression level is increased in HL-60 cells stimulated to differentiate into monocytes/macrophages by 1,25-dihydroxyvitamin D₃ treatment (Chen et al., (1994), supra). Overexpression of mVdup1 was shown to diminish the endogenous reducing activity of mTRX or the activity of hTRX from a cotransfected cDNA by nearly 50% (Junn et al., 2000). Both hVdup1 and mVdup1 interacted with and inhibited only the reduced form of TRX, and both failed to bind a mutant TRX when either of the two redox-active cysteines were mutated to serines, suggesting that Vdup1 interacts with the catalytic center of TRX (Nishiyama et al., 1999, supra; Junn et al., 2000, supra). In addition, residues 134-395 of mVdup1 and 155 to 225 or beyond of hVdup1 were shown to be required for binding and inhibition of TRX Nishiyama et al., 1999, supra and Junn et al., 2000, supra). Furthermore, mVdup1 was shown to compete with other TRX-binding proteins, such as peroxiredoxin and ASK-1.

Murine Vdup1 is 94% identical to hVdup1, and is ubiquitously expressed in various tissues, such as, heart, brain, spleen, lung, liver, muscle, kidney, and testis, with most abundant expression in heart and secondarily in the liver. The mouse gene is about 5.5 kb with 8 exons while the cDNA is about 2.5 kb. The gene is located on 5.5 kb region on chromosome 3 with a consensus site for polyadenylation that is 1.3 kb downstream of gene, defining a large 3′ untranslated region. The functional Vdup1 promoter contains TATA and CCAAT boxes, and transcription is initiated from two major start sites downstream. A repeat element located proximal to the TATA with homology to the upstream stimulation factor, USF, binding site was identified as a potential regulator of Vdup1 gene expression.

The Vdup1 protein is 395 amino acids in length and approximately 46 kDa. As a negative regulator of function and expression of TRX, it has been shown that Vdup1 is a cytoplasmic protein that binds to and inhibits reduced TRX, with amino acids 155-225 required for binding. By inhibiting the function of TRX, Vdup1 plays a role in cell proliferation and oxidative stress by influencing the redox state of the cell. Vdup1 binds to TRX in vitro and in vivo only when TRX is in the reduced and not oxidized state because it requires two redox active cysteine residues of TRX to bind. The ability to reduce proteins, such as insulin, by TRX is inhibited by Vdup1.

Besides being up-regulated by vitamin D₃ treatment, mVdup1 is also induced in response to various stress stimuli such as H₂O₂, heat shock, γ-rays, and UV exposure. TRX modulates the activity of various transcription factors such as AP-1, NF-KB, PEBP2/AML1, TFIIIC, BZLF1, and plays a role in cell proliferation and oxidative stress. Coexpression of Vdup1 and TRX interfere with TRX binding to DNA transcription factors. TRX is an inhibitor of ASK-1, a component in cytokine and stress induced apoptosis. Therefore, by inhibiting TRX activity, Vdup1 functions as an oxidative stress mediator. Furthermore, overgrown (confluency >90%) NIH 3T3 cells also exhibited rapid induction of mVdup1 expression. Although mVdup1 is known to be increased in response to stress stimuli and shown to inhibit thioredoxin, its exact biological function is relatively uncharacterized.

III. The Study of Metabolic Pathway and Cellular Mechanisms

The present invention is useful in the study of metabolic pathways and cellular mechanisms to identify genes, receptors, and relationships that are associated with lipid disorder and cancer. In particular, the function of the HYPLIP1 and FCHL1 sequences has only been previously known to be important in redox regulations (Junn et al., 2000, supra, Chen et al., 1994, supra, Nishiyama et al., (1999), supra and Shioji et al., 2000, supra). The instant invention thus provides sequence and function information for investigating biochemical pathways, especially, the lipid metabolic pathway, signal transduction pathways, to identify genes, receptors, and relationships that contribute to lipid disorder or cancer, especially in humans.

Lipid metabolic pathways include, for example, lipid digestion, absorption, transport, fatty acid oxidation (e.g, fatty acid activation, transport, and various mechanisms of oxidation), ketone bodies, fatty acid biosynthesis and metabolism, cholesterol metabolism (e.g., biosynthesis, transport, and utilization), arachidonate metabolism, phospholipid, and glycolipid metabolism. Many methods of investigating biochemical pathways are known to those skilled in the art (see e.g., The Metabolic Basis of Inherited Disease (5^{th} ed.), Stanbury et al. (Eds), Part 4, McGraw-Hill (1983), Vance et al., (eds.) Biochemistry of Lipids and Membranes, Benjamin/Cummings (1985)). These methods include, for example, biochemical analysis, genotyping analysis, gene expression analysis, toxicology profiling, proteomic analysis, linkage analysis, statistical analysis, dietary and nutritional studies, etc (see e.g., de Bruin, Curr. Opin. Lipidology 9:275-278 (1998), Masucci-Magoulas et al., Science 275:391-394 (1997), Dominiczak Curr. Opin. Lipidology 11:91-92 (2000), Bakker et al., Atheroscerosis 148:17-21 (2000), Norman et al., J. Clin. Invest. 104:619-628 (1999), Bredie et al, Eur. J. Clin. Invest. 27:802-811 (1997) and Allayee et al., J. Lipid Res. 41:245-252 (2000)).

Biochemical analysis typically involves measurement of the concentration or amount of a biological substance associated lipids, oncogenes, tumor suppressor genes, cell cycle regulation or signal transduction pathway, as a result of altering HYPLIP1 or FCHL1 activity at the nucleic acid or protein level using known methods in the art. For example, altering HYPLIP1 or FCHL1 activity may be accomplished by using genetic or biochemical manipulations or by introducing exogenous agent, etc. A biological substance associated lipid includes, for example, triglyceride, cholesterol, lipoproteins, apolipoproteins, metabolic intermediates and products (e.g., ketone bodies), and enzymes of the lipid metabolic pathways, etc. A lipid disorder typically manifests itself in abnormal amounts of these biomolecules. For example, amounts of lipid-associated biomolecules may vary in different tissues or biological fluids, such as heart, liver, plasma, muscle, and adipose, etc. Amounts of biomolecules may also vary according to age, gender, population, body mass, nutrition, environment, or other biological indexes. In addition to measuring the concentration of lipid-associated biomolecules, ratios, logs, rates, or other mathematical relationships among these biomolecules may also be determined to investigate metabolic pathways and cellular mechanisms in relation to lipid disorder and cancer.

For example, cholesterol is a major component of animal plasma membranes. VLDL, IDL and LDL are a group of related particles that transport endogenous triglycerides and cholesterol from the liver to the tissues. The liver synthesizes triglycerides from excess carbohydrates. HDL typically transport endogenous cholesterol from the tissues to the liver. Cells take up cholesterol through receptor-mediated endocytosis of LDL (Goldstein et al., Annu. Rev. Cell Biol. 1:1-39 (1986)). Blood may be drawn from individuals and plasma lipids (e.g., triglycerides, cholesterol, fatty acids), lipoproteins (e.g., LDL, VLDL, HDL), ketone bodies, or apolipoprotein (e.g., apoB concentrations, apoB/LDL cholesterol ratio) may be quantified using known methods in the art, such as chromatography, enzymatic assay, immunoassay, or commercially available kit, etc. For example, antibodies may immunoprecipitate HYPLIP1 or FCHL1 polypeptides from solution as well as react with HYPLIP1 or FCHL1 polypeptides on Western or immunoblots of polyacrylamide gels. Protein-protein interactions may also be studied to identify downstream targets of HYPLIP1 or FCHL1.

The present invention may also be used to investigate cancer development, progression and treatment. For example, the HcB-19 mouse strain may serve as an animal model in the prevention and treatment of cancer, in particular, hepatic cancer. In particular, mutant mouse that is susceptible to liver tumor may be crossed to other mouse models for hepatic carcinoma. Loss of heterozygosity in Vdup1 in human hepatic cancer may also be studied. See, for example, Pinkel et al., Nature Genet. 20:207-211 (1998) and Wu et al., Cancer Res. 54:6484-6488 (1994).

Identifying oncogenes in cancer studies may be provided by animal tumor viruses. Many animal leukemias, lymphomas, and cancers are caused by viruses. Tumor viruses generally fall into three categories, DNA viruses, retroviruses, and acute transforming retroviruses. DNA viruses infect cells lytically and cause tumors by rare anomalous integration into the host cells. DNA viruses include for example, SV40, Adenovirus, Papilloma virus HPV16, Epstein-Barr virus. Retroviruses contain an RNA genome. They replicate via a DNA intermediate by using viral reverse transcriptase. A typical retrovirus consists of three genes, gag, pol, and env. Examples of retroviruses include HTLV-1, HTLV-2, HIV-1, etc. Acute transforming retroviruses are retrovirus particles transform the host cells rapidly and with high efficiency. They include, for example, Rous sarcoma virus, Harvey rat sarcoma virus, Abelson leukemia virus, Simian sarcoma virus, Erythroleukemia virus, Avian sarcoma virus 17, FBJ osteosarcoma, McDonough feline sarcoma virus, Avian myelocytomatosis virus, etc.

Additionally, identifying oncogenes may be performed by cell tranformation assay, such as a NIH-3T3 assay. For example, mouse 3T3 cells are transfected with random fragments of DNA from a human tumor. Any transformed cells (shown by altered growth) may be isolated, and a phage library may be constructed from their DNA. Phages may then be screened for the human-specific Alu repeat to identify those containing human DNA, which may contain oncogenes. Many oncogenes are mutated versions of genes involved in various normal cellular functions, such as secreted growth factors (e.g., SIS), cell surface receptors (e.g., ERBB, FMS), signal transduction (e.g., RAS, ABL), DNA-binding protein (e.g., MYC, JUN), cell cycle components such as cylines, cycline-dependent kinases and inhibitors thereof (e.g., MDM2). Chromosomal translocations may also generate novel chimeric genes. Oncogenes may also be activated by transposition to an active chromatine domain.

Identifying tumor suppressor gene may be accomplished by positional cloning (e.g., retinoblastoma and BRAC1/BRCA2), loss of heterozygosity screening (e.g., CDKN2A), comparative genomic hybridization (e.g., Pinkel et al., supra (1998)), or cell cycle regulation studies. Tumor suppressor genes may be silenced by methylation in addition to deletion or point mutation.

Many receptors/ion channels/transmembrane signaling proteins have been identified, such as acetylcholine, angiotensin, cadherin, EGF-R, Fas, IGF-1 receptor, integrin α/β, insulin receptor, MuSK, PECAM-1, P2Y2, SDF-1α, TNF-R1. Many kinases have also been identified, such as Akt/PKB, ABL, BCR/ABL, CaMkII, CDK5, CSK, ERK 1/2, FAK, Fyn, GCK, GSK-3beta, MEKK1, MEK3, MEK4, IKK α and β, IKKγ/NEMO, IRS-1, JAK1, JAK2, JAK3, JNK-1 (SAPK), MEK 1/2, NIK, PAK1, 2, 3, PDK-1, PDK-2 (ILK), PKA, P13K, p38 (Erk6), p58IPK, PKC alpha, PKC belta, PKC delta, PKC gamma, PKR, Pyk2, Raf1 (C-raf), B-raf, ROCK, Src, S6K. Several protein phophatases have also been identified, such as MLCK PPase and PTEN. In addition, many transcription and translation factors are also known to those skilled in the art, such as ATF4, beta-Catenin, c-Jun, CREB, FKHRLI, IκB, NFkB, p53, SRF, STAT1alpha, STAT2, STATE3, STATE4, STAT5a, STAT5b, STAT6, TCF, eIF2α. Many adhesion-related/adaptor molecules are also known to those skilled in the art, such as α-acinin, ARP2/3, caldesmon, calpain, caveolin-1, cortactin, CrkL, Desmin, F-actin, FADD, Grb2, Paxilin, PIAS, p130cas, RAIDD, Rapsyn, RIP, Shc, SOCS, SOS, Talin, Tension, TANK, Tau, TRADD, TRAF, Vinculin, WASP, Zyxin. Several phopholipases/phosphodiesterases are also known to those skilled in the art, such as PDE, PLCgammal, PL-D. In addition, many GTPase/GAPs have been identified, such as Rac/cdc42, Rap, Rap1-GAP (C3G), Ras, RhoA, p190RHoGAP. Of course, G-proteins are known to those skilled in the art, such as Adenyl Cyclase, Gq/11, Gi, Go, and Gs. Finally, many caspases/apoptosis related proteins have been identified. They include Apaf-1, Bad, Bax, Bcl-xL, Bcl-2, BID, Caspase 3, Cytochrome-c, PARP, pro-caspase-2, pro-caspase-8, pro-caspase-9, and TERT.

Genotyping of sequence variations of HYPLIP1 or FCHL1 locus may be performed using a variety of methods known to those skilled in the art. These methods include, for example, direct sequencing, array-based hybridization, fluorescent in situ hybridization (FISH), Southern blotting, dot blot analysis, PFGE analysis, single-stranded conformation analysis (SSCA), denaturing gradient gel electrophoresis (DGGE), RNase protection assays, allele-specific oligonucleotides (ASOs), allele-specific PCR, and the use of proteins for recognizing sequence variations, etc.

Direct DNA sequencing, either manual sequencing or automated fluorescent sequencing, is traditionally used to detect sequence variations. The recently developed chip-based hybridization technology is particularly applicable to the present invention. In this high throughput method, hundreds to thousands of polynucleotide probes immobilized on a solid surface are hybridized to nucleic acids of interest to gain sequence information. See, e.g., McKenzie, et al., Eur. J. of Hum. Genet. 6:417-429 (1998), Green et al., Curr.Opin. Chem. Biol. 2:404-410 (1998), and Gerhold et al., TIBS, 24:168-173 (1999). Typically, sets of polynucleotide probes, that differ by having A, T, C, or G substituted at or near the central position, are immobilized on a solid support by in situ synthesis. Fluorescently labeled target nucleic acids containing the expected sequences will hybridize best to perfectly matched polynucleotide probes, whereas sequence variations will alter the hybridization pattern, thereby allowing the determination of mutations and polymorphic sites. See, e.g., Wang, et al., Science 280:1077-1082 (1998) and Lipshutz, et al., Nature Genetics Supplement 21:20-24 (1999), and U.S. Pat. Nos. 5,858,659, 5,856,104, and 6,048,689.

Many indirect sequencing methods are also applicable to the instant invention. SSCA detects a band which migrates differentially because the sequence variation causes a difference in single-strand, intramolecular base pairing. RNase protection involves cleavage of the sequence variation into two or more smaller fragments. DGGE detects differences in migration rates of sequence variants compared to wild-type sequences, using a denaturing gradient gel. In an allele-specific oligonucleotide assay, an oligonucleotide is designed which detects a specific sequence, and the assay is performed by detecting the presence or absence of a hybridization signal. For allele-specific PCR, primers are used which hybridize at their 3′ ends to a particular HYPLIP1 or FCHL1 sequence variation. If the particular HYPLIP1 or FCHL1 sequence variation is not present, an amplification product is not observed. In the mutS assay, the protein binds only to sequences that contain a nucleotide mismatch in a heteroduplex between variant and wild-type sequences. Other approaches based on the detection of mismatches between the two complementary DNA strands include clamped denaturing gel electrophoresis (CDGE), heteroduplex analysis (HA) and chemical mismatch cleavage (CMC). Methods which are more suitable for detecting large sequence variations or detecting a regulatory variation affecting transcription or translation of the protein include protein truncation assay or asymmetric assay. A review of currently available methods of detecting sequence variations can be found in Grompe, Nature Genetics 5:111-117 (1993); Nelson, Crit. Rev. Clin. Lab. Sci. 35:369-414 (1998); Landegren et al., Genome Res. 8:769-776 (1998); and Syvänen, Human Mutation 13:1-10 (1999).

Probes for HYPLIP1 or FCHL1 locus may be derived from the sequences of the HYPLIP1 or FCHL1 region or their cDNAs. The probes may be of any suitable length, which span a portion of the target region, and which allow specific hybridization to the HYPLIP1 or FCHL1 locus. If the target sequence contains a sequence identical to that of the probe, the probes may be short, e.g., in the range of about 8-30 base pairs, since the hybrid will be relatively stable under even highly stringent conditions. If some degree of mismatch is expected with the probe, i.e., if it is suspected that the probe will hybridize to a variant region, a longer probe may be employed which hybridizes to the target sequence with the requisite specificity.

Expression monitoring or profiling analysis may also be performed using the present invention. For example, a mutation in the HYPLIP1 or FCHL1 locus may lead to decreased expression of HYPLIP1 or FCHL1 and may alter the expression of other genes. Point mutations may occur in regulatory regions, such as in the promoter of the gene, leading to loss or reduction of expression of the mRNA. Point mutations may also abolish proper RNA processing, leading to reduction or loss of expression of the HYPLIP1 or FCHL1 gene product, expression of an altered HYPLIP1 or FCHL1 gene product, or to a decrease in mRNA stability or translation efficiency. Mutations that cause disruption to the normal function of the gene product can take a number of forms. The most severe forms may be the frame shift mutations, large deletions or nonsense mutations which would cause the gene to code for an abnormal protein or one which would significantly alter protein expression. Less disruptive mutations may include small in-frame deletions and nonconservative base pair substitutions which would have a significant effect on the protein produced, such as changes to or from a cysteine residue, from a basic to an acidic amino acid or vice versa, from a hydrophobic to hydrophilic amino acid or vice versa, or other mutations which would affect secondary, tertiary or quaternary protein structure. Small deletions or base pair substitutions could also significantly alter protein expression by changing the level of transcription, splice pattern, mRNA stability, or translation efficiency of the HYPLIP1 or FCHL1 transcript. Silent mutations or those resulting in conservative amino acid substitutions would not generally be expected to disrupt protein function.

Many traditional methods of analyzing RNAs are available such as Northern blotting, PCR amplification, RNase protection, in situ hybridization, etc. Monitoring of expression level to compare gene expression patterns using arrays is particularly applicable to the instant invention. For example, many gene-specific polynucleotide probes derived from the 3′ end of RNA transcripts may be spotted on a solid surface. This array is then probed with fluorescently labeled cDNA representations of RNA pools from sample and control cells. The relative amount of transcript present in the pool is determined by the fluorescent signals generated and the level of gene expression is compared between the sample and the control cells. See, e.g., Lockhart et al., Nature 405:827-836 (2000), Roberts et al., Science 287:873-880 (2000), Hughes et al., Nature Genetics 25:333-337 (2000), Hughes et al., Cell 102:109-126 (2000), Duggan, et al., Nature Genetics Supplement 21:10-14 (1999), DeRisi, et al, Science 278:680-686 (1997), and U.S. Pat. Nos. 5,800,992, 5,871,928, 6,040,138, and 6,197,506.

Another aspect of the invention relates to the use of the polynucleotides of the present invention to generate a transcript image of a tissue or cell type. A transcript image may represent the global pattern of gene expression by a particular tissue or cell type. Global gene expression patterns are analyzed by quantifying the number of expressed genes and their relative abundance under given conditions and at a given time. See, for example, U.S. Pat. No. 5,840,484. Methods are also available to monitor gene expression by detecting hybridization to nucleic acids on a solid support using anti-heteronucleic acid antibodies. See, for example, U.S. Pat. No. 6,232,068.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in in vitro model systems and preclinical evaluation of pharmaceuticals, as well as toxicological testing of industrial and naturally-occurring environmental compounds. Frequently, compounds induce unique gene expression patterns, also known as molecular fingerprints or toxicant signatures, which are indicative of mechanisms of action and toxicity (Nuwaysir, et al. Mol. Carcinog. 24:153-159 (1999); Steiner, et al., Toxicol. Lett. 112-113:467-471 (2000)). For example, if a test compound has a signature similar to that of a compound with known toxicity, it is likely to share those toxic properties. In another embodiment, the present invention may also be used to assess therapeutic index, monitor disease state and identify pathways of drug action. See, for example, U.S. Pat. Nos. 5,965,352, 6,197,517, 6,222,093, and 6,218,122.

In addition to profiling transcription levels using polynucleotide probes, methods are also available to profile proteome pattern by quantifying the number of expressed proteins and their relative abundance under given conditions and at a given time. See, for example, Steiner et al., supra (2000) and U.S. Pat. No. 6,278,794. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension. The proteins may be visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification. A proteomic profile may also be generated using antibodies specific for HYPLIP1 or FCHL1 to quantify the levels of HYPLIP1 or FCHL1 expression by reacting the proteins in the sample with a thiol- or amino-reactive fluorescent compound and detecting the amount of fluorescence bound at a solid support (Lueking, et al. Anal. Biochem. 270:103-111 (1999); Mendoze, et al. Biotechniques 27:778-788 (1999)).

In another embodiment, the toxicity of a test compound may be assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

Antisense polynucleotide sequences are useful in preventing or diminishing the expression of the HYPLIP1 or FCHL1 locus. For example, polynucleotide vectors containing all or a portion of the HYPLIP1 or FCHL1 locus or other sequences from the HYPLIP1 or FCHL1 region may be placed under the control of a promoter in an antisense orientation and introduced into a cell. Expression of such an antisense construct within a cell will interfere with HYPLIP1 or FCHL1 transcription and/or translation and/or replication. See for example, Crooke et al., Annu. Rev. Pharmacol. Toxicol. 36:107-129 (1996) and U.S. Pat. No. 6,001,653.

Linkage analysis and statistical analysis may also be performed using a variety of methods known to those skilled in the art (see e.g., U.S. Pat. Nos. 5,622,829, 5,709,999, WO00027864, and Ott, J., Analysis of Human Genetic Linkage, The Johns Hopkins University Press, Baltimore and London, 1991). In particular, multipoint linkage analysis and computer simulation methods may be employed.

In human genetic studies, genetic isolates are important in providing resources. For example, Finnish and Dutch families that fulfill diagnostic criteria may be used as population resource. In particular, the current Finns are thought to have descended from small founder populations of agricultural settlers. Geographical, linguistic and cultural reasons have hindered the mixing of the Finnish population with neighboring populations. For example, each family fulfilling the diagnostic criteria (e.g. lipid values greater than 90th percentile sex-age-specific values in the population) may be studied.

The present invention may also be used to study the effects of diet and nutrition, e.g., vitamin D, on lipid disorder or cancer.

IV. Preparation of Recombinant or Chemically Synthesized Nucleic Acids:

Vectors. Transformation. Host-Cells

Large amounts of the polynucleotides of the present invention may be produced by replication in a suitable host cell (Ausubel et al., Current Protocols in Molecular Biology, Vol. 1-2, John Wiley & Sons (1992) and Sambrook et al, Molecular Cloning A Laboratory Manual, 3rd Ed., Cold Springs Harbor Press (2000)). Natural or synthetic polynucleotide fragments coding for a desired fragment will be incorporated into recombinant polynucleotide constructs, usually DNA constructs, capable of introduction into and replication in a prokaryotic or eukaryotic cell. Usually the polynucleotide constructs will be suitable for replication in a unicellular host, such as yeast or bacteria, but may also be intended for introduction to (with and without integration within the genome) cultured mammalian or plant or other eukaryotic cell lines.

The polynucleotides of the present invention may also be produced by chemical synthesis, e.g., by the phosphoramidite method or the triester method, and may be performed on commercial, automated oligonucleotide synthesizers (see, e.g., Protocols for Oligonucleotides and Analogs; Agrawal, S., Ed.; Humana Press: Totowa, N.J. (1993) and Verma et al., Annu. Rev. Biochem. 67:99-134 (1998)). A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide constructs prepared for introduction into a prokaryotic or eukaryotic host may comprise a replication system recognized by the host, including the intended polynucleotide fragment encoding the desired polypeptide, and may preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate.

An appropriate promoter and other necessary vector sequences will be selected so as to be functional in the host. Many useful vectors are known in the art and may be obtained from commercial vendors. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. In addition, the construct may be joined to an amplifiable gene so that multiple copies of the gene may be made. Appropriate enhancers and other expression control sequences are known in the art.

Expression and cloning vectors may contain a selectable marker, a gene encoding a protein necessary for survival or growth of a host cell transformed with the vector. The presence of this gene ensures growth of only those host cells which express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation” or “transfection.” The cells into which have been introduced nucleic acids described above are meant to also include the progeny of such cells.

Large quantities of the nucleic acids and polypeptides of the present invention may be prepared by expressing the HYPLIP1 or FCHL1 nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, or amphibian or avian species, may also be useful for production of polypeptides of the present invention. Propagation of mammalian cells in culture is well known. Examples of commonly used mammalian host cell lines are VERO and HeLa cells, Chinese hamster ovary (CHO) cells, and W138, BHK, and COS cell lines. An example of a commonly used insect cell line is SF9. However, it will be appreciated by the skilled practitioner that other cell lines may be appropriate, e.g. to provide higher expression, desirable glycosylation patterns, or other features.

Clones are selected by using markers depending on the mode of the vector construction. The marker may be on the same or a different DNA molecule, preferably the same DNA molecule. The transformant may be selected, e.g., by resistance to ampicillin, neomycine, tetracycline or other antibiotics. Production of a particular product based on temperature sensitivity may also serve as an appropriate marker. Markers may also include colormetric methods. For example, green fluorescent protein may be employed.

In addition, biologically active fragments of the HYPLIP1 or FCHL1 polypeptides may also be prepared. Significant biological activities include ligand-binding, immunological activity and other biological activities characteristic of HYPLIP1 or FCHL1 polypeptides. Immunological activities include both immunogenic function in a target immune system, as well as sharing of immunological epitopes for binding, serving as either a competitor or substitute antigen for an epitope of the HYPLIP1 or FCHL1 polypeptides. An epitope could comprise three amino acids in a spatial conformation which is unique to the epitope. Generally, an epitope consists of at least five such amino acids, and more usually consists of at least 8-10 such amino acids. Methods of determining the spatial conformation of such amino acids are known in the art.

For immunological purposes, tandem-repeat polypeptide segments may be used as immunogens, thereby producing highly antigenic proteins. Alternatively, such polypeptides will serve as highly efficient competitors for specific binding.

Fusion proteins comprising HYPLIP1 or FCHL1 polypeptides may also be prepared using known methods in the art. Homologous polypeptides may be fusions between two or more HYPLIP1 or FCHL1 polypeptide sequences or between the sequences of HYPLIP1 or FCHL1 and a related protein. Likewise, heterologous fusions may be constructed which would exhibit a combination of properties or activities of the derivative proteins. For example, ligand-binding or other domains may be swapped between different new fusion polypeptides or fragments. Such homologous or heterologous fusion polypeptides may display, for example, altered strength or specificity of binding. Fusion partners include immunoglobulins, bacterial β-galactosidase, trpE, protein A, β-lactamase, α-amylase, alcohol dehydrogenase and yeast alpha mating factor.

Fusion proteins will typically be made by either recombinant nucleic acid methods or may be chemically synthesized. Techniques for the synthesis of polypeptides are known in the art.

Functional mimetics of a native polypeptide may be obtained using known methods in the art. For example, polypeptides may be at least about 65% homologous to the native amino acid sequence, preferably in excess of about 70%, and more preferably at least about 90% homologous. Substitutions typically contain the exchange of one amino acid for another at one or more sites within the polypeptide, and may be designed to modulate one or more properties of the polypeptide, such as stability against proteolytic cleavage, without the loss of other functions or properties. Amino acid substitutions may be made on the basis of similarity in polarity, charge, solubility, hydrophobicity, hydrophilicity, and/or the amphipathic nature of the residues involved. Preferred substitutions are ones which are conservative, that is, one amino acid is replaced with one of similar shape and charge. Conservative substitutions are well known in the art and typically include substitutions that are predicted to least interfere with the properties of the native protein. For example, alanine may be substitued by glycine or serine; arginine by histidine or lysine; asparagine by aspartic acid, glutamine or histidine; aspartic acid by asparagine or glutamic acid; cysteine by alanine or serine; glutamine by asparagine, glutamic acid, or histidine; glutamic acid by aspartic acid, glutamine, or histidine; glycine by alanine; histidine by asparagine, arginine, glutamic acid, or glutamine; isoleucine by leucine or valine; leucine by isoleucine or valine; lysine by arginine, glutamic acid, or glutamine; methionine by leucine or isoleucine; phenylalanine by histidine, methionine, leucine, trptophan, or tyrosine; serine by cysteine or threonine; threonine by serine or valine; trptophan by phenylalanine or tyrosine; tyrosine by histidine, phenylalanine or trptophan, valine by isoleucine, leucine or threonine.

Certain amino acids may be substituted for other amino acids in a polypeptide structure without appreciable loss of interactive binding capacity with structures such as, for example, antigen-binding regions of antibodies or binding sites on substrate molecules or binding sites on proteins interacting with a polypeptide. Since it is the interactive capacity and nature of a polypeptide which defines that polypeptide's biological functional activity, certain amino acid substitutions can be made in a protein sequence, and its underlying DNA coding sequence, and nevertheless obtain a protein with like properties. In making such changes, the hydropathic index of amino acids may be considered. The importance of the hydrophobic amino acid index in conferring interactive biological function on a protein is generally understood in the art. Alternatively, the substitution of like amino acids can be made effectively on the basis of hydrophilicity.

A peptide mimetic may be a peptide-containing molecule that mimics elements of protein secondary structure. The underlying rationale behind the use of peptide mimetics is that the peptide backbone of proteins exists mainly to orient amino acid side chains in such a way as to facilitate molecular interactions, such as those of antibody and antigen, enzyme and substrate or scaffolding proteins. A peptide mimetic is designed to permit molecular interactions similar to the natural molecule. A mimetic may not be a peptide at all, but it will retain the essential biological activity of a natural polypeptide.

Polypeptides may be produced by expression in a prokaryotic cell or produced synthetically. These polypeptides typically lack native post-translational processing, such as lipiadtion, phosphorylation, acetylation, racemization, proteolytic cleavage, glycosylation.

V. Diagnosis or Screening

Genetic analysis of human diseases is often complicated by the lack of a simple diagnostic mark. For example, currently there is no single diagnostic marker for the diagnosis of familial combined hyperlipidemia available and there are little known targets for hepatic tumor. Sequence variation of the FCHL1 locus may indicate a predisposition to lipid disorder or cancer and may provide a diagnostic mark.

In order to detect the presence of a FCHL1 allele predisposing an individual to a condition, a biological sample may be prepared and analyzed for the presence or absence of susceptibility alleles of FCHL1. Results of these tests and interpretive information may be returned to the health care professionals for communication to the tested individual. Such diagnoses may be performed by diagnostic laboratories. In addition, diagnostic kits may be manufactured and available to health care providers or to private individuals for self-diagnosis.

A basic format for sequence or expression analysis is finding sequences in DNA or RNA extracted from affected family members which create abnormal FCHL1 gene products or abnormal levels of FCHL1 gene product. The diagnostic or screening method may involve amplification or molecular cloning of the relevant FCHL1 sequences. For example, PCR based amplification may be used. Once amplified, the resulting nucleic acid can be sequenced or used as a substrate for DNA probes. Primers and probes specific for the FCHL1 gene sequences may be used to identify FCHL1 alleles.

The pairs of single-stranded DNA primers can be annealed to sequences within or surrounding the FCHL1 gene in order to prime amplifying DNA synthesis of the FCHL1 gene. The set of primers may allow synthesis of both intron and exon sequences. Allele-specific primers can also be used. Such primers anneal only to particular FCHL1 mutant alleles, and thus will only amplify a product in the presence of the mutant allele as a template.

In order to facilitate subsequent cloning of amplified sequences, primers may have restriction enzyme site sequences appended to their 5′ ends. Thus, all nucleotides of the primers are derived from FCHL1 sequences or sequences adjacent to FCHL1, except for the few nucleotides necessary to form a restriction enzyme site. Such enzymes and sites are well known in the art. The primers themselves can be synthesized using techniques which are well known in the art. Generally, the primers can be made using oligonucleotide synthesizers which are commercially available.

The biological sample to be analyzed, such as blood, may be treated, if desired, to extract the nucleic acids. The sample nucleic acid may be prepared in various ways to facilitate detection of the target sequence; e.g. denaturation, restriction digestion, electrophoresis or dot blotting. The region of interest of the target nucleic acid is usually at least partially single-stranded to form hybrids with the probe. If the sequence is double-stranded, the sequence will probably need to be denatured. The target nucleic acid may be also be fragmented to reduce or eliminate the formation of secondary structures. The fragmentation may be performed using a number of methods, including enzymatic, chemical, thermal cleavage or degradation. For example, fragmentation may be accomplished by heat/Mg²⁺treatment, endonuclease (e.g., DNAase 1) treatment, restriction enzyme digestion, shearing (e.g., by ultrasound) or NaOH treatment.

Many genotyping and expression monitoring methods have been described previously. In general, target nucleic acid and probe are incubated under conditions which forms a hybridization complex between the probe and the target sequence. The region of the probes which is used to bind to the target sequence can be made completely complementary to the targeted region of the FCHL1 locus. Therefore, high stringency conditions may be desirable in order to prevent false positives. However, conditions of high stringency are typically used if the probes are complementary to regions of the chromosome which are unique in the genome. The stringency of hybridization is determined by a number of factors during hybridization and during the washing procedure, including temperature, ionic strength, base composition, probe length, and concentration of formamide. Under certain circumstances, the formation of higher order hybrids, such as triplexes, quadraplexes, etc. may be desired to provide the means of detecting target sequences.

Detection, if any, of the resulting hybrid is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinase reaction), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal from the labeled moiety.

Two-step label amplification methodologies are known in the art. These assays work on the principle that a small ligand (such as digoxigenin, biotin, or the like) is attached to a nucleic acid probe capable of specifically binding FCHL1.

In one example, the small ligand attached to the nucleic acid probe is specifically recognized by an antibody-enzyme conjugate. In one embodiment of this example, digoxigenin is attached to the nucleic acid probe. Hybridization is detected by an antibody-alkaline phosphatase conjugate which turns over a chemiluminescent substrate. In a second example, the small ligand is recognized by a second ligand-enzyme conjugate that is capable of specifically complexing to the first ligand. A well known embodiment of this example is the biotin-avidin type of interactions.

Predisposition to lipid disorder and cancer can be ascertained by testing a suitable biological sample of a human for sequence variations of the FCHL1 gene. For example, a person who has inherited a germline FCHL1 mutation would be prone to develop lipid disorder or cancer. This can be determined by testing DNA from any tissue of the person's body. Most simply, blood can be drawn and DNA extracted from the cells of the blood. In addition, prenatal diagnosis can be accomplished by testing fetal cells, placental cells or amniotic cells for mutations of the FCHL1 gene.

The most definitive test for mutations in a candidate locus is to directly compare genomic FCHL1 sequences from lipid disorder or cancer patients with those from a control population. Alternatively, one could sequence messenger RNA after amplification, e.g., by PCR, thereby eliminating the necessity of determining the exon structure of the candidate gene. See for example, U.S. Pat. No. 5,972,614.

Sequence variations from lipid disorder or cancer patients falling outside the coding region of FCHL I can be detected by examining the non-coding regions, such as introns and regulatory sequences near or within the FCHL1 gene. An early indication that mutations in noncoding regions are important may come from Northern blot experiments that reveal messenger RNA molecules of abnormal size or abundance in lipid disorder or cancer patients as compared to control individuals.

Alteration of FCHL1 mRNA expression can be detected by any techniques known in the art (see above). These include Northern blot analysis, PCR amplification, RNase protection, and gene chip analysis. Diminished or increased mRNA expression indicates an alteration of the wild-type FCHL1 gene.

The lipid disorder and cancer condition can also be detected on the basis of the alteration of wild-type FCHL1 polypeptide. For example, the presence of a FCHL1 gene variant which produces a protein having a loss of function, or altered function, may directly correlate to an increased risk of lipid disorder or cancer. Such variation can be determined by sequence analysis in accordance with conventional techniques. For example, antibodies may be used to detect differences in, or the absence of, FCHL1 polypeptides. Antibodies may immunoprecipitate FCHL1 proteins from solution as well as react with FCHL1 protein on Western or immunoblots of polyacrylamide gels. Antibodies may also detect FCHL1 proteins in paraffin or frozen tissue sections, using immunocytochemical techniques.

Functional assays, such as protein binding determinations, can be used. Finding a mutant FCHL1 gene product indicates an alteration of a wild-type FCHL1 gene.

VI. Drug Screening

This invention is also useful for screening compounds by using the HYPLIP1 or FCHL1 polypeptide or binding fragment thereof in any of a variety of drug screening techniques.

The HYPLIP1 or FCHL1 polypeptide employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, for the formation of complexes between a HYPLIP1 or FCHL1 polypeptide and the agent being tested, or examine the degree to which the formation of a complex between a HYPLIP1 or FCHL1 polypeptide and a known ligand is interfered with by the agent being tested.

Thus, the present invention provides methods of screening for drugs comprising contacting such an agent with a HYPLIP1 or FCHL1 polypeptide and assaying (i) for the presence of a complex between the agent and the HYPLIP1 or FCHL1 polypeptide, or (ii) for the presence of a complex between the HYPLIP1 or FCHL1 polypeptide and a ligand, by methods well known in the art. In such competitive binding assays the HYPLIP1 or FCHL1 polypeptide is typically labeled. Free HYPLIP1 or FCHL1 polypeptide is separated from that present in a protein:protein complex, and the amount of free (i.e., uncomplexed) label is a measure of the binding of the agent being tested to FCHL1 or its interference with FCHL1:ligand binding, respectively.

Other suitable techniques for drug screening may provide high throughput screening for compounds having suitable binding affinity to the HYPLIP1 or FCHL1 polypeptides. For example, large numbers of different small peptide test compounds are synthesized on a solid substrate, such as plastic pins or some other surface. The peptide test compounds are reacted with HYPLIP1 or FCHL1 polypeptide and washed. Bound HYPLIP1 or FCHL1 polypeptide is then detected by methods well known in the art.

Purified HYPLIP1 or FCHL1 polypeptide can be coated directly onto plates for use in the aforementioned drug screening techniques. However, non-neutralizing antibodies to the polypeptide can be used to capture antibodies to immobilize the HYPLIP1 or FCHL1 polypeptide on the solid phase.

This invention also contemplates the use of competitive drug screening assays in which neutralizing antibodies capable of specifically binding the HYPLIP1 or FCHL1 polypeptide compete with a test compound for binding to the HYPLIP1 or FCHL1 polypeptide. In this manner, the antibodies can be used to detect the presence of any peptide which shares one or more antigenic determinants of the HYPLIP1 or FCHL1 polypeptide.

A further technique for drug screening involves the use of host eukaryotic cell lines or cells which have a nonfunctional HYPLIP1 or FCHL1 gene. These host cell lines or cells are defective at the HYPLIP1 or FCHL1 polypeptide level. The host cell lines or cells are grown in the presence of drug compound. The rate of growth of the host cells is measured to determine if the compound is capable of regulating the growth of HYPLIP1 or FCHL1 defective cells.

Briefly, a method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Test substances may also be screened for ability to interact with the polypeptide, e.g., in a yeast two-hybrid system. This system may be used as a coarse screen prior to testing a substance for actual ability to modulate activity of the polypeptide. Alternatively, the screen could be used to screen test substances for binding to a HYPLIP1 or FCHL1 specific binding partner, or to find mimetics of a HYPLIP1 or FCHL1 polypeptide.

VII. Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. In one approach, one first determines the three-dimensional structure of a protein of interest (e.g., HYPLIP1 or FCHL1 polypeptide) or, for example, of the FCHL1-receptor or ligand complex, by x-ray crystallography, by computer modeling or most typically, by a combination of approaches. Useful information regarding the structure of a polypeptide may also be gained by modeling based on the structure of homologous proteins. In addition, peptides (e.g., HYPLIP1 or FCHL1 polypeptide) are analyzed by an alanine scan. In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Thus, one may design drugs which have, e.g., improved FCHL1 polypeptide activity or stability or which act as inhibitors, agonists, antagonists, etc. of FCHL1 polypeptide activity. By virtue of the availability of cloned FCHL1 sequences, sufficient amounts of the FCHL1 polypeptide may be made available to perform such analytical studies as x-ray crystallography. In addition, the knowledge of the FCHL1 protein sequence provided herein will guide those employing computer modeling techniques in place of, or in addition to x-ray crystallography.

Following identification of a substance which modulates or affects polypeptide activity, the substance may be investigated further. Furthermore, it may be manufactured and/or used in preparation, i.e., manufacture or formulation, or a composition such as a medicament, pharmaceutical composition or drug. These may be administered to individuals.

Thus, the present invention extends in various aspects not only to a substance identified using a nucleic acid molecule as a modulator of polypeptide activity, in accordance with what is disclosed herein, but also a pharmaceutical composition, medicament, drug or other composition comprising such a substance, a method comprising administration of such a composition comprising such a substance, a method comprising administration of such a composition to a patient, e.g., for treatment of lipid disorder or cancer, use of such a substance in the manufacture of a composition for administration, e.g., for treatment of lipid disorder or cancer, and a method of making a pharmaceutical composition comprising admixing such a substance with a pharmaceutically acceptable excipient, vehicle or carrier, and optionally other ingredients.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g., by substituting each residue in turn. Alanine scans of peptide are commonly used to refine such peptide motifs. These parts or residues constituting the active region of the compound are known as its pharmacophore.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially used where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic(s) found by this approach can then be screened to see whether they have the target property, or to what extent they exhibit it. Further optimization or modification can then be carried out to arrive at one or more final mimetics for in vivo or clinical testing.

VIII. Gene Therapy

According to the present invention, a method is also provided of supplying wild-type FCHL1 function to a cell which carries mutant FCHL1 alleles. The wild-type FCHL1 gene or a part of the gene may be introduced into the cell in a vector such that the gene remains extrachromosomal. In such a situation, the gene will be expressed by the cell from the extrachromosomal location. More preferred is the situation where the wild-type FCHL1 gene or a part thereof is introduced into the mutant cell in such a way that it recombines with the endogenous mutant FCHL1 gene present in the cell. Such recombination requires a double recombination event which results in the correction of the FCHL1 gene mutation. Vectors for introduction of genes both for recombination and for extrachromosomal maintenance are known in the art, and any suitable vector may be used. Methods for introducing DNA into cells such as electroporation, calcium phosphate coprecipitation and viral transduction are known in the art, and the choice of method is within the competence of skilled practitioners.

As generally discussed above, the FCHL1 gene or fragment, where applicable, may be employed in gene therapy methods in order to increase the amount of the expression products of such genes in lipid disorder or cancerous cells. Such gene therapy is particularly appropriate, in which the level of FCHL1 polypeptide is absent or compared to normal cells. It may also be useful to increase the level of expression of a given FCHL1 gene even in those situations in which the mutant gene is expressed at a “normal” level, but the gene product is not fully functional.

Gene therapy would be carried out according to generally accepted methods, for example, as described by Cooper, Gene Therapy, BIOS Scientific Publishers, Oxford (1998). Cells from a patient would be first analyzed by the diagnostic methods described above, to ascertain the production of FCHL1 polypeptide in these cells. A virus or plasmid vector, containing a copy of the FCHL1 gene linked to expression control elements and capable of replicating inside the sample cells, is prepared. Suitable vectors are known in the art. The vector is then injected into the patient.

Gene transfer systems known in the art may be useful in the practice of the gene therapy methods of the present invention. These include viral and nonviral transfer methods. A number of viruses have been used as gene transfer vectors, including papovaviruses, e.g., SV40, adenovirus, vaccinia virus, adeno-associated virus, herpes viruses including HSV and EBV; lentiviruses, Sindbis and Semliki Forest virus, and retroviruses of avian, murine, and human origin. Most human gene therapy protocols have been based on disabled murine retroviruses.

Nonviral gene transfer methods known in the art include chemical techniques such as calcium phosphate coprecipitation; mechanical techniques, for example microinjection; membrane fusion-mediated transfer via liposomes; and direct DNA uptake and receptor-mediated DNA transfer. Viral-mediated gene transfer can be combined with direct in vivo gene transfer using liposome delivery, allowing one to direct the viral vectors to the affected cells and not into the surrounding nondividing cells. Alternatively, the retroviral vector producer cell line can be injected into affected cells. Injection of producer cells would then provide a continuous source of vector particles.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

Liposome/DNA complexes have been shown to be capable of mediating direct in vivo gene transfer. While in standard liposome preparations the gene transfer process is nonspecific, localized in vivo uptake and expression may be accomplished following direct in situ administration.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes FCHL1, expression will produce FCHL1. If the polynucleotide encodes an antisense polynucleotide or a ribozyme, expression will produce the antisense polynucleotide or ribozyme. Thus in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Receptor-mediated gene transfer, for example, may be accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. One appropriate receptor/ligand pair may include the estrogen receptor and its ligand, estrogen (and estrogen analogues). These ligand-DNA conjugates can be injected directly into the blood if desired and are directed to the target tissue where receptor binding and internalization of the DNA-protein complex occurs. To overcome the problem of intracellular destruction of DNA, coinfection with adenovirus can be included to disrupt endosome function.

IX. Peptide Therapy

Peptides which have FCHL1 activity can be supplied to cells which carry mutant or missing FCHL1 alleles. Protein can be produced by expression of the cDNA sequence in bacteria, for example, using known expression vectors. Alternatively, FCHL1 polypeptide can be extracted from FCHL1-producing mammalian cells. In addition, the techniques of synthetic chemistry can be employed to synthesize FCHL1 protein. Any of such techniques can provide the preparation of the present invention which comprises the FCHL1 protein. Preparation is substantially free of other human proteins. This is most readily accomplished by synthesis in a microorganism or in vitro.

Active FCHL1 molecules can be introduced into cells by microinjection or by use of liposomes, for example. Alternatively, some active molecules may be taken up by cells, actively or by diffusion. Extracellular application of the FCHL1 gene product may be sufficient. Molecules with FCHL1 activity (for example, peptides, drugs or organic compounds) may also be used to effect such a reversal. Modified polypeptides having substantially similar function are also used for peptide therapy.

X. Transformed or Transfected Hosts

Similarly, cells and animals which carry a mutant HYPLIP1 or FCHL1 allele can be used as model systems to study and test for substances which have potential as therapeutic agents. These may be isolated from individuals with FCHL1 mutations, either somatic or germline. Alternatively, the cell line can be engineered to carry the mutation in the FCHL1 allele.

Animals for testing therapeutic agents can be selected after mutagenesis of whole animals or after treatment of germline cells or zygotes. Such treatments include insertion of mutant HYPLIP1 or FCHL1 alleles, usually from a second animal species, as well as insertion of disrupted homologous genes. Alternatively, the endogenous HYPLIP1 or FCHL1 gene of the animals may be disrupted by insertion or deletion mutation or other genetic alterations using conventional techniques to produce knockout or transplacement animals. A transplacement is similar to a knockout because the endogenous gene is replaced, but in the case of a transplacement the replacement is by another version of the same gene. After test substances have been administered to the animals, the phenotype must be assessed. If the test substance prevents or suppresses the disease, then the test substance is a candidate therapeutic agent for the treatment of disease. These animal models provide an important testing vehicle for potential therapeutic products.

In one embodiment of the invention, transgenic animals are produced which contain a functional transgene encoding a functional HYPLIP1 or FCHL1 polypeptide or variants thereof. Transgenic animals expressing HYPLIP1 or FCHL1 transgenes, recombinant cell lines derived from such animals and transgenic embryos may be useful in methods for screening for and identifying agents that induce or repress function of FCHL1. Transgenic animals of the present invention also can be used as models for studying indications such as lipid disorder.

In one embodiment of the invention, a HYPLIP1 or FCHL1 transgene is introduced into a non-human host to produce a transgenic animal expressing a human or murine FCHL1/HYPLIP1 gene. The transgenic animal is produced by the integration of the transgene into the genome in a manner that permits the expression of the transgene. Methods for producing transgenic animals are generally described in “Manipulating the Mouse Embryo; A Laboratory Manual” 2nd edition (eds., Hogan, Beddington, Costantimi and Long, Cold Spring Harbor Laboratory Press, 1994).

It may be desirable to replace the endogenous FCHL1 by homologous recombination between the transgene and the endogenous gene; or the endogenous gene may be eliminated by deletion as in the preparation of “knock-out” animals. Typically, a FCHL1 gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which express a mutant form of the polypeptide. Techniques of gene targeting and preparing transgenic mouse are described in Joyner, Gene Targeting. A Practical Approach, 2^{nd} Ed., Oxford University Press (2000).

As noted above, transgenic animals and cell lines derived from such animals may find use in certain testing experiments. In this regard, transgenic animals and cell lines capable of expressing wild-type or mutant FCHL1 may be exposed to test substances. These test substances can be screened for the ability to reduce overepression of wild-type FCHL1 or impair the expression or function of mutant FCHL1.

XI. Pharmaceutical Compositions and Routes of Administration

The FCHL1 polypeptides, antibodies, peptides and nucleic acids of the present invention can be formulated in pharmaceutical compositions, which are prepared according to conventional pharmaceutical compounding techniques. See, for example, Remington's Pharmaceutic Sciences, 18th Ed. (Mack Publishing Co., Easton, Pa. (1990)). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be nontoxic and should not interfere with the efficacy of the active ingredient. The carrier may take a wide variety of forms depending on the form of preparation desired for administration, e.g., intravenous, oral, intrathecal, epineural or parenteral.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

Instead of administering these agents directly, they could be produced in the target cell, e.g. in a viral vector such as described above or in a cell based delivery system designed for implantation in a patient. The vector could be targeted to the specific cells to be treated, or it could contain regulatory elements which are more tissue specific to the target cells. The cell based delivery system is designed to be implanted in a patient's body at the desired target site and contains a coding sequence for the active agent. Alternatively, the agent could be administered in a precursor form for conversion to the active form by an activating agent produced in, or targeted to, the cells to be treated.

### EXAMPLES

The following examples further illustrate the present invention. These examples are intended merely to be illustrative of the present invention and are not to be construed as being limiting.

### Example 1

Mice and diets. The development of the recombinant congenic (RC) mouse strains was described in Demant, et al., Immuunogenetics 24:416-422 (1986). Each RC strain contains a distinct part (approximately 12.5%) of the donor strain (C57BL/10ScSnA) genome and approximately 87.5% of the background strain (C3H/DiSnA). HcB-19 animals were unavailable for breeding. Thus, (HcB-19×BALB/c)F1 mice were used for breeding to the CAST/Ei mice. Progeny were genotyped for polymorphic markers D3Mit29, D3Mit76, D3Mit75, and D3Mit121 to exclude animals with BALB/c alleles within or near the HYPLIP1 region. Animals with HcB-19 alleles were intercrossed to produce progeny which are essentially (HcB-19×CAST/Ei)F2s at the HYPLIP1 locus. These animals are referred to as “(HcB-19×CAST/Ei)F2” mice. All mice were housed in groups of five or less animals per cage and maintained on a 12 hour light-dark cycle at an ambient temperature of 23° C. They were allowed ad libitum access to water and standard Purina Rodent Chow (Ralston-Purina Co.) containing 4% fat as described in Hedrick, et al., J. Biol. Chem. 269:20676-20682 (1993).

Plasma lipids insulin and lipases. Mice were fasted for 12 h prior to retro-orbital bleeding, and were at bled 3-6 h after the beginning of the light cycle under isofluorance anesthesia using EDTA as the anticoagulant. Plasma lipids were determined as described in Hedrick et al., supra (1993). Plasma lipoproteins were fractionated from 400 μl samples of whole pooled plasma by gel filtration chromatography using a Pharmacia FPLC system (Pharmacia LKB Biotechnology) with two Superose 6 columns connected in series. Fractions of 0.5 ml were collected and the cholesterol and triglyceride content of each fraction determined. Plasma insulin levels were determined in HcB-19 mice and mice from both parental strains in duplicate measurements using an insulin RIA kit (Linco Research, Inc.). Lipoprotein lipase and hepatic lipase activities were determined in post-heparin plasma after administration of heparin via tail-vein injection (Doolittle et al., J. Lipid Res. 281326-1333 (1993)). Levels of ketone body β-hydroxybutyrate were determined using a kit (Sigma) according to the manufacturer's instructions, except all reagent volumes were scaled down accordingly to measure levels in the small sample volumes of mouse plasma. Blood collection tubes were pre-chilled on ice and the samples centrifuged within 5 minutes to remove erythrocytes in order to obtain accurate plasma lactate concentrations, which were measured in duplicate using a kit (#735-10, Sigma). For pyruvate determinations, EDTA was not used since whole blood was immediately deproteinized after bleeding and the pyruvate measured using a kit (#726-UV, Sigma) according to the manufacturer's instructions. Blood collection tubes were pre-chilled on ice with cold perchloric acid and the blood-precipitate mixture was kept cold for at least 5 minutes to ensure complete protein precipitation for pyruvate measurements.

Rates of VLDL secretion. Plasma triglyceride concentrations were determined both before, and 30 and 60 min after administering Triton WR-1339 (Sigma) by tail-vein injection to mice which had been fasted overnight. Khan et al., Biochem. Biophys. Acta 1044:297-304 (1990). The net difference in plasma triglyceride levels before and after administration of the Triton WR-1339 represents the amount of triglyceride secreted during that time interval.

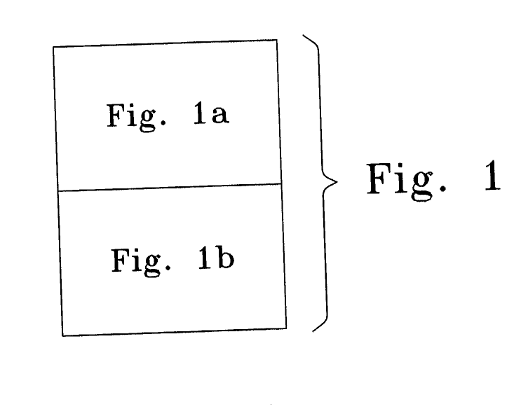

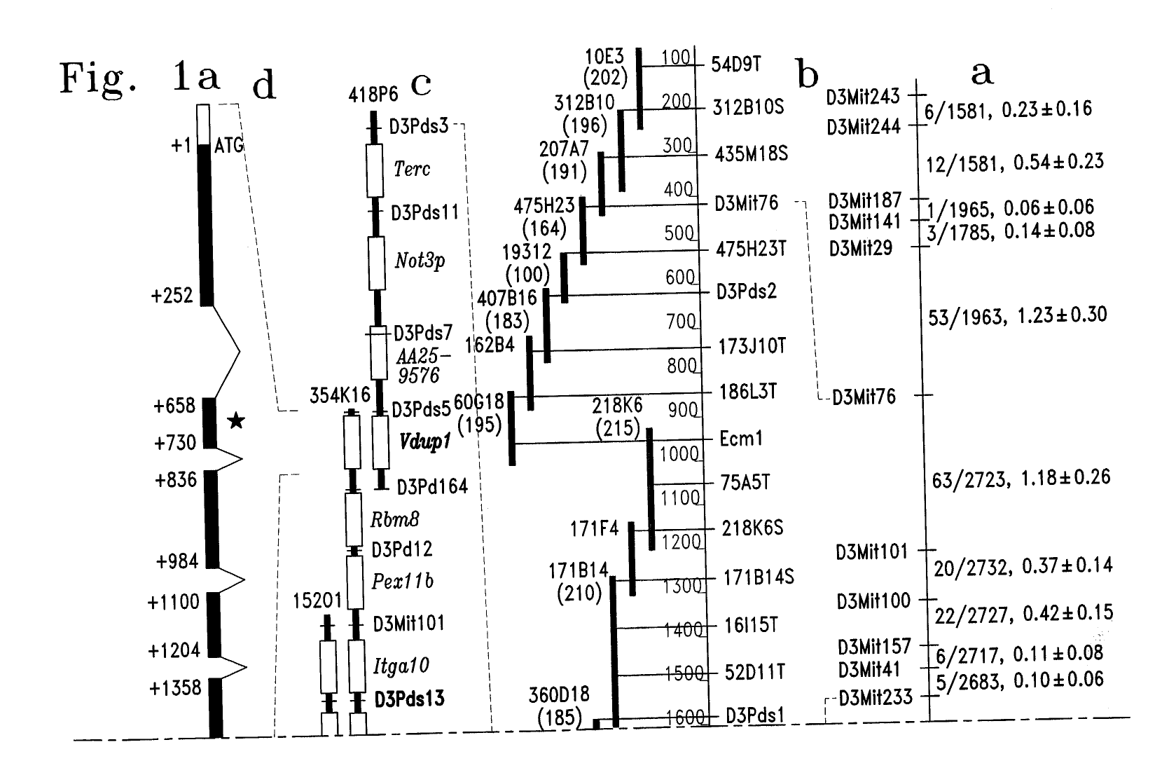

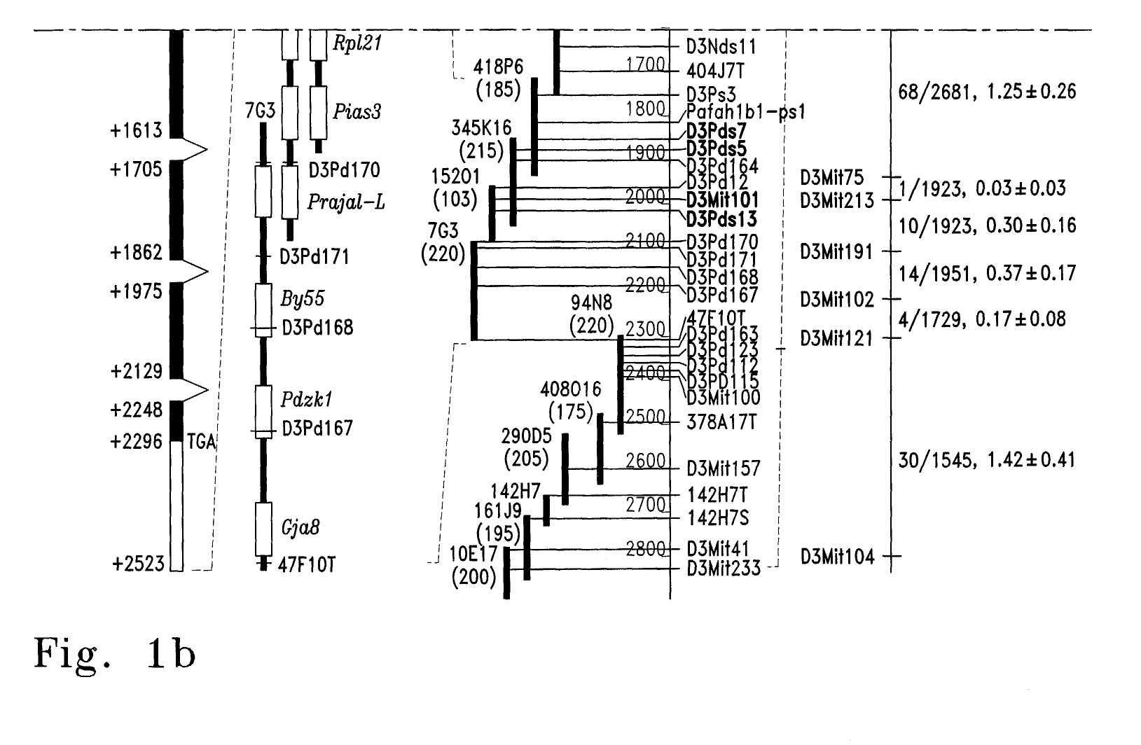

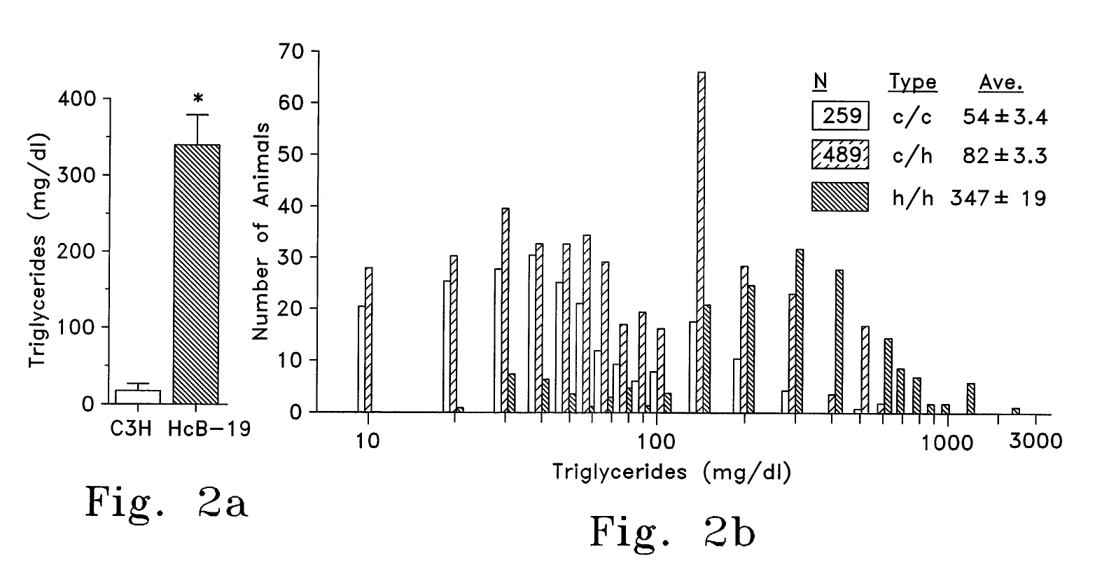

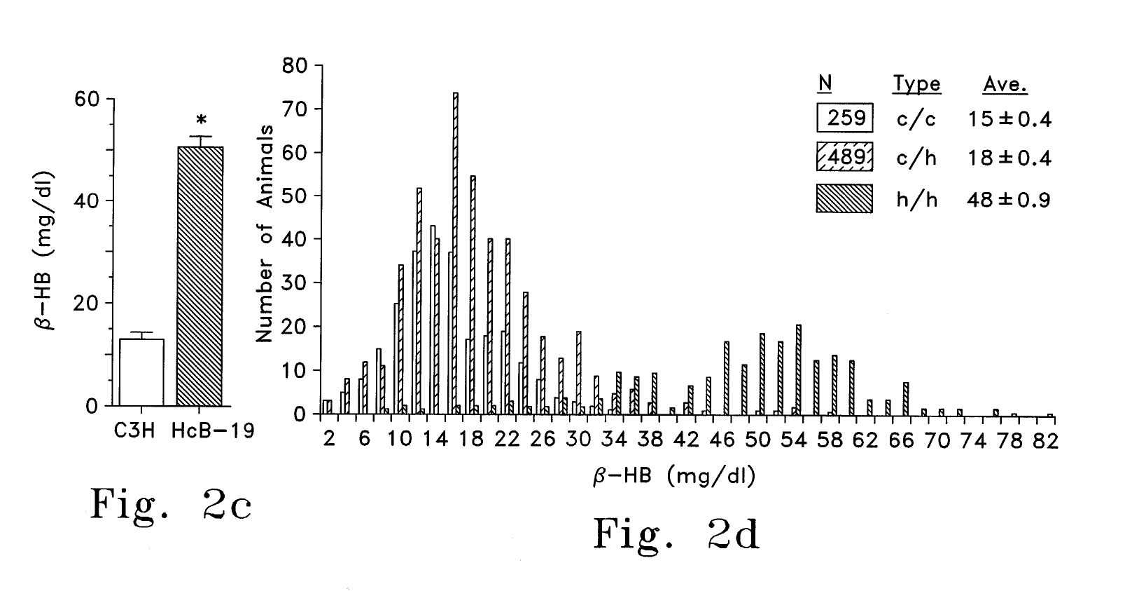

Fine mapping of HYPLIP1. In order to fine map the HYPLIP1 locus, a large F2 intercross was constructed between the mutant strain HcB-19 and the evolutionarily distant strain CAST/Ei, since most known microsatellite markers in the HYPLIP1 region are polymorphic between these two strains. Over two thousand (HcB-19×CAST/Ei)F2 mice were generated and genotyped for HYPLIP1 microsatellite markers (D3Mit29, D3Mit76, D3Mit 101, D3Mit100, D3Mit157, D3Mit233, D3Mit41, and D3Mit75). These markers were radiation hybrid mapped to establish their exact order and intermarker distances (FIG. 1). Triglycerides levels, which yielded the highest lod score in our previously reported (HcB-19×C57BL/10ScSnA)F2 cross, were measured for approximately half of the (HcB-19×CAST/Ei)F2 animals (FIG. 2). As evident, there is considerable overlap in triglyceride levels between the three genotypic groups, making the assignment of recombinant animals to a particular HYPLIP1 genotypic class difficult to assess based solely upon their plasma triglyceride value. Therefore, additional phenotypes were searched to analyze in this cross.

In the previous cross of 183 (HcB-19×C57BL/10ScSnA)F2 animals, the HYPLIP1 locus was linked to plasma triglycerides, VLDL+LDL cholesterol, unesterified cholesterol, total cholesterol, and free fatty acid (FFA) levels with lod scores of 30.5, 22.4, 21.3, 10.2, and 9.2; respectively, at peak marker D3Mit101 (Castellani et al., 1998, supra and data not shown). Since plasma FFA levels are elevated approximately 60% in HcB-19 over the parental C3H strain, and since fatty acids are subsequently either esterified to produce triglycerides or oxidized to form ketone bodies, we examined the predominant ketone body, β-hydroxybutyrate (β-HB), in HcB-19 and its C3H parental control (FIG. 2). Plasma levels of ketone body β-HB were elevated approximately four-fold in HcB-19 animals over the C3H parental strain (FIG. 2). We therefore measured β-hydroxybutyrate levels in mice from the (HcB-19×CAST/Ei)F2 cross, and found that ketone body levels segregated with the HYPLIP1 locus, yielding a peak lod score of 227 at marker D3Mit101, while triglyceride levels yielded a peak lod score of 91 for this same marker. Importantly, the variability and overlap in ketone body levels between the three genotypic classes is much less than that for triglycerides, making the analysis of the HYPLIP1 genotype of recombinant animals more certain. Therefore, both ketone body and triglyceride levels were examined in animals with crossovers between markers D3Mit76 and D3Mit75 in order to restrict the location of the HYPLIP1 gene.

Northern blot analysis and RT-PCR. Total RNA was isolated from liver with Trizol reagent from Life Technologies according to the manufacturer's instructions. PolyA RNA was isolated using the Oligotex mRNA kit from Qiagen. PolyA RNA (2 ug) was resolved by electrophoresis in a denaturing agarose gel using the NorthernMax protocol and reagents from Ambion and transferred to Brightstar-Plus membranes (Ambion) according to the manufacturer's instructions. DNA was labeled with ³²P-dCTP from Amersham Pharmacia Biotech using the random primer kit from Life Technologies. Filters were hybridized and washed according to the NorthernMax protocol. The filters were exposed overnight and analyzed using the Storm Image Analysis System from Molecular Dynamics. RT-PCR was done on total RNA using the Access RT-PCR kit from Promega.

Statistical Analysis. Since the triglyceride and ketone body levels of heterozygous mice overlap with both wild type and the HYPLIP1 mutant homozygous groups, recombinant mice were evaluated statistically using logistic regression. Predictive probabilities were calculated using the logistic subroutine of the SAS program (Version 6.10, 1993, SAS Institute Inc.). Given the distribution of triglyceride and ketone body levels for each genotype from animals that were non-recombinant between markers D3Mit76 and D3Mit100, logistic regression coefficients were calculated by using the ketone body values and the natural log of the triglyceride values. (The natural log was used to normalize the data set.) These logistic regression coefficients were then used to calculate the probability that each parental recombinant animal was heterozygous and each recombinant backcross progeny was homozygous for HYPLIP1, given the sex-adjusted triglyceride and ketone body values. The predicted probabilities thus represent an estimate of the probability that a particular mouse is heterozygous [P(c/h)] or homozygous [P(h/h)] for the HYPLIP1 gene given its phenotype. Linkage data were analyzed and lod scores and recombination distances were calculated by using the Map Manager QT v.3.0 Program.

Radiation Hybrid Mapping Genotyping, and Primers. Radiation hybrid mapping was performed using the mouse/hamster T31 radiation hybrid panel (Research Genetics, Inc.). All clone lines producing a breakpoint were typed in duplicate, as were any ambiguous typings. PCR and thermal cycling conditions were as recommended by the manufacturer. All mapping data are available at The Jackson Laboratory Mouse Radiation Hybrid database (http://www.jax.org/resources/documents/cmdata/rhmap/). Automated genotyping of DNA microsatellite markers was performed using fluorescent-labeled primers (Research Genetics) and ABI 377 machines according to standard protocols.

BAC Contig Construction. BACs for the HYPLIP1 region were identified by hybridization of labeled PCR products from the critical region to the RPCI-23 mouse BAC library (Children's Hospital, Oakland). Briefly, high-density filters with a 1×coverage were hybridized with random-primed ³²P-labeled probes (1×10⁶ cpm/ml hybridization solution). Ten to twenty PCR products were routinely pooled per hybridization. Filters were pre-hybridized for one hour and hybridized overnight (16-18 hours) at 65° C. Filters were washed at 65° C. for 4 to 6 times until essentially all non-bound probe was removed. The filters were then exposed to phosphor screens (Molecular Dynamics) for 2 to 24 hrs and analyzed on the Storm Image Analysis System (Molecular Dynamics). The positions of the positive clones were interpreted according to the manufacturer's instructions using the transparent overlays as an orientation guide. The order of the markers was based on RH mapping data and their presence or absence within each BAC clone bin. BAC ends were sequenced for primer design, PCR amplified, and then subsequently used for chromosome walking and gap closure of the ˜3 Mb contig constructed between markers D3Mit76 and D3Mit157.

Sequencing and Sequence Data Analysis. BAC DNA was extracted using a standard cesium chloride cushion according to Sambrook et al., Molecular Cloning, A Laboratory Manual, 3^{rd} Edition (2000). For sequencing, a sub-library in pUC18 was first constructed from each BAC. Briefly, BAC DNA was randomly sheared using a sonicator and end filled with Klenow, then size fractionated by agarose gel electrophoresis, and fragments between 1.5-3.0 kb were collected. The gel purified fragements were cloned into SmaI-cut, bacterial alkaline phosphatase treated pUC 18. Ligation (Roche Rapid Ligation kit) and transformation of XL-10 competent cells (Stratagene) were done according to the manufacturer's instructions. Several thousand clones were picked from each BAC for sequencing. Plasmid DNA was extracted using the Qiagen Biorobot. Cycle sequencing was performed using BigDye terminators (DNA sequencing kit, PE Applied Biosystems), purified on Centrisep spin columns in a 96-well format and analysed on an ABI 3700 calillary sequencer (PE Applied Biosystems). PCR products were purified using a PCR purification kit (Qiagen) and 30-60 ng was used for cycle sequencing. Raw sequence was analyzed and assembled using Phred and Phrap, vector and repeat sequences were masked and high quality sequence was used for BLAST against internal and external databases.

Metabolism of ¹⁴C-oleate in Liver Slices. Following an overnight fast, mice were anesthetized with pentobarbital (50 mg/kg) and the liver removed. A Staddie-Riggs microtome was used to obtain fresh liver slices (˜0. 5 mm thick) which were immediately weighed and incubated for 1 hour under 95% O₂:5% CO₂ in Krebs-Henseleit buffer containing 5.5 mM glucose and a 3% BSA/1 mM ¹⁴C-oleic acid complex (Olubadewo, et al., Biochem Pharmacol. 45:2441-2447 (1993)). The final specific activity was 250,000 dpm/μmol. ¹⁴CO₂ production was determined by using hyamine hydroxide to trap the CO₂ and measuring the radioactive counts in a liquid scintillation spectrometer essentially as described (Olubadewo, et al., 1993, supra)). The ¹⁴C-oleic acid incorporation into ketone bodies and secreted triglycerides was determined in the liver slice incubations under similar conditions except the cells were continuously gassed with 95% 02:5% CO₂ and no hyamine hydroxide was used. After the 40 min incubation, the media was removed for extraction of lipids and ketone bodies. Radioactivity incorporated into ketone bodies was measured following perchloric acid deproteinization as previously described (Olubadewo, et al., 1993, supra)). Triglycerides were separated from the media lipid extracts by thin layer chromatography, and the radioactivity in the band corresponding to triglycerides was determined as described (Castellani, et al., Biochim Biophys Acta. 1086:197-208 (1991)).

Hepatocytes Isolation and Measurement of Secreted Apolipoprotein B HcB-19 and C3H hepatocytes were isolated by recirculating perfusion of livers (Doolittle, et al., J. Lipid Res. 28:1326-1333 (1987)). Hepatocytes were cultured at 37° C. under 5% CO₂ in Williams Media E (Gibco BRL)/5% FBS (Sigma)/10 mM HEPES, pH 7.4 (Calbiochem)/0.2 mg/ml gentamycin sulfate (Sigma) overnight, then changed to serum free media (Lanford, et al., Methods in Molecular Medicine: Hepatitis C Protocols. Totowa, N.J.: Humana Press, Inc. Ed. Lau, J. Y. N. pp. 501-515 (1998)). Primary hepatocytes were incubated for 3 h in the presence of ³⁵S-methionine. Cells and conditioned media samples were collected and apoB isolated by immunoprecipitation and SDS-PAGE. The amount of apoB was determined by exposing the dried gel to a phosphorimager screen and the apoB bands quantified by using ImageQuant software. The results for each condition were normalized to total cellular TCA counts.

### EXAMPLE 2

Ketogenesis

Triglycerides, ketone bodies and free fatty acids (FFA) were elevated in the HYPLIP1 mutant mice. Increased plasma FFA can result in increased flux through all FFA metabolic pathways, causing increased esterification into triglycerides. This may cause hyperlipidaemia and increased VLDL, as well as increased beta-oxidation, causing increased ketogenesis and elevated ketone bodies. Increased plasma FFA can be caused by increased lipolysis, or due to the hypertriglyceridemia in the HcB-19 mice. Ketone levels in these mice were measured for two reasons: 1) To assay for mitochondrial HMG-CoA synthase (Hmgcs2) which was mapped to the 2.7 cM HYPLIP1 locus and 2) plasma FFA levels were increased in (HcB-19×B10) F2 animals homozygous for HcB-19 (HYPLIP1) alleles at the HYPLIP1 locus. In order to measure ketone bodies in HcB-19 animals, 3-hydroxybutyrate (also known as beta-hydroxybutyrate) was assayed using beta-hydroxybutyrate dehydrogenase to catalyze the oxidation of betahydroxybutyrate to acetoacetate. During this oxidation, an equimolar amount of nicotinamide adenine dinucleotide (NAD) is reduced to NADH, which absorbs light at 340 normalization. Thus, an increase in absorbance at 340 normalization is directly proportional to the betahydroxybutyrate concentration in the sample. The ketone and triglyceride levels measured in selected recombinant animals in shown in Table 1.

### EXAMPLE 3

Statistical Analysis of Recombinant Animals

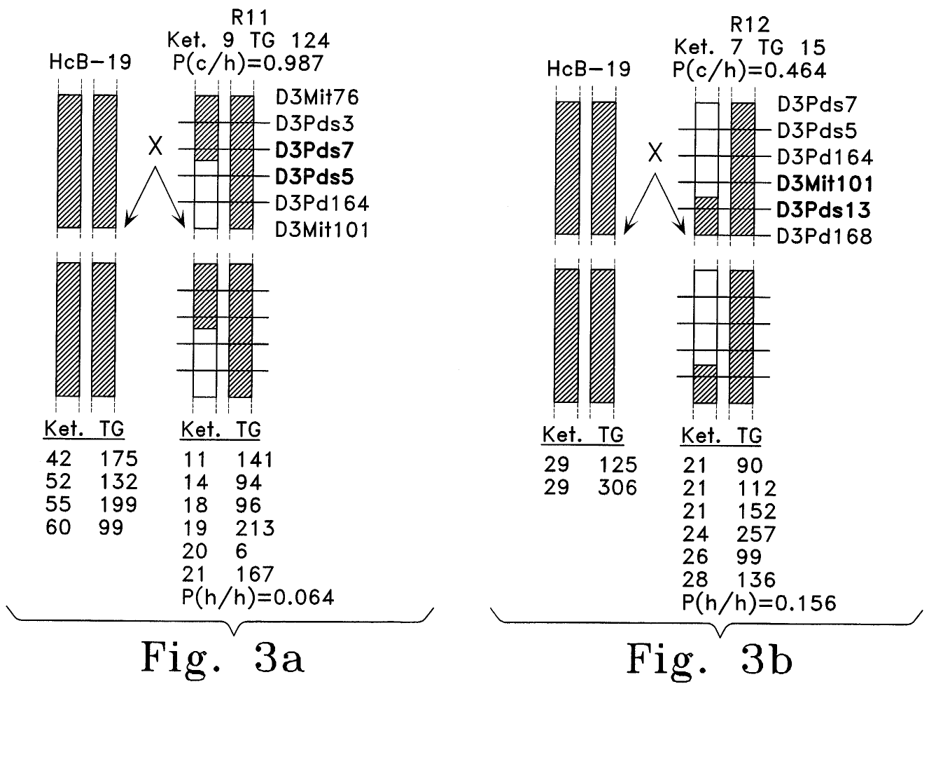

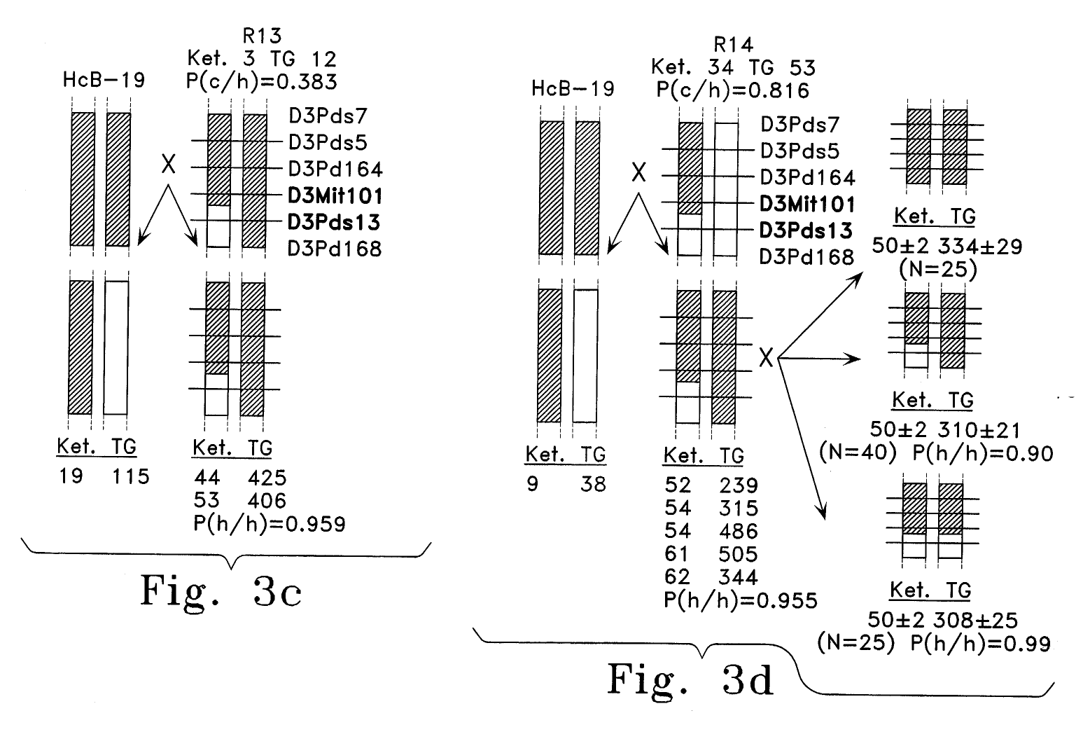

Around 230 animals with recombinations between markers D3Mit76 and D3Mit75 were generated from approximately two thousand (HcB-19×CAST/Ei)F2s. Since subsequent recombinant analysis results restricted the location of HYPLIP1 between microsatellite markers D3Mit76 and D3Mit100 (data not shown), only recombinant animals with crossovers between these markers were backcrossed to HcB-19 for progeny testing. Progeny testing by backcross to the mutant HcB-19 strain was conducted in order to confirm the HYPLIP1 genotype of each recombinant animal. This was done by analysis of ketone body and triglyceride levels of all backcross progeny which inherited the chromosome with the same crossover as the original recombinant parent. The likelihood that a particular crossover type had a copy of the HYPLIP1 mutation was assessed by using logistic regression analysis. The probability of each recombinant animal and their backcross progeny being homozygous for the HYPLIP1 gene was calculated by determining predictive probabilities (FIG. 3). The current genetic region for the HYPLIP1 gene has been narrowed to a 115 kb region between markers AA25957 and Pds13.

Marker Development: The approximate distance between the peak marker D3Mit101 and the nearest proximal marker, D3Mit76, is about 1200 kb, and between marker D3Mit101 and the nearest distal marker, D3Mit100, is around 240 kb. Since these distances are large, particularly between D3Mit76 and D3Mit101, it was necessary to identify new polymorphic markers between HcB-19 and CAST/Ei in order to fine map the locations of crossovers. Thus, single nucleotide variants (SNVs) between HcB-19 and CAST/Ei were identified by genomic sequencing using primers designed from BAC sequence obtained from the physical mapping. The map positions of SNVs identified between HcB-19 and CAST/Ei are shown in FIG. 1.

Analysis of recombinant animals and their HcB-19 backcross progeny support a localization of the HYPLIP1 gene between proximal SNV marker AA25957 and distal SNV marker Pd167, a distance of approximately 115 kb.

### EXAMPLE 4

Sequencing

Four BACs within the critical region were sequenced with 6× coverage. As shown in FIG. 1, BLAST analysis of BACs 418P6, 354K16, 15201, and 7G3 from the HYPLIP1 critical region revealed thirteen known genes: Terc, KIAA, AA259576, Vdup1, Rbm8, Pex11, Int10, Rp121, Pias3, Praja1L, By55, Pdzk1, and Muscx. Of these, KIAA, Vdup1, Rbm8 and Pex11 fall inside the HYPLIP1 critical interval. In addition, three expressed sequence tag (EST) sequences were also identified from BAC 354K16, which contains the peak lod score marker for ketone body and triglyceride levels, D3Mit101. Candidate genes were evaluated by Northern analysis and/or sequencing of RT-PCR products to identify possible mRNA expression or sequence differences between the mutant strain HcB-19 and its normolipidemic parental control C3H. Rbm8 and Int10 were eliminated from the candidate gene list on the basis of their known functions.

### EXAMPLE 5

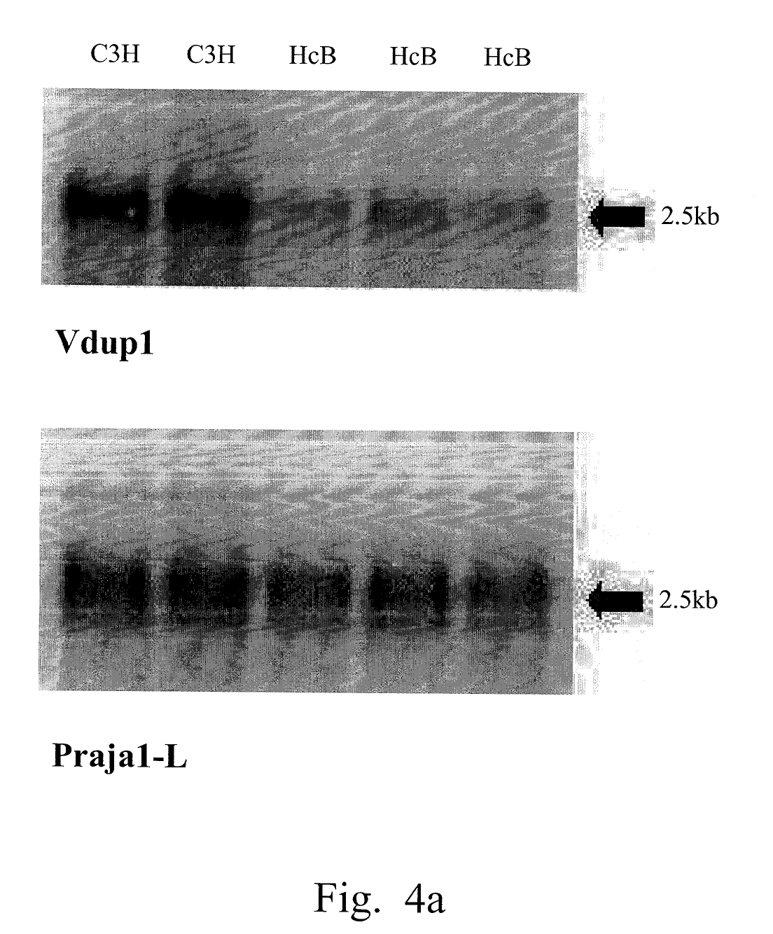

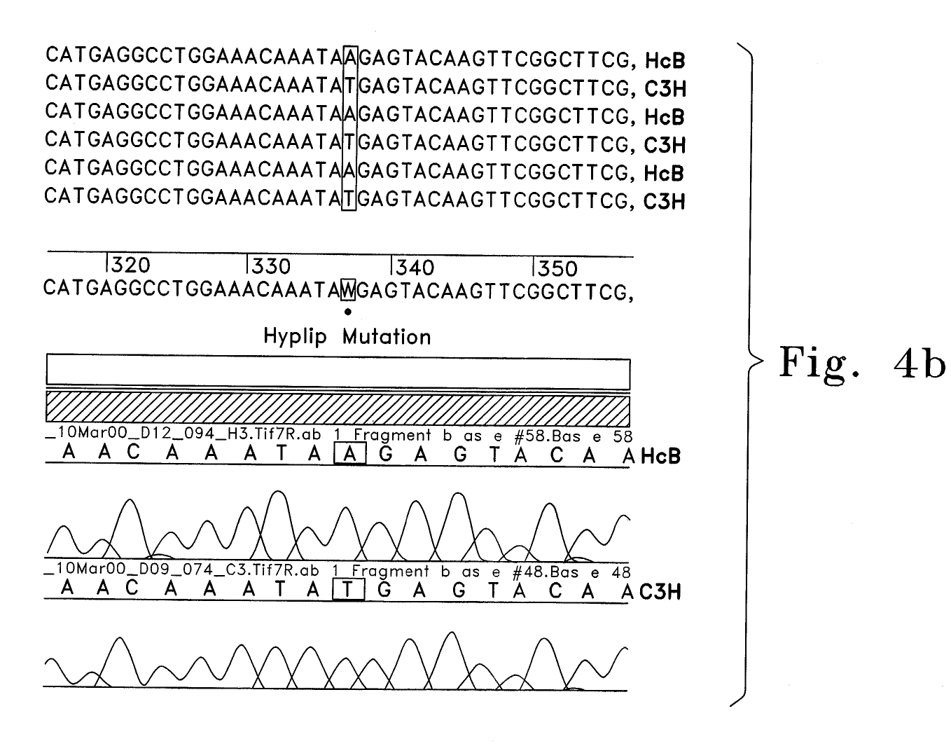

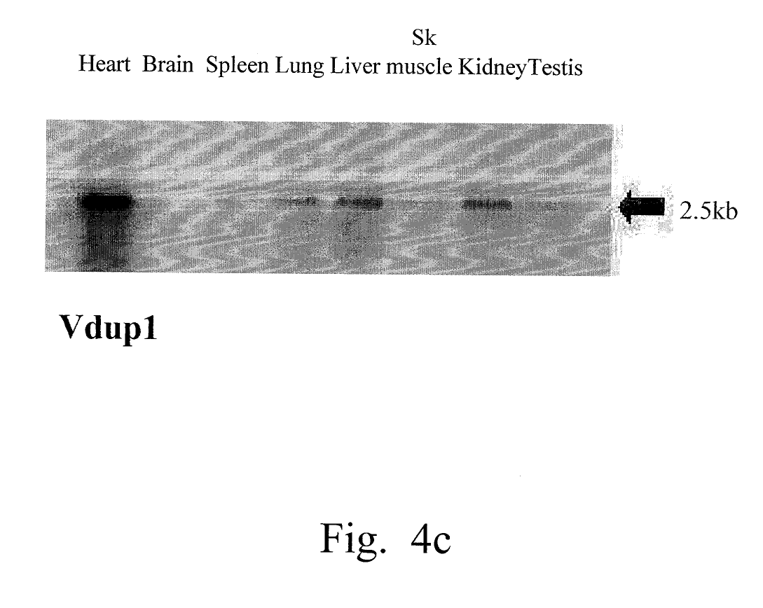

Mutation Detection Probes made from several candidate genes (Pex11, Pias3, W35051 and Vdup1) were scrutinized using polyA Northerns for their expression profile. The expression of Vdup1 was found to be reduced in the liver of three affected animals compared to normal age matched control animals (FIG. 4). Primers designed from several cDNAs (Pias3, Pex11, Vdup1) were tested by RT-PCR with liver RNA from normal and affected animals, followed by sequencing. A point mutation in the Vdup1 transcript was detected in all three affected animals and not in the controls (FIG. 4). The polymorphism altered a tyrosine residue (TAT) at position 97 (of 395 aa) to a stop codon (TAA). Reduced expression of this gene in the affected animals may be due to mRNA surveillance, a mechanism that degrades aberrant mRNAs in eukaryotic cells. Primers across the mutation that amplifies genomic DNA were designed and several F2 animals were found to exhibit the mutation in the homozygous or heterozygous state. In addition, 96 inbred strains of mice were also sequenced for this region and were found to have no polymorphisms for this nucleotide. Furthermore, from comparison of the sequencing results of over 200 kb of fully aligned, contiguous genomic sequence with two-fold coverage from HcB-19 and C3H cosmid libraries, the only sequence difference observed was the Vdup1 nonsense mutation.

Vdup1 is composed of eight exons spanning approximately 5 kb (FIG. 1d). The HcB-19 nonsense mutation occurs in exon two, at codon 97 (FIG. 1d). The Vdup1 transcript was fairly ubiquitously expressed in all tissues examined, with the highest abundance in heart, liver, and kidney (FIG. 4c). The decrease in Vdup1 mRNA in HcB-19 may result from nonsense-mediated mRNA decay through RNA surveillance mechanisms for the detection and degradation of transcripts with premature stop codons (Culbertson, Trends Genet. 15:74-80 (1999) and Leeds et al. Genes Dev. 5:2303-2314 (1991)).

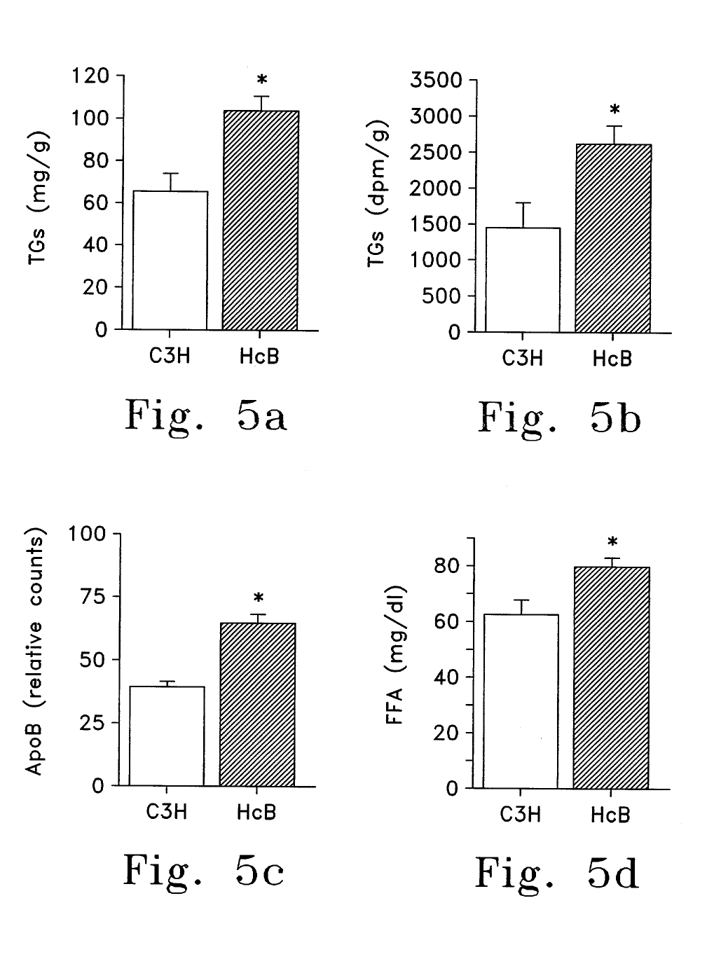

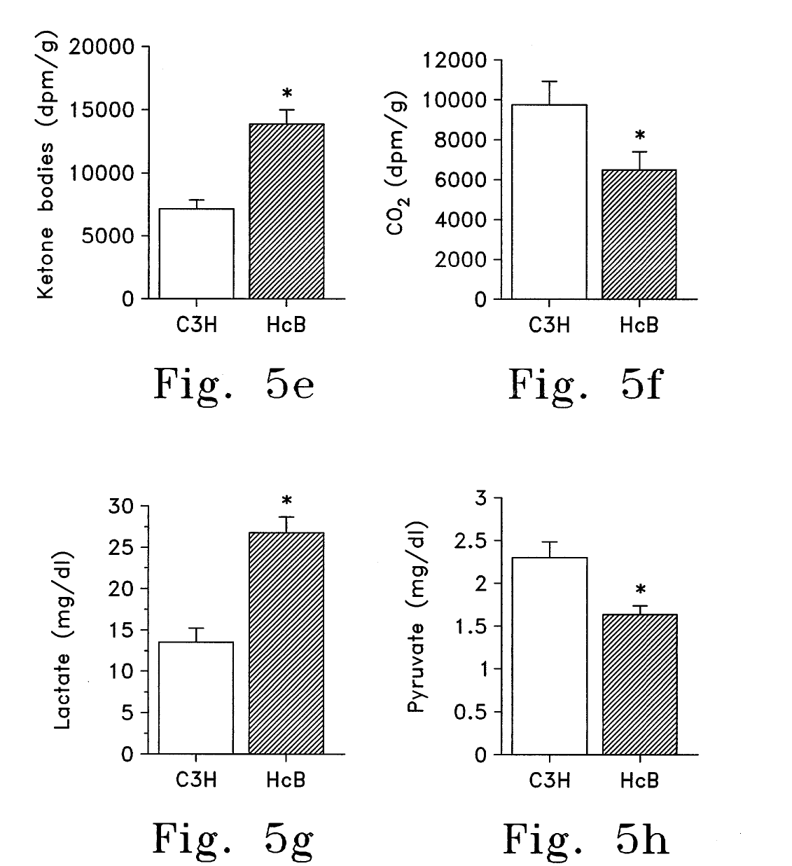

The nonsense mutation in Vdup1 affects several aspects of lipid metabolism. In HYPLIP1 mutant mice, triglyceride secretion in vivo is increased ˜70%, consonant with the elevation in plasma triglycerides (Castellani et al., 1998, supra.) Consistent with this, an ˜70% increase in total triglyceride content of HcB-19 livers was found (FIG. 5a). In addition, from liver slice experiments, the incorporation of ¹⁴C-oleate into newly-synthesized triglycerides was increased 70% in HcB-19 (FIG. 5b). Furthermore, secretion of apoB in hepatocyte cultures isolated from perfused livers was also elevated 70% in HcB-19 (FIG. 5c).

HYPLIP1 mice have elevated plasma FFA levels (FIG. 5d), which would be expected to increase the supply of exogenous fatty acids to the liver since uptake is concentration-dependent. Hepatic fatty acids are oxidized primarily in mitochondria, where they undergo complete oxidation to CO₂ via the citric acid cycle, or partial oxidation to produce ketone bodies. As discussed, plasma levels of the primary ketone body, β-HB, were elevated three-fold in HcB-19 (FIG. 2c). Consistent with these findings, a two-fold increase in ketone body synthesis was observed in liver slices of HcB-19 mice as determined by incorporation of ¹⁴C-oleate (FIG. 5e). In contrast, ¹⁴C-oleate incorporation into CO₂ was significantly decreased (FIG. 5J), demonstrating reduced oxidation by the citric acid cycle. Taken together, the above data indicate that the HYPLIP1 mutation results in increased FFA uptake by liver and decreased oxidation of FA by the citric acid cycle, resulting in increased FA availability for triglyceride and ketone body synthesis. Furthermore, plasma lactate levels were significantly increased in HcB-19 mice (FIG. 5g), while plasma pyruvate levels were decreased (FIG. 5h). The lactate/pyruvate ratio is reflective of the [NADH]/[NAD+] concentration (Williamson, et at., Biochem. J. 103:514-527 (1967)), thus, the increased lactate and decreased pyruvate likely reflects an altered redox state resulting from the HYPLIP1 nonsense mutation in Vdup1.

Human Vdup1 was first isolated from HL-60 cells stimulated to differentiate into monocytes/macrophages by 1,25-dihydroxyvitamin D₃ treatment (Chen et al., 1994, supra). In addition to up-regulation by 1,25-dihydroxyvitamin D₃, more recent work revealed both murine and human Vdup1 proteins bind to reduced thioredoxin (TRX) in vitro and in vivo and inhibit its reducing activity (Nishiyama et al., 1999, supra and Junn et al., 2000, supra). From the use of partial proteins, it was demonstrated that amino acids 134-395 of murine Vdup1 are required for TRX binding and inhibition (Junn et al., 2000, supra). Since the Vdup1 gene in HcB-19 contains a nonsense mutation at amino acid 97, the truncated protein will be missing these crucial amino acids, thus resulting in misregulation of thioredoxin.

Thioredoxin is a 12-kDa thiol oxidoreductase with many cellular functions, including cell activation (Yodoi, et al., Immunol. Today 13:405-411 (1992)), cell growth (Gasdaska, et al., Cell Growth Differ. 6:1643-1650 (1995)), apoptosis (Ueda, S. et al., J. Immunol. 161:6689-6695 (1998)), signal transduction (Nakamura et al., 1997, supra), and gene expression (Hirota, K. et al. Proc. Natl. Acad. Sci. USA 94:3633-3638 (1997)). Since Vdup1 binds and inhibits thioredoxin, the nonsense mutation in HcB-19 may cause hyperlipidemia by affecting the TRX pathway, one of the major reducing systems (Holmgren, J. Biol Chem. 264:13963-13966 (1989)). Alterations in redox state caused by misregulation of thioredoxin could explain several aspects of the hyperlipidemic phenotype. For example, increased [NADH]/[NAD+] has been demonstrated to inhibit the flux through the citric acid cycle and result in decreased CO₂ production (LaNoue, et al., J. Biol. Chem. 247:667-679 (1972) and Kimura, et al., Pediatr Res 23:262-265 (1988)). As a consequence, more fatty acids are available for utilization through the alternative oxidative pathway, ketogenesis, as well as for esterification and triglyceride synthesis. The increase in triglycerides and ketone bodies and decrease in CO₂ production observed in HcB-19 mice are consistent with this hypothesis.

These results provide evidence for a novel pathway with a profound influence on the regulation of lipid metabolism. The HYPLIP1 mutation may cause a decreased flux of FA through the citric acid cycle, resulting in increased FA availability for ketogenesis and triglyceride synthesis.

### EXAMPLE 6

Expression Profiling:

Expression profiling was performed on arrayed gene chips from Affymetrix (Santa Clara, Calif.) and Incyte Genomics (Palo Alto, Calif.). Samples were assayed by comparing three affected vs age matched control polyA RNA from the liver. Preliminary results suggest that oxidative stress markers seem to be predominantly upregulated whereas calcium binding proteins are down regulated in the Hyplip mice. A small sampling of genes from a much larger list that are up and down regulated is shown below:

| DIFFERENCE | ACCESSION | GENE_NAME (Up-regulated) |
|---|---|---|
| 10.4 | AA510289 | vh58f08.r1 Soares mouse mammary gland NbMMG Mus |

musculus cDNA

| 10.5 | AA175794 | ms95a05.r1 Soares mouse 3NbMS Mus musculus cDNA |
|---|---|---|
| 10.8 | U37527 | Mus musculus G protein gamma subunit mRNA |
| 11.9 | AF004105 | Mus musculus BM28 homolog mRNA |
| 13.5 | M27009 | Mouse alpha-1 acid glycoprotein (Agp-2) mRNA |
| 14.3 | X63023 | M.musculus mRNA for cytochrome P-450IIIA. |
| 14.4 | X81627 | M.musculus 24p3 gene. |
| 24.1 | W13166 | ma93f11.r1 Mus musculus cDNA |
| 25.6 | U38940Mus | musculus asparagine synthetase mRNA |
| 36.7 | U49430Mus | musculus ceruloplasmin mRNA |
| DIFFERENCE | ACCESSION | GENE_NAME (Down-regulated) |
| −97.5 | AA003244 | mg48g10.r1 Soares mouse embryo NbME13.5 14.5 Mus |

musculus cDNA clone 427056 5′ similar to 60S RIBOSOMAL PROTEIN L39.

| | | |
|---|---|---|
| −82 | U28486 | Mus musculus uterine-specific proline-rich acidic |
| | | protein mRNA |
| −81.1 | M12347 | Mouse skeletal alpha-actin gene |
| −63.1 | AF028071 | Mus musculus calcium binding protein D-9k mRNA |
| −58.1 | D14010 | Mouse reg I gene for regenerating protein I |
| −57.3 | M57590 | Mouse fast skeletal troponin C (sTnC) gene |
| −28.6 | M12347 | Mouse skeletal alpha-actin gene |
| −27.3 | J04992 | Mouse fast fiber troponin I mRNA |
| −22.8 | L49470 | Mus musculus troponin T fast skeletal muscle |

isoform FA5e17(Tnnt3) mRNA

### EXAMPLE 7

Cosmid Library:

Cosmid libraries were constructed for both C3H and HcB-19 mice strains with the use of pFOS1 vector. The goal is to sequence the entire “critical region” of 150 kb from the mutant mouse (HcB-19) and from the closest predecessor strain (C3H) to exclude conclusively the presence of mutations in addition to the stop codon mutation in the Vdup1 gene. Five cosmids from the C3H library and 5 clones from the HcB-19 library to cover the critical region were identified. These cosmids have been shotgun cloned into pUC 18 and are sequenced.

TABLE 1
Recombinant Mice
This table indicates the phenotypes and genotypes of mice derived from the original HYPLIP1 mutant mouse (HcBl9 or H19).
The table shows genetic and phenotype analysis of mice produced from an F2 cross (designated F2 or F2B).
These mice were investigated because they showed genetic recombination in the HYPLIP1 region and could therefore be used to further delimit the location of the HYPLIP1 gene.
The recombinant mice were mated back to a Castaneous mouse and progeny were generated (designated RP). Recombinant progeny mice were generated to estimate
the biological variability of the original parental mouse phenotype. Crosses indicated as RP2760, 2RP597, RP 1806, RP1950, or RP3003 are progeny from corresponding
F2 or F2B mice (2760, 597, 1806, 1950, and 3003). The animals were bled and two phenotypes, ketone and triglyceride (TG), were measured in the blood plasma. The
mice and their progeny were also genotyped with additional markers (columns D3Mit76 through D3Mit75). The “H19”, “H”, or “C” indicates that the particular
mouse (Column 1) is either homozygous for the HcB19 allele (H19), heterozygous, HcB19/Castaneous (H) or homozygous for the Castaneous allele (C). The genetic
recombination events were positioned between the two closest markers bounding the recombination event. The retention of the HcB19 genotype and the HcB19 phenotype
or the retention of the HcB19 genotype and the loss of the HcB19 phenotype could then be used to define the minimal region for the HYPLIP1 locus (shown in gray).

| Mouse_ID | Sex | Strain | Ke- tone | Tri- gly- cer- ides | D3Mit- 76 | 360- D18S | 418- F/G | AA- 259576 | 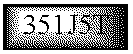 | 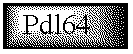 | 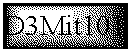 | Pds- 13 | D3- Pdm68 | D3- Pdm67 | D3- Pdm63 | D3- Pdm23 | D3- Pdm12 | D3Mit100 | D3Mit157 | D3Mit233 | D3Mit75 |
|---|---|---|---|---|---|---|---|---|---|---|---|---|---|---|---|---|---|---|---|---|---|
| 2187 | M | F2B | 44 | 147 | H | . | . | . | 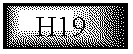 | 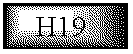 | 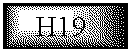 | . | . | H19 | H19 | H19 | . | H19 | H19 | H19 | H19 |
| 2187B | M | F2B | 44 | 147 | H | H | H/H | H197 | 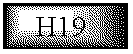 | 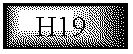 | 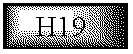 | . | . | H19 | H19 | H19 | H19 | H19 | H19 | H19 | H19 |
| . | . | . | . | . | . | . | . | . | 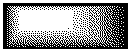 | 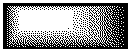 | 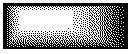 | . | . | . | . | . | . | . | . | . | . |
| 2760 | M | F2B | 9 | 124 | H19 | H19 | H19 | . | 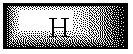 | 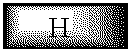 | 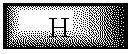 | . | H | H | H | H | . | H | H | H | H |
| 4318 | M | RP2760 | 14 | 94 | H19 | H19 | H/? | . | 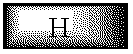 | 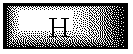 | 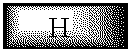 | . | . | H | H | H | . | H | H | H | H |
| 4319 | M | RP2760 | 11 | 141 | H19 | H19 | H19 | H19 | 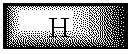 | 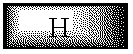 | 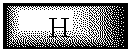 | . | . | H | H | H | . | H | . | H | H |
| 4320 | M | RP2760 | 42 | 175 | H19 | H19 | H19 | H19 | 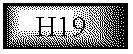 | 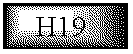 | 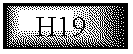 | . | . | H19 | H19 | H19 | . | . | . | . | . |
| 4321 | F | RP2760 | 60 | 99 | H19 | H19 | H19 | . | 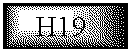 | 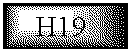 | 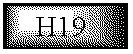 | . | . | H19 | H19 | H19 | . | . | . | . | . |
| 4322 | F | RP2760 | 20 | 6 | H19 | H19 | H19 | . | 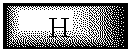 | 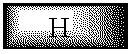 | 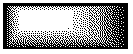 | . | . | H | H | | . | . | . | . | . |
| 4323 | F | RP2760 | 52 | 132 | H19 | H19 | H19 | . | 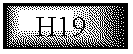 | 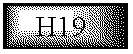 | 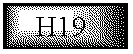 | . | . | H19 | H19 | H19 | . | . | . | . | . |
| 4324 | F | RP2760 | 55 | 199 | H19 | H19 | . | . | 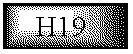 | 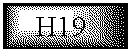 | 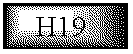 | . | . | H19 | H19 | H19 | . | . | . | . | . |
| 4325 | M | RP2760 | 19 | 213 | H19 | H19 | . | . | 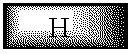 | 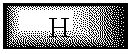 | 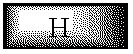 | . | . | H | H | H | . | . | . | . | . |
| 4326 | M | RP2760 | 18 | 96 | H19 | H19 | H19 | . | 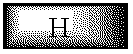 | 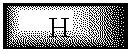 | 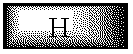 | . | . | H | H | H | . | . | . | . | . |
| 4327 | M | RP2760 | 21 | 167 | H19 | H19 | H19 | . | 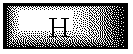 | 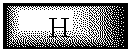 | 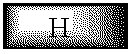 | . | . | H | H | H | . | . | . | . | . |
| . | . | . | . | . | . | . | . | . | 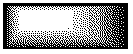 | 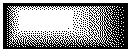 | 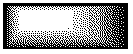 | . | . | . | . | . | . | . | . | . | . |
| 2811B | F | F2B | 51 | 191 | H | H19 | H19 | . | 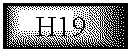 | 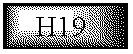 | 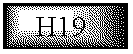 | . | . | H19 | H19 | H19 | H19 | H19 | H19 | H19 | H19 |
| . | . | . | . | . | . | . | . | . | 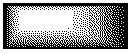 | 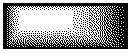 | 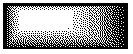 | . | . | . | . | . | . | . | . | . | . |
| 3085B | M | F2B | 6 | 16 | H | H19 | H19 | . | 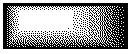 | 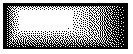 | 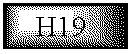 | . | . | H19 | H19 | H19 | H19 | H19 | H19 | H19 | H19 |
| . | . | . | . | . | . | . | . | . | 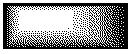 | 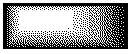 | 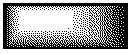 | . | . | . | . | . | . | . | . | . | . |
| 597 | F | F2B | 34 | 53 | h19 | . | . | H19 | 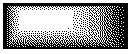 | 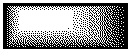 | 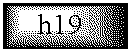 | . | . | . | . | . | . | C | C | C | C |
| 2638 | F | 2RP597 | 38 | 364 | . | . | . | H19 | 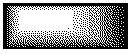 | 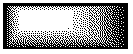 | 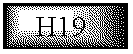 | . | H | C | . | . | . | . | C | C | C |
| 2639 | F | 2RP597 | 44 | 265 | H19 | . | . | H19 | 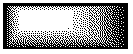 | 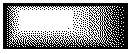 | 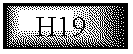 | C | . | C | . | . | . | C | C | C | C |
| 2640 | M | 2RP597 | 52 | 322 | H19 | H19 | . | . | 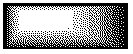 | 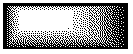 | 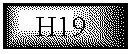 | . | H | H | H | H | H | H | H | H | H |
| 2641 | M | 2RP597 | 36 | 424 | H19 | . | . | . | 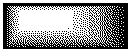 | 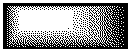 | 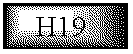 | C | H | C | C | C | C | C | C | C | C |
| 2644 | M | 2RP597 | 47 | 310 | H19 | H19 | . | H19 | 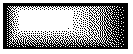 | 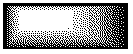 | 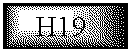 | . | H | H | H | H | H | H | H | H | H |
| 2645 | M | 2RP597 | 26 | 480 | H19 | H19 | . | H19 | 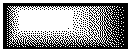 | 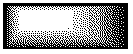 | 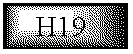 | . | H | H | H | H | H | H | H | C | C |
| 2642 | M | 2RP597 | 52 | 373 | H19 | . | . | H19 | 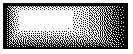 | 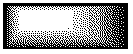 | 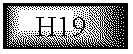 | . | H19 | . | . | . | . | H19 | H19 | H19 | H19 |
| 2643 | M | 2RP597 | 26 | 756 | H19 | . | . | H19 | 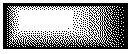 | 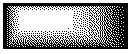 | 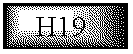 | . | H19 | . | . | . | . | H19 | H19 | H19 | H19 |
| . | . | . | . | . | . | . | . | . | 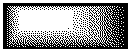 | 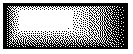 | 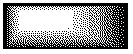 | . | . | . | . | . | . | . | . | . | . |
| 1806 | . | F2 | . | . | . | . | . | . | 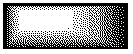 | 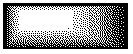 | 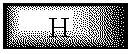 | . | H19 | H19 | H19 | H19 | . | . | . | . | . |
| 5057 | M | RP1806 | 62 | 569 | H19 | . | . | . | 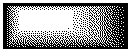 | 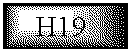 | 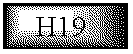 | H19 | H19 | H19 | H19 | H19 | H19 | H19 | H19 | H19 | H19 |
| 5058 | M | RP1806 | 15 | 145 | H | . | . | . | 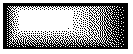 | 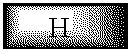 | 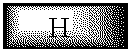 | H | H19 | H19 | H19 | H19 | H19 | H19 | H19 | H19 | H19 |
| . | . | . | . | . | . | . | . | . | 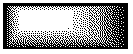 | 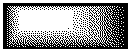 | 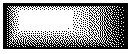 | . | . | . | . | . | . | . | . | . | . |
| 1950 | M | F2B | 7 | 15 | C | . | . | C | 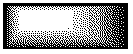 | 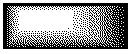 | 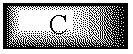 | . | H | H | H | . | . | H | H | H | H |
| 4246 | F | RP1950 | 21 | 112 | H | . | . | . | 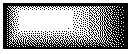 | 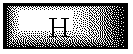 | 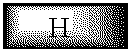 | . | . | H19 | H19 | H19 | H19 | H19 | H19 | H19 | H19 |
| 4254 | M | RP1950 | 21 | 152 | H | . | . | . | 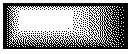 | 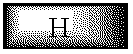 | 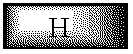 | . | . | H19 | H19 | H19 | H19 | H19 | H19 | H19 | H19 |
| 5003 | F | RP1950 | 26 | 99 | H | . | . | . | 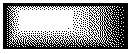 | 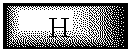 | 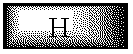 | H19 | H19 | H19 | H19 | H19 | H19 | H19 | H19 | H19 | H19 |
| 5004 | F | RP1950 | 28 | 136 | H | . | . | . | 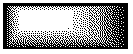 | 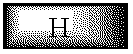 | 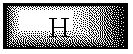 | H19 | H19 | H19 | H19 | H19 | H19 | H19 | H19 | H19 | H19 |
| 5008 | M | RP1950 | 21 | 90 | H | . | . | . | 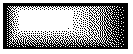 | 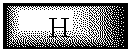 | 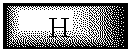 | . | H19 | H19 | H19 | H19 | H19 | H19 | H19 | H19 | H19 |
| 5009 | M | RP1950 | 29 | 125 | H | . | . | . | 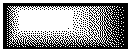 | 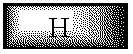 | 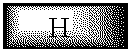 | . | H | H | H | H | H | H | H | H | H |
| 5010 | M | RP1950 | 24 | 257 | H | . | . | . | 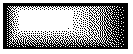 | 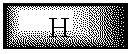 | 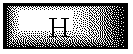 | . | H19 | H19 | H19 | H19 | H19 | H19 | H19 | H19 | H19 |
| 5011 | M | RP1950 | 29 | 306 | H | . | . | . | 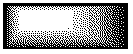 | 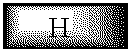 | 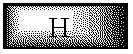 | . | H | H | H | H | H | H | H | H | H |
| . | . | . | . | . | . | . | . | . | 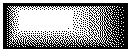 | 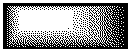 | 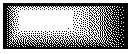 | . | . | . | . | . | . | . | . | . | . |
| 3003 | F | F2B | 3 | 12 | H | . | . | . | 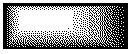 | 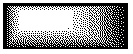 | 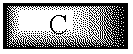 | . | . | . | . | . | . | C | . | C | . |
| 4262 | F | RP3003 | 53 | 406 | . | . | . | H19 | 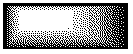 | 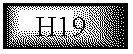 | 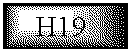 | . | H | H | H | . | . | . | . | . | . |
| 4263 | F | RP3003 | 19 | 115 | H | . | . | . | 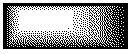 | 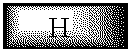 | 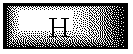 | . | H | H | H | H | H | H | . | . | . |
| 4264 | F | RP3003 | 44 | 425 | H19 | . | . | H19 | 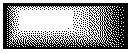 | 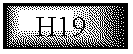 | 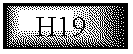 | H | H | H | H | H | . | . | . | . | . |
| . | . | . | . | . | . | . | . | . | 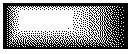 | 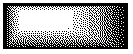 | 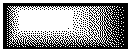 | . | . | . | . | . | . | . | . | . | . |
| 3284 | M | F2 | 19 | 75 | H | H | . | . | 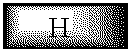 | 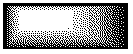 | 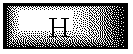 | . | H | C | C | C | C | C | C | C | C |
| . | . | . | . | . | . | . | . | . | 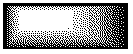 | 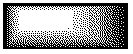 | 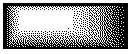 | . | . | . | . | . | . | . | . | . | . |
| 3442 | M | F2 | 60 | 227 | H19 | . | . | . | 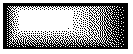 | 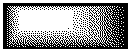 | 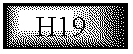 | . | H19 | . | H | H | H | H | H | H | H |

TABLE 2
Select informative recombinant animals and their backcross progeny

| | Ket. | TG | | | Pred. | | Ave. | Ave. | |
|---|---|---|---|---|---|---|---|---|---|
| ID | mg/ | mg/ | Crossover | Geno- | Prob. | # of | Ket. | TG | Ave. |
| # | dl | dl | Breakpoint | type | P(c/h) | Prog. | mg/dl | mg/dl | P(h/h) |
| 1 | 82 | 1063 | D3Pds1-D3Pds3 | c/h-h/h | 0.00001 | 15 | 53 ± 3 | 339 ± 51 | 0.924 |
| 2 | 44 | 218 | D3Pds1-D3Pds3 | c/h-h/h | 0.056 | 11 | 52 ± 3 | 378 ± 45 | 0.975 |
| 3 | 54 | 980 | D3Pds1-D3Pds3 | c/h-h/h | 0.012 | 3 | 53 ± 3 | 157 ± 37 | 0.962 |
| 4 | 17 | 27 | D3Pds1-D3Pds3 | c/c-c/h | 0.635 | 7 | 56 ± 3 | 276 ± 50 | 0.975 |
| 5 | 6 | 98 | D3Pds1-D3Pds3 | h/h-c/h | 0.994 | 1 | 23 | 240 | 0.152 |
| 6 | 26 | 19 | D3Pds1-D3Pds3 | h/h-c/h | 0.966 | 4 | 17 ± 4 | 118 ± 20 | 0.058 |
| 7 | 25 | 28 | D3Pds1-D3Pds3 | h/h-c/h | 0.961 | 6 | 22 ± 2 | 86 ± 23 | 0.094 |
| 8 | 18 | 36 | D3Pds1-D3Pds3 | h/h-c/h | 0.978 | 2 | 22 ± 3 | 70 ± 5 | 0.051 |
| 9 | 8 | 71 | D3Pds1-D3Pds3 | c/h-c/c | 0.575 | 15 | 13 ± 1 | 142 ± 21 | 0.023 |
| 10 | 44 | 147 | D3Pds7-D3Pds5 | c/h-h/h | 0.080 | 0 | — | — | — |
| 11 | 9 | 124 | D3Pds7-D3Pds5 | h/h-c/h | 0.987 | 6 | 17 ± 2 | 120 ± 29 | 0.064 |
| 12 | 7 | 15 | D3Mit101-D3Pds13 | c/c-c/h | 0.464 | 6 | 24 ± 1 | 141 ± 25 | 0.156 |
| 13 | 3 | 12 | D3Mit101-D3Pds13 | c/h-c/c | 0.383 | 2 | 49 ± 4 | 416 ± 10 | 0.959 |
| 14 | 34 | 53 | D3Mit101-D3Pds13 | c/h-c/c | 0.816 | 70 | 51 ± 1 | 305 ± 15 | 0.946 |
| 15 | ND | ND | D3Pds13-D3Pd168 | c/h-h/h | — | 6 | 20 ± 1 | 88 ± 13 | 0.062 |
| 16 | 60 | 227 | D3Pd170-D3Pd171 | h/h-c/h | 0.003 | 0 | — | — | — |
| 17 | 40 | 470 | D3Pd167-D3Pd163 | h/h-c/h | 0.058 | 0 | — | — | — |
| 18 | ND | ND | D3Pd167-D3Pd163 | c/h-c/c | — | 12 | 52 ± 3 | 501 ± 93 | 0.949 |
| 19 | 22 | 97 | D3Pd123-D3Pd112 | c/h-c/c | 0.773 | 5 | 51 ± 4 | 294 ± 42 | 0.960 |
| 20 | 9 | 133 | D3Mit100-D3Mit157 | c/h-h/h | 0.986 | 8 | 19 ± 3 | 137 ± 36 | 0.075 |
| 21 | 49 | 207 | D3Mit100-D3Mit157 | h/h-c/h | 0.024 | 6 | 38 ± 4 | 365 ± 92 | 0.773 |
| 22 | 19 | 49 | D3Mit100-D3Mit157 | c/h-c/c | 0.701 | 40 | 49 ± 2 | 405 ± 29 | 0.890 |
| 23 | ND | ND | D3Mit100-D3Mit157 | c/h-c/c | — | 8 | 60 ± 3 | 468 ± 63 | 0.995 |

The ketone body value (Ket.), triglycerides (TG), breakpoint, genotype, predictive probability of heterozygosity [P(c/h)], and number of recombinant backcross progeny are given for each recombinant animal. The average±s.e.m. (Ave.) ketone bodies, triglycerides, and predictive probabilities of being homozygous for HYPLIP1 [P(h/h)] are listed for all backcross animals that inherited the recombinant chromosome. Abbreviations: ND=not determined, h/h=homozygous HcB-19 alleles, c/c=homozygous CAST/Ei alleles, c/h=heterozygous.

### EXAMPLE 8

FCHL1 Patient Study:

Samples of 13 additional Dutch patients are sequenced for up to 2.2 kb of upstream promoter sequences. The same region for the original 53 samples are also sequenced.

### EXAMPLE 9

Physiologic and Pathologic Experiments:

As the human disease is not as severe as that seen in the Hyplip animals, it was suggested that the Vdup1 heterozygotes should be challenged with a HF diet to determine if these animals have a milder Hyplip phenotype. Depending upon the observed phenotype, additional animals can be used instead of or in parallel with the Vdup1 homozygotes. The first experiment examines the pathology of the wild type and homozygotes when fed a HF diet. The goal is to determine why the Hyplip animals die on the HF diet and what metabolic pathways are affected in the mutants. Two animals are needed for each time point (0, 5 days, 2 weeks). Parameters to be measured include blood levels of lactate, beta-hydroxy butyrate, acetoacetic acid, bicarbonate and blood gases. In addition to tissues harvesting for necropsy, portions of the livers will be harvested for analysis of protein content/enzyme activities for DGAT (diacylglycerol aclytransferase, which adds a fatty acid to diacylglycerol to a triacylglycerol), ACAT (acylcholesterol acyltransferase, which adds a fatty acid to free cholesterol to a cholesterol ester as the major storage form of cholesterol), microsomal lipase, triglyceride biosynthesis, Apo B, thioredoxin reductase. RNAs are prepared from these samples for Affymetrix RNA profiling. Since Vdup1 is believed to function in maintaining cellular redox potentials, it has been suggested that mitochondrial redox potential also be measured in liver samples. Additionally, beta oxidation rates in the animals are also evaluated.

### EXAMPLE 10

Primers developed for the FCHL1 locus are:

| | | | | SEQ |
|---|---|---|---|---|
| Exon | Forward | Reverse | bp | ID NO: |
| Exon 0 | GGGTCAGTGGGATCCT | CTGGTTACACTAAGCG | 409 | 6-7 |
| | CCTTC | AAGTC | | |
| Exon 1 | GGAGTGCTTGTGGAGA | CTCTAATCAGCTTTCA | 470 | 8-9 |
| | TCGG | CCCTC | | |
| Exon 2-3 | GCATGCCACCCAAGCA | ATCATCCTCATGACCC | 468 | 10-11 |
| | TTCC | AGGAG | | |
| Exon 4-5 | GACGTCTTGGGCATTA | CTGCTGAAGCCACCCT | 576 | 12-13 |
| | GATTG | GCATC | | |
| Exon 6-7 | GCTGTCTTGTTTCTCC | CAATCCGACTAAGTCA | 545 | 14-15 |
| | AGACC | ACTTC | | |
| Exon 8-A | GAAGTTGACTTAGACG | CAAGGTCCAAAGTTCC | 512 | 16-17 |
| | GATTG | TATCAC | | |
| Exon 8-B | CGATAGTTTCGGGTCA | TCCAAGGACTTACATG | 427 | 18-19 |
| | GGTAA | AAGAG | | |
| Exon 8-C | GCAGTAGTAACTGCCC | CATTAGTGTGGATTGT | 850 | 20-21 |
| | CACCA | TAAGTAGAGG | | |
| Ex8BR2 | GTGCTAAGAACATCAC | | | 22 |
| | CTTAGAATC | | | |

Primers developed for the HYPLIP1 locus are:

| | | | | SEQ |
|---|---|---|---|---|
| | | | | ID |
| Primer | Forward Pirmer | Reverse Primer | Size | NO: |
| TIF28F | GAACCCACTCGGCTCA | | 438 bp | 23 |
| | ATC | | | |
| TIF222F | GGTCTCAGCAGTGCAA | | 500 bp | 24 |
| | ACA | | | |
| TIF465R | | TTTGCCTCTTGAGTTG | 625 bp | 25 |
| | | GCTG | | |
| TIF631F | CTCCATCCATGCTGAC | | | 26 |
| | TTTG | | | |
| TIF721R | | GCCATTGGCAAGGTAA | | 27 |
| | | GTGTG | | |
| TIF1255R | | GATGCTTCATTTCCTG | | 28 |
| | | CAGC | | |

Additional primers for the HYPLIP1 locus (SEQ ID NOs: 29-406) are:

| oligo name | PCR primers |
|---|---|
| PIAS3 set 1 | f1-TGAGTTTCCGAGTGTCTGAGC |
| | r1-CCAGCAGCTCGTGTTTCCGCC |
| | f2-GATGAGTTTCCGAGTGTCTGAGCTCCAG |
| exon 1 | f1-CAGCGTCCAGATGAAGATCAAAG |
| | r1-CTAGACTTGAGGAGGTGCAG |
| exon 2 | f1-ACCTCAGAAAACTGCCACTTAC |
| | r1-CATTTATCCGCTGCCAAAGGG |
| exon 3 | f1-GAAGCCACTGCCCTTCTATG |
| | r1-TGGCTTCATGGTGACATCGG |
| exon 4 | f1-GGTGCTGAGTCAAGACAACAC |
| | r1-TCACTCACCTCAGAACCACCA |
| exon 5 | f1-CTTGCTGACCCCTAGCCCTG |
| | r1-GAAACCCTCTTCAGAATGTAAC |
| exon 6 | f1-TTCTGAAGAGGGTTTCTCTGG |
| | r1-GGAAGCTGAAGAGCCCTAC |
| exon 6a | r1-AGAGCTTCAGAAAGCTCAGGC |
| | f1-TTTTGAGGTCCCTGGATGTAC |
| exon 7 | f1-CAGCCTCGTGAATCCACATG |
| | r1-GACTTAGCTCAATTGGGGAGC |
| exon 8 | f1-CCTTTCTCTGAGACAGCCTTC |
| | r1-CTTCTGTCCCTCTGCTATGG |
| exon 9 | f1-TCCACTCACAGAGCCCTGAC |
| | r1-TCCACCTTAGGGTCAGTGTTC |
| exon 10 | f1-AAGGAGTCAGAAGGGAGTTGATT |
| | r1-GACTCTTCCGAGATCTTGGATC |
| univ. rev | r1-CTTTCCTTCAGACTGAGAGTCAG |
| | r2-TACATGAGTACCCACCTGCCTTC |
| | r3-GAGCTCCCAGGAGCCACAATGCTGCTG |
| | r4-GACGTCTGACCGACAGCCAGTCAAAG |
| pex 11-set 1 | f1-GGTCACAGGCATTTCTGAGCAC |
| | r1-CCAGGAGGAAGTCTGCCACAA |
| pex 11-set 2 | f2-GAGGCTCAGATGACTCTCCAG |
| | r2-CAGTATGCCTGTTCCCTTCT |
| HHCPA78 set 1 | f1-GAACATCACCTTAGAATCAATTCC |
| | r1-AGAAAGGACCTTCCACGGCTG |
| HHCPA78-set 2 | f2-GTACGCACTGCACGTTGACT |
| | r2-CGAGCTCGGTACCCCTC |
| HHCPA78-set 3 | f3-AACCTATCCGGCTGCAGC |
| | r3-GGCAACCCATCTACAAAACTC |
| hhcpa78.1 | ACCCTTCACAGGATTAGTGTTAG |
| hhcpa78.2 | CCCAACACTGGACAGGAAATGG |
| hhcpa78.3 | GCTTCATTTCCTGCAAGGCTC |
| hhcpa78.4 | GGTTTTCAAGCCCTACTTTTACGG |
| hhcpa78.5 | CTTGCGCTATGAAGACACACTTC |
| hhcpa78.6 | GACTACTGGGTGAAGGCTTTTC |
| hhcpa78.7 | CTCAGAAGCTGTCCTCAGTCAG |
| hhcpa78.8 | CCAGCCGTGGAAGGTCTTTTC |
| aa414944 | f1-TTCTAGACCAGCTGAGACATC |
| | r1-TCCATGGCGAAATCTTCGCC |
| aa271906 | f1-GACTTCTTGCTAAGACCAGATAC |
| | f2-CTCATACTGTGAGGACTTGATG |
| | r1-CTCGGCTGCAGATTCTTGTAC |
| aa278124 | f1-AAGTGCCATCCTGCTTCCG |
| | r1-ACAGACGTTGGGTCACCAC |
| | f2-AACAGCTATGACCCATGATTACG |
| | r2-GGTGACTTCACTCAGGTTTC |
| | f3-CTGGTGGAAGACAACAACCG |
| | r3-AGCTATGACTTGGGGACTCC |
| | f4-GTCATAGCTCTCACTCATGTCC |
| | r4-CTCCCACGCATGCTTATGTATG |
| D3pdlmg1 | CTCATGTATGGCCTCTTGTATG |
| | CCCACTACTGTTCCTTGTCTG |
| D3pdlmg2 | CCAGGGATCTGATGCTTTCTTC |
| | TCAAACAAGATAGTCCCGTAAAG |
| D3pdlmg3 | CAAAAAGATAGACAGGCAGGCAG |
| | TTCTGGTCTTCATGGGCACTG |
| D3pdlmg4 | CAGACATTTGCACACACTTGTGC |
| | CTGGCCTTGAACTCAGAGATTG |
| D3pdlmg5 | CTACTCAGAAGGTTGAGGCAG |
| | GCATCAGAAAGTGGGACCTC |
| D3pdlmg6 | GAATGCTGAAGTTCTGCACGTG |
| | AGTCCACTCGCTACTCAGAAG |
| D3pdlmg7 | CTTTTGGGGAGTTTCCTTCTGG |
| | AACAGGAGTCCCTGAAGAATGC |
| D3pdlmg8 | CTCAGCTGCATAGCAAGTTCC |
| | TTGAAGTCCTTAGGGTGCGTC |
| D3pdlmg9 | CTCTTCTGGTTTAGGCCATAAC |
| | GTACAGTAGCCTGCGTTGTTAG |
| D3pdlmg24-f | GAGGATCCCCCTGTAAGAG |
| D3pdlmg24-r | GGAAACAGCTATGACCATGGCCTGGCAAAAGCAT |
| D3pdlmg25-f | GCATGCCTGCAGGTCGAC |
| D3pdlmg25-r | GGAAACAGCTATGACCATGTGTTAGACACGATACACC |
| D3pdlmg26-f | GTAAAACGACGGCCAGTGACCTAAACTGGGG |
| D3pdlmg26-r | GAGCATTTGAGCATTTGCCTAG |
| D3pdlmg27-f | GTAAAACGACGGCCAGTTTGAACTGTGAGG |
| D3pdlmg27-r | CTGATGAGAAACCATCCAAAAC |
| D3pdlmg28-f | GTAAAACGACGGCCAGTGCTTGGGAGGTGG |
| D3pdlmg28-r | GACTCCTCATCATACTGCTGC |
| D3pdlmg29-f | GTAAAACGACGGCCAGTGCAGGACGTTTC |
| D3pdlmg29-r | GTCAGTCTTATCCGCTGAGC |
| D3pdlmg30-f | GTAAAACGACGGCCAGTAAGAGGGGCATGTGG |
| D3pdlmg30-r | CATATGCAGAACAGGCTGG |
| D3pdlmg31-f | GAGTAGCTACCTTGGCAC |
| D3pdlmg31-r | GGAAACAGCTATGACCATGTTTTGTCTGTATGTAT |
| D3pdlmg32-r | GAAGTCCTGTCTTAGAAACAAC |
| D3pdlmg32-f | GGAAACAGCTATGACCATGGTTGCCTTACCTGG |
| D3pdlmg33-r | GGAGACAGAAGACCTGCCC |
| D3pdlmg33-f | GGAAACAGCTATGACCATGATGGAGAGGAAC |
| D3pdlmg34-r | CAAAGCTAAACCAGGTGAAACTG |
| D3pdlmg34-f | GGAAACAGCTATGACCATGGCTACAGAGGTTTTC |
| D3pdlmg35-r | CCTTCATTTCTTGGAACTAAGC |
| D3pdlmg35-f | GGAAACAGCTATGACCATGTGGTTGCTAGAAAC |
| D3pdlmg36-r | GTAAAACGACGGCCAGTTCGGAGACCCTCATAC |
| D3pdlmg36-f | GTTCATGGCTAGCCTGGTCTAC |
| D3pdlmg37-r | CTGCTTCCACACATGCTAGAG |
| D3pdlmg37-f | GGAAACAGCTATGACCATGGCTGTATAGATATTTTAT |
| | TG |
| D3pdlmg38-r | CTTGGTAGCCATTGTTGGATTAG |
| D3pdlmg38-f | GGAAACAGCTATGACCATGATAAAAACCAACCTGGGG |
| D3pdlmg39-r | GTAAAACGACGGCCAGTGCTCACAGGACAGAC |
| D3pdlmg39-f | CCAAAGTTTATGTAGCTAGG |
| D3pdlmg40-r | GCCCATTCTCAGGACGAAG |
| D3pdlmg40-f | GGAAACAGCTATGACCATGATGACATTGGTATAAC |
| D3pdlmg41-r | GTAAAACGACGGCCAGTCCACTCGCTACTCAGAAG |
| D3pdlmg41-f | GCCTTAGACCAGAGCAGGACT |
| D3pdlmg42-r | GTAAAACGACGGCCAGTTCCAGGTCAGCCTGG |
| D3pdlmg42-f | CTTACTTGGCCTGGATCCTG |
| D3pdlmg43-r | GTAAAACGACGGCCAGTGGTGGAGAAGAGC |
| D3pdlmg43-f | CAAAAAGCCAAGGGGCTTCTC |
| 354.1f | TTGGTTTCTAATACAGTGCTTCAGAG |
| 354.1r | CAAAGAGGAACTATTTCAGACTTGGT |
| 354.2f | CTTGTTTTTAAACCTTGAGGTCAGAT |
| 354.2r | TGCCCTGTTTTTGTTTATAAGACTTT |
| 354.3f | GACAGGCCTAGACAGTTCTTACTCTT |
| 354.3r | CTGTCTAGTCTTCAGACTGGTTCTTG |
| 354.4f | ACTCTTTCCTAAACAGTCCATCAGTT |
| 354.4r | ACACCAGATCCATTCTCATGTTATAG |
| 354.5f | CTTATGAATCAGGAATTATGCTCCTT |
| 354.5r | TTTTTATTTTATGGGTATGGGTGTTT |
| 354.6f | TTGATGGTGTCCTTATTTACAAAGAG |
| 354.6r | TTCTATACTGGACACGCAACTTACTC |
| 354.7f | CTCTTTATAGGACATATGTGCAAAGC |
| 354.7r | TCATCCTAAGTCTTAAAAAGGAGACA |
| 354.8f | CTCTTTATAGGACATATGTGCAAAGC |
| 354.8r | TCATCCTAAGTCTTAAAAAGGAGACA |
| 354.9f | CACAGAGATCTACCTGTCTCTGTCTC |
| 354.9r | GAACTAGGATGTTTCAGACAGTGTTC |
| 354.10f | CATCGTAGTTAATTGGTCATCTGAGT |
| 354.10r | GAGATACAAAGCTGTTGTGTGATTG |
| 354.11f | TAGATAAAGCAGGGTCTCTTGTAACC |
| 354.11r | TAGTAGTCATTTTATTGCCATGTTCC |
| 354.12f | TCTAAGACTCTGGAAGCTACAACGTA |
| 354.12r | GTAAATTAGCTCCGTGGAAAATAAAG |
| 354.13f | TTTCAGAGTGTTTTCAGAGAAAAGTG |
| 354.13r | TATTGGTTTTTGTTTGTTTGTTTGTT |
| 354.14f | ACAAAACAAGACAGCCTACACAATAG |
| 354.14r | ATTCTTGAGTTTCTCATTTCCAGTCT |
| 354.15f | ATTAGGAATGTAAGGAAGGTGGTTTT |
| 354.15r | CATCAATAGCTAATAAAAGTGGGTGA |
| 354.16f | ATTAGGAATGTAAGGAAGGTGGTTTT |
| 354.16r | CATCAATAGCTAATAAAAGTGGGTGA |
| 354.17f | AGCTACACAGAGAGACAGACACCTTA |
| 354.17r | TAAGACATGGAAAATGCCTTTATGTA |
| 354.18f | TTACATTCATGCTCCTAGCTTCTTTA |
| 354.18r | AGGATGTATTTCACTATGTGGTTCAG |
| 354.19f | GGAGTGTTCTCCATCTCTCTTTTAAC |
| 354.19r | GGCATCTATAAGACCCACAAAAATAA |
| 354.20f | AAGAAGGAGAATGCCTACAACACTA |
| 354.20r | GCTAAATTCAAACTCTAACAGGAAGC |
| 354.21f | GACATGAAATAAAAATGCCTCAAAC |
| 354.21r | GTAAAGAAGCCAAGCTATAGAGGAAA |
| 354.31f | TTGTAACACACACACACAAATGCACA |
| 354.31r | CACTGTCACTGTCTTCAGACACACCA |
| 354.32f | GAACAGCCTCAAGTTAGGGGTGAGAT |
| 354.32r | CCTCCCACTGGCAGCTAAAAATAAAG |
| 354.33f | CAGTCAAGTTGTGACTCTGTGCCAGT |
| 354.33r | AGGTTGTTCTCTGACCCGCATATACA |
| 354.34f | AGAACATGACAGGAGACAGGAAGGTG |
| 354.34r | GAGACCCAGATCTGTGACCTTTGTTC |
| 354.35f | AACCATCACAGTGTCCCTTCTTAGCA |
| 354.35r | TGACTTCAGTTCGGAGACCCTCATAG |
| 354.36f | CCCTGTGATCTGGATGGAATACTGAC |
| 354.36r | CTTTGCAACCTGTCCCTGTAAAACTG |
| 354.37f | GCCATGTTTCCTAAAAGGCCTAGAGA |
| 354.37r | GCAGGTAGGAGGATCAGGAAATCAAG |
| C4a-f | CACCTGCATTCACACACACA |
| C4a-r | CTGAGAACTGTGCAGCCAGA |
| C4b-f | CCAGGAAACAAGAGGTGGAC |
| C4b-r | GGCATGCAATCGCTTTTTAC |
| C4c-f | GAGAGCAGGGCTATGTGCTT |
| C4c-r | GTCACCACAAAGGCTCCAGT |
| C4d-f | CCTCCAAAGTGCTGGGATTA |
| C4d-r | ACTCCACCCCAGTGAAACAG |
| C4e-f | TGTCTTCTCAAGAGACAGAGCTT |
| C4e-r | ATTTTCTCTCTGCCGCTCAG |
| C4f-f | TCCTGACAGTTTCCCCATTC |
| C4f-r | AGGCAGGCAGATTTGTGAGT |
| C4g-f | CCAATCCCAAAACTTGGAAA |
| C4g-r | GGTGTGCTATTTGGGGTGTT |
| C4h-f | ACGATCCCTCTCACAGGATG |
| C4h-r | GGTCAAGGCCAGAACTTCAT |
| C4i-f | ACGATCCCTCTCACAGGATG |
| C4i-r | GGTCAAGGCCAGAACTTCAT |
| C4j-f | CAGAGCCAGCTCTACCCACT |
| C4j-r | AAGCCTCATTTGCATGTTCC |
| C4k-f | AAAGATCACGATGGCCTGTT |
| C4k-r | TTTGGTTCCATTCCAGCATT |
| C4l-f | ATCTACGCGCCTGAGAAGAC |
| C4l-r | AGGGGTTGGTAGGAACTGGT |
| C4m-f | CAAACACTTCGGGAGCAGAG |
| C4m-r | TGGTGCACTTCTTCCATTTG |
| C4n-f | ATAAAGGCGGCAAGGTTTTT |
| C4n-r | CCGTCCCTCTTAAGGAGCAT |
| C4o-f | CAGGGTTGCTCTGGGTAAAA |
| C4o-r | GAAGTGCGTTCCCTTTGTGT |
| C4p-f | CATTTTCCTGTCAGCAACCA |
| C4p-r | CTGCATAGGTCCCAGTGTCC |
| C4q-f | TGAGCAGCCACTCTAGTCCA |
| C4q-r | CCAGTGTAGGATGGGCATGT |
| C4r-f | GAGGAGGTGCATCAGAGGAC |
| C4r-r | TAAAGGGCCGTTGAGACCTT |
| C4s-f | TCTGAGAGGGAGGGAACCTT |
| C4s-r | ACCCTTCCATGTCACTGCTC |
| C4t-f | TCCTTTACTGTGCCCCCTTT |
| C4t-r | TCCAGGACCTGCCTTATTTG |
| C4u-f | GCTCGCCGTGATCGTATAGT |
| C4u-r | TCAAAGCAAAGCAAAGCAAA |
| a integrin 10 | f1-GGCCGAATTTGGATACAGTGTC |
| | f2-GCTCCTCCTTTAATCTGGATGA |
| | f3-GAGAACCCTGTGCATGAGTG |
| | f4-GTATCATGGAACCCAGACTGG |
| | r1-CTTGAGATAGTGACAGGAAGTAG |
| | r2-CATTGTGAACCAGCCTCAGC |
| aa049675 | f1-TGAATGGCCCTTCCCTATGGT |
| | r1-CTTGCATGCCTGCAGGTCGA |
| RPC 62 | f1-GGAAAACCCCTAAGTACATAGGG |
| | r1-GACAATGGAGAGGACTTTGGAG |
| aa032381 | f1-GAACTGGCCTAGATGGCTGC |
| | r1-CCTGAGAACCCCTATGTTTAG |
| aa051102 | f1-ACTAGAAAAGTCTCAGCGGG |
| | r1-GCGTCCAACTTGTTAACGTTG |
| aa387373 | f1-TACCTCGGACACGGGATCTC |
| | r1-GCCAGGCATGGAGAAGCAAG |
| RBM8 | f1-CTTGAAGGGGTATACTCTAGTTG |
| | r1-CCTAGCCCTGTTTATCCAAGG |
| aa414944 | f1-TTCTAGACCAGCTGAGACATC |
| | r1-TCCATGGCGAAATCTTCGCC |
| w13578 | f1-ATTTTCCTGGGACGTCACCC |
| | r1-GCAGAGCAGCTGTTTCCTTC |
| | r2-GACACAGACTCCAGCTTCAG |
| aa104103 | f1-CTCTGTGAGTTTGAGGCCAG |
| | r1-GGTAACAGGAGCAGATCAGG |
| aa116392 | f1-GGGTACAGAGATGCCAGTTA |
| | r1-GTCTGGAGTTCGTGTGTCCT |
| STS? | f1-CTGCAGTGAGTTTTTCAGCCC |
| | r1-CATTTGCTCACCATGACAGTCC |
| musgs01617 | f1-GACATGTTAGACAGATACAAACATTTT |
| | r1-CAGCAGACTTACTAGCATTTACT |
| w35051 | f1-AGAGGAGGCGGCGACGGCGGT |
| | r1-ACCGGTAGTTTGGGATTGACC |
| aa052450 | f1-GTGCTTAGGAGAGGATTTCATG |
| | r1-CGAGTTCGAGTCCCAGAACC |
| aa755852 | f1-GCGTACGTGCAGGCAAAATAC |
| | r1-CATCTGCCAGAATTCCTGAAGGG |
| ai099193 | f1-GCCAAAATGACAAACACAAAGGG |
| | r1-GTCTCTGCTCTTTGAGTGCTTG |
| w40907 | f1-CTGCGCTTCCACAAGAGAAAG |
| | f2-GCGAAGTGCTGCAAAGATTGC |
| | r1-TATACAGAAAATAT5CATCCTAAGTC |
| 126O21S | ATGGATTGGAGAGGCTGATG |
| | CCAGACTCACATTGCCAGAG |
| 148H23T | CAGGCTGTTTGAGGCTTCTC |
| | TGTGGGCAAGGACCAGTTAT |
| 152O1T | TGTACAAAACGGAGCCAAAA |
| | CTAGTCCAGGCTCCACAAGC |
| 184N22T | ATTGCCTTCCTTGTTGCCTA |
| | GCAAACCTCTCCTTATTGTGTTC |
| 186D21S | CAGGCTAACCCCAAACTCAG |
| | AAACAGGTCCCAAGGAATCA |
| 189C4S | TCCAGAAGAGCCACAAAAGC |
| | TCCCAAGTGCTGGGTTAAAG |
| 204O17S | TCACCTCTTATGCCCAGACC |
| | AGTCCGGTAGTTGGGAACCT |
| 326G12S | TCACCACCCAGTGAGAAAAA |
| | GGCACCATGGAAGCTAGAAG |
| 285H22T | AGCCAAGGCTCCATAGTGAA |
| | GGGCACCATTTCTGCTTCTA |
| 28H12S | TAGAAGCATCCTCCGACCTT |
| | GACATCAGGTTTGCTGGTTTT |
| 94F3S | ACCGTGTAGATAGCCGAAGC |
| | CCCACTCCAATTGTCCTTGT |
| 342J16T | AGCAGGATAACCAGGACCAT |
| | TCTGGCTGTTCTGGAACTCA |
| 350H2T | TTTGTGTCAGCTTGCCAGAC |
| | TTCCAAATTGCAGAGCTTGA |
| AA259576 | CCTCTAGATGCATACATGTGCC |
| | AGATGCATACATGTGCCCAGACA |
| 7G3S | GAGGCTGAGCAGTGAGAAAGC |
| | GGCTGGCTCAGTACTCTACCC |
| 7G3T | CCATTGATAGGCCCTTGGGCG |
| | CCCACCAGCTGCTGGCAAGGC |
| 149S | CTGTAGACTTACAGCATTTGGA |
| | GATAACTGGGCACTATGATTG |
| 149T | GCTCTAGACAACTACAATCAG |
| | GTTTCTTGGCTACTGTGTCTTC |
| 189T | GGCTGCTGTAGGCAGCTTCAGT |
| | GATAAACTTGTGAGCTGTAAGGTG |
| 80P6S | CTGTAGACCATGTCCTGAAAC |
| | CAATGTACCTCAGTATCTCCAC |
| 80P6T | GCTGCCTCCTGGAGTGGATTG |
| | GTACGTGCGTACATGTGCATG |
| 273L17S | CACCATCCCAGTCTGGTGGCA |
| | CTCAGGATGCCTCTCCATCC |
| 312B10S | CCTGCTCTGGGCAGCACAACT |
| | GCTGTCCTATGGTTGTAAATGC |
| 367M7T | GGTTACAGATGTAGTGATCAT |
| | CAGGGTGGATCGTCCCTTCTAG |
| 376F21S | GTAGACCAGACTTACCTTGAAC |
| | CATGCATGGATGATAGTTCATG |
| 475H23S | CTTGTCCTCTGGTCTCCACAGC |
| | GGTATCCTTAAGTGTCAGATC |
| 475H23T | CTAGGTGGCTCTTGGGACACTG |
| | CACCCTCTGTCATTGCCAGAC |
| 376F21T | CAGTGTGGACCCAGATGAGAAG |
| | TATGCCTGGTCAGGTTTGAAA |
| 367M7S | GAAAGGTGCCAGACCTAGCAG |
| | GCTACTAAATAGCCACATACAC |
| 435M18S | GCCTTTAGTAAGAGAGCTGAG |
| | GGAGGTGAACAGGTGTGGAG |
| 435M18T | GAAGCCTGAGAGCCACCGTTG |
| | CACTGTTGTCCACACTGTCAG |
| 354K16S | GCCGTCGACATTTAGGTGACAC |
| | CAAGGAGCCAGTGCTGATCAA |
| 354K16T | GATTATCCTACCCAGCCTTTAG |
| | GTGTAGGATATGCCATCACCAG |
| 412D2S | GTGTCAGAGTTCCTGCTTCTGG |
| | CATGTCTGGGTGCAGATGGCTC |
| 412D2T | GCTCTGAGGACTCAGGCTCCC |
| | CACTATGGCACCTGGCAGTTTC |
| 311N20S | GCCTGAGCTACCTGGTAGATCC |
| | CATTCTCATTCAAACTACTGCAG |
| 311N12T | GCCTGGGTTACATAAGACCTTG |
| | ATCCTTGAAGTCCTTAGGGTGC |
| 359B13S | GTGAGGCGAGCTTAGGGAAGAA |
| | GACGGACAGCCTCTCAAAACG |
| 359B13T | CTAGGATCCCATGGTGAAAGTAG |
| | CTACCTGGCAGTGGTAATCTG |
| 362M12S | CACCATAACTCAGAAGATCTCA |
| | CCTTCTTGAAGATTTAGTGCGC |
| 362M12T | GTTGCCAGGCCAGAACATATTC |
| | CATCATGTGAGTCACTAGCAGG |
| 405I24S | GTTGATCTTTCTAGGGACTCAG |
| | GAAGCCAAGTGGTTTTATCAG |
| 405I24T | CCAAGTACACTTGTAGCAGTC |
| | GTTAAAATTGGAAGAATAGGGC |
| 258A11S | GAATGCCTCAGGCAGAGCATCA |
| | GGACCATGTCCCTCCTGGCACT |
| 258A11T | GTGGCAAAGCAGACAGGGTAAG |
| | CAAGTGTATCTGTCCAACCAGT |
| 378A17S | CATAAGGCATACCTACTAAGTG |
| | CCAAAGCTCACTCTGCCTTGTG |
| 378A17T | GTGGAGTGAGGAACTGGCACAT |
| | CTGGCCTTCAGAGTAGCTCCCA |
| 211P17S | GACATTCTGAACAGCATACTGAT |
| | CTTGTGGCATTGTCTCCCTCATTA |
| 278O10S | CCTGCACTGACATTGGCTACACTA |
| | GTGGCAGCCATCTAGGCCAGTTCC |
| 278O10T | GAGTGTCTGAGCCTCCACTCCA |
| | CAGGGTCCAGCATGAGACCTTGGA |
| PEX11B | CACCACAGTGTCAGTCAGGGCTTG |
| | CCTCTCTTGCTGGATGTGCTCAGA |
| 98N19S | GTACTCACTGGATAAAGTGGG |
| | CTTCCATTCCTTCTTGAAGTTTCAATGAG |
| 98N19T | CATTACATCAGCTCATACACC |
| | CGAGTGTGTGCAAAGGCCCTG |
| 97A19S | CATTGTGGGCTACAGGCTTAG |
| | GAACAGCAGGAGTAGCTCCCTAC |
| 97A19T | GACTGGCTCCTTAGCACAAGG |
| | CATGATTTGTGGGTTGTGACAGC |
| 94N9S | GGCATTTCACTCACGACCCATC |
| | GTGACATGAGCTGCAAATTCTAG |
| 94N8T | CAGTCAACGCATAGGTGACTCC |
| | GCCGAGGTGGTAATATACCACC |
| 145E10S | CTCCTCCGTCGTCCCCGGTG |
| | CAGCTTCGGGATCCAAGGATG |
| 145E10T | GCCTGCCTACATGTATTGAGC |
| | CTTTAGAGACTGCTCAAGCAGC |

### EXAMPLE 11

The alignment of mouse HYPLIP1 cDNA and mouse genomic DNA using CLUSTAL X (1.8) program is shown below:

| | |
|---|---|
| TIF_cDNA | ------------------------------------------------------------ |
| TIF_genomic | TTTTTTTTTAAAAAACAGGTTTGAGGTCATCCTTGGCTATTATAGCAAGTTTGGGGCCAG |
| TIF_cDNA | ------------------------------------------------------------ |
| TIF_ganomic | CCTGGGACACATGCAACCTTGTCCAAAAAAAAAAAAAAGTCTCTTTGAATTCTTTTTTTT |
| TIF_cDNA | ------------------------------------------------------------ |
| TIF_genomic | TGGTTTTTCGAGACAGGGTTTCTCTGTATAGTAGTCTGGGTAGTCTCAAATCCCACAGAC |
| TIF_cDNA | ------------------------------------------------------------ |
| TIF_genomic | TCATTTCACAACCCCCACCCCTAAACTACCTTCTTAGGGAAAGACAGGAAAGAAGTAGGC |
| TIF_cDNA | ------------------------------------------------------------ |
| TIF_genomic | AGATGAAGGAAAAGACATATTTTACAGTGATTAAGAAACCAAGCTGTTTTGCATCCCTAG |
| TIF_cDNA | ------------------------------------------------------------ |
| TIF_genomic | CTCTGACTGTCTGCGGGGGCCAGAGGTGAGAGAATAAGGACCTGCAGGCCTGGCTTCACC |
| TIF_cDNA | ------------------------------------------------------------ |
| TIF_genomic | TCCTGTGAAGGCTGCACTGCCAGCTTTGGCACCCGGTTGTCTAGAGTAAAACAAACACAG |
| TIF_cDNA | ------------------------------------------------------------ |
| TIF_genomic | GACAAACATTCCTGGCTTCCTACTGGCGCTGAGACTGAACTTGCAAGCCTCTGCTCCCCC |
| TIF_cDNA | ------------------------------------------------------------ |
| TIF_genomic | TGGGCACAGCTGTCCTTGTCCCTGAACCCACAGCCTCTGCCCTGTTTTTGTTTATAAGAC |
| TIF_cDNA | ------------------------------------------------------------ |
| TIF_genomic | TTTTTTTTCTTCCATCCAAGAACTGAGATGGGTACGTGCGTACATGTGCATGTGCGTGTG |
| TIF_cDNA | ------------------------------------------------------------ |
| TIF_genomic | AGTGTGCGTGTGTGTGTGTGTGTGTGAGAGAGAGAGAGAGTGAAGAGGGACAAACTGCTA |
| TIF_cDNA | ------------------------------------------------------------ |
| TIF_genomic | TGAGAATACCAGGTGAAAGGTTATAAACAATCCACTCCAGGAGGCAGCCAATTCAGAACA |
| TIF_cDNA | ------------------------------------------------------------ |
| TIF_genomic | AGCCTTGGCTATAGGCCCAGGAAGCAGCTGCCACTGCCAGAGTTAAACAGATTTTTGGCC |
| TIF_cDNA | ------------------------------------------------------------ |
| TIF_genomic | TAACGCAGAACAAACAAGTGGTCTGTGCTGAGCGCCGCAAATTAAGAAACGATAGCCGTG |
| TIF_cDNA | ------------------------------------------------------------ |
| TIF_genomic | CAGGGGACAGGACACAGAACTGTCCACAGGTTTTTCCTTAATTAAAGAATTCAATACTCC |
| TIF_cDNA | ------------------------------------------------------------ |
| TIF_genomic | ATAGACAACACCGAATAACTATCAGCATTGCCTCCAAGGAGACAGCCCAAGGCAGCACCC |
| TIF_cDNA | ------------------------------------------------------------ |
| TIF_genomic | TCTCACCCCTTAGCAGCCTCCCCTCCTTCATCTGACCTCAAGGTTTAAAAACAAGAACTT |
| TIF_cDNA | ------------------------------------------------------------ |
| TIF_genomic | TTTTACATTTAAATTTTTTATTTTGGGTGTCTCTGGAGTGACTACTGGAGAGGGGAAGGA |
| TIF_cDNA | ------------------------------------------------------------ |
| TIF_genomic | GAGGGGAGGGGGAGAGGGGAGGTCAGAGTTGGTTCTTTTGCTACTGTGTGGGTTCTGCTA |
| TIF_cDNA | ------------------------------------------------------------ |
| TIF_genomic | TTGAACTCAGGTTGGCAGGCCTGGCACCATCTCTCTGGTTCTCCTGAAGTCTTATGTAGC |
| TIF_cDNA | ------------------------------------------------------------ |
| TIF_genomic | TGGGGCTGAAGAGATGGCTTGGTGGTTGATAGTATCAGAGGAAACGAGTTCGAGTCCCAG |
| TIF_cDNA | ------------------------------------------------------------ |
| TIF_genomic | AACCAAAAAACGGCAGCTCACAACTCTTAACTCCACTTCTAAGGCATCGGCGGACACCTG |
| TIF_cDNA | ------------------------------------------------------------ |
| TIF_genomic | CAGCAAGCACACAGGTGGTAGAAGAAAATAACAGCCATATACTTACAAAATTTTTAAATC |
| TIF_cDNA | ------------------------------------------------------------ |
| TIF_genomic | TTATGTTGCTACATACCACACTATTTAACAACATCGATATATGAACTTTCGGTATATTTT |
| TIF_cDNA | ------------------------------------------------------------ |
| TIF_genomic | GATATTTCATACCATCAAGCTAAGGTTTTCTCAGATGCCTGCTACAGGCACTGAGAAACT |
| TIF_cDNA | ------------------------------------------------------------ |
| TIF_genomic | GAAGTTAGTGAGCGACCTACCTCCCTTACAGTATTCATAAATACTGTTTATCGTTGGAAA |
| TIF_cDNA | ------------------------------------------------------------ |
| TIF_genomic | ACACCTGACGCCTAGTTAGTTAACTTTCTGGAACAAACACACCCTAAGGATCAAGGTGTT |
| TIF_cDNA | ------------------------------------------------------------ |
| TIF_genomic | CCTAGGCCTTGGTGTTGTGTATATGTTTTTGAACCGTGTGATGTTCATCTCTGTGCTTGC |
| TIF_cDNA | ------------------------------------------------------------ |
| TIF_genomic | TTAAGGTTCAGTTGTAACTTGTTAGCCTTAGGGTGTCAACCCAGTTAGGGCGCGCGGGGG |
| TIF_cDNA | ------------------------------------------------------------ |
| TIF_genomic | TGGGGGCTGGGGGTGTTGTTTATGAACAGCGGTGAACAGGCATGCAATCGCTTTTTACTT |
| TIF_cDNA | ------------------------------------------------------------ |
| TIF_genomic | CTCCATCTTAATCTCAGGGCTATATCATCTTTATTTTCCTGCGCAAGGAAGGAGATAGAT |
| TIF_cDNA | ------------------------------------------------------------ |
| TIF_genomic | AGTCTCCTAATAATTCTGCCCAAATATGGAAGGAGTTTAGGACTCAATGACAAGGCTCCG |
| TIF_cDNA | ------------------------------------------------------------ |
| TIF_genomic | GCGCGGGGTGGGGGTGGGGGTGGGAGTGGGTGGGTGGGTGGGGAATAGAGTAGGGGGCGA |
| TIF_cDNA | ------------------------------------------------------------ |
| TIF_genomic | AGGGGGAGGGGGTTGCAGGTAATCCTTCACACAAGAGTTTCTTTGCACACTTAAGAGTTA |
| TIF_cDNA | ------------------------------------------------------------ |
| TIF_genomic | TTTCTCTAGTCAGCTCCTGAGGCATCTCTCAGCAAGGTTTGCCAGATAACTAAGTGAAAC |
| TIF_cDNA | ------------------------------------------------------------ |
| TIF_genomic | TAACACAGCTCCAGCGCCGGTGAAATTGAAACAGGCTTAGGGACATGCATTTCATTTAGT |
| TIF_cDNA | ------------------------------------------------------------ |
| TIF_genomic | GAATTTGGAGAGAGGACAGAGGGGGGAAAAGAATGACAGGAACTCGAAAACAAAGTAAGG |
| TIF_cDNA | ------------------------------------------------------------ |
| TIF_genomic | AGTGAGGTTCTTTTTCTTCCTTTTTCTTTCTTTCTTTTATTTTATTTTTTTGGTTTGTCC |
| TIF_cDNA | ------------------------------------------------------------ |
| TIF_genomic | ACCTCTTGTTTCCTGGAGAAACAAGGACGGGGGAGCCATCAGTGTGAAAGTAAACACCTC |
| TIF_cDNA | ------------------------------------------------------------ |
| TIF_genomic | ACAAAGCTGCAGTGAGGAACAAGGGAACATATACAAAATGTTCCCCAACTTCACAGGTAC |
| TIF_cDNA | ------------------------------------------------------------ |
| TIF_genomic | ACTGAAGAGATGAGGGGATAAGCAACAGGATGTGGACACTCCCTTACTGCTTCCGCTCCA |
| TIF_cDNA | ------------------------------------------------------------ |
| TIF_genomic | GAGAACAGAATAGAATGTAATGGGCGAGGAACAGTAGCAGCACATAGGGGCATGAAATGA |
| TIF_cDNA | ------------------------------------------------------------ |
| TIF_genomic | GGGGGAAATGAGGGGAACCCACCAGAGCATTCACCAGAAAGGACTGAAAGCCAGACTTTA |
| TIF_cDNA | ------------------------------------------------------------ |
| TIF_genomic | AAATATCTGACAAGTTCTCGTCTGGAGAGACCGCAGCCTTTTATTCTTCAATAGAAGTGC |
| TIF_cDNA | ------------------------------------------------------------ |
| TIF_genomic | AATAGGAGCATATCGGGTGGGCTCTTTCTCACTAACACGACTGCACTCTCGCCCTCCGCT |
| TIF_cDNA | ------------------------------------------------------------ |
| TIF_genomic | CCATCCTGGAGTATCCTCGGTGCGATGGGATTGTTTTTCACAAGACTTGCGAACTTGTGA |
| TIF_cDNA | ------------------------------------------------------------ |
| TIF_genomic | GCCAGGAATAAATGGTCACCTCGAAATGAATTGCGCTGGCTCAGGCGAGTCATGAAATCC |
| TIF_cDNA | ------------------------------------------------------------ |
| TIF_genomic | TCTCCTAAGCACATTTTTCTTTCACCTAAAAAAAGAAGGGGGAAAAAAAAAACAAAGCAC |
| TIF_cDNA | ------------------------------------------------------------ |
| TIF_genomic | ACACCCAAATAACCCAGCTCCCAAGAGGAGTCCCCTGGATGAGGTTCAGGGTCCCGGGGT |
| TIF_cDNA | ------------------------------------------------------------ |
| TIF_genomic | CCCAGCCTCCCGGGGGGAGGGAGGGCACCCGTCGCCCCGGGCCCCGCCCCTCCTGCTGGC |
| TIF_cDNA | ------------------------------------------------------------ |
| TIF_genomic | AAGGCTGCGCACCCGAACAACAACCATTTTCCCCGCTAGGAGCACACCGTGTCCACGCGC |
| TIF_cDNA | ------------------------------------------------------------ |
| TIF_genomic | CCCGGCGGCCTCGCTGATTGGTTGGAGGCCTGGTAAACAAGGGCCAAGTAGCCAATGGGA |
| TIF_cDNA | ------------------------------------------------------------ |
| TIF_genomic | GAACTGTGCACGAGGGCTGCACGAGCCTCCAGGCCAGCACTCGCGTGGAGCGCCAAGCCA |
| TIF_cDNA | ------------------------------------------------------------ |
| TIF_genomic | GGCGGCTATATAAGCCGTNTCCGGCAGCCGGTTGACACTCTTCCTCCTCTGGTCTCGGGG |
| TIF_cDNA | ------------------------------------------------------------ |
| TIF_genomic | TTTCCAGAGTTTCTCCAGTTGCGGAAGACAGCTGTTATTTTTCTCCTGAAAGCTTTTGGC |
| TIF_cDNA | ------------------------------------------------------------ |
| TIF_genomic | ACAGCCGGCAGGCTGAAACTTCCAGGCACCTTTTGGAAAAGTTGTTAGGGTTTGTTTGAA |
| TIF_cDNA | ------------------------------------------------------------ |
| TIF_genomic | GCTTTCTTTACATTTTCGTTTGGGTTTTCAAGCCCTGACTTTACGGAGGCGAGCTCTTCG |
| TIF_cDNA | ---------------------------TTTTCCTCTCCGGCTTTCGTTTTTCTTGAACCC |
| TIF_genomic | TTTGCTTTGAAGGGTTCTTAAAGATTTTTTTCCTCTCCGGCTTTCGTTTTTCTTGAACCC |
| | ********************************* |
| TIF_cDNA | ACTCGGCTCAATCATGGTGATGTTCAAGAAGATCAAGTCTTTTGAGGTGGTCTTCAACGA |
| TIF_genomic | ACTCGGCTCAATCATGGTGATGTTCAAGAAGATCAAGTCTTTTGAGGTGGTCTTCAACGA |
| | ************************************************************ |
| TIF_cDNA | CCCCGAGAAGGTGTACGGCAGCGGGGAGAAGGTGGCCGGACGGGTAATAGTGGAAGTGTG |
| TIF_genomic | CCCCGAGAAGGTGTACGGCAGCGGGGAGAAGGTGGCCGGACGGGTAATAGTGGAAGTGTG |
| | ************************************************************ |
| TIF_cDNA | TGAAGTTACCCGAGTCAAAGCCGTCAGGATCCTGGCTTGCGGCGTGGCCAAGGTCCTGTG |
| TIF_genomic | TGAAGTTACCCGAGTCAAAGCCGTCAGGATCCTGGCTTGCGGCGTGGCCAAGGTCCTGTG |
| | ************************************************************ |
| TIF_cDNA | GATGCAAGGGTCTCAGCAGTGCAAACAGACTTTGGACTACTTGCGCTATGAAGACACACT |
| TIF_genomic | GATGCAAGGGTCTCAGCAGTGCAAACAGACTTTGGACTACTTGCGCTATGAAGACACACT |
| | ************************************************************ |
| TIF_cDNA | TCTCCTAGAAGAGCAGCCTACAGGT----------------------------------- |
| TIF_genomic | TCTCCTAGAAGAGCAGCCTACAGGTACTGCTCCCAGCAGGACTGATGGTGACTTGGGAGG |
| | ************************* |
| TIF_cDNA | ------------------------------------------------------------ |
| TIF_genomic | TCTGTGGGTCGGGGAGGGCACCACTAAATGTTTCGAGTTGTTCGTTTGAATGGTTTGAAC |
| TIF_cDNA | ------------------------------------------------------------ |
| TIF_genomic | TGTTGGTCCCTATATTTTTTTACTTTGTAATTAGCAAGTTTTTCACTACCCTTCACCCCC |
| TIF_cDNA | ------------------------------------------------------------ |
| TIF_genomic | CTAGAGTGATTTGAACACTTTCTGAGGTACTGTTTCCTGAAAGTGTTGTCTTAGCTACTA |
| TIF_cDNA | ------------------------------------------------------------ |
| TIF_genomic | CTTAAAGATTAATGTATTTGTGGATTTCGCAACTTTCTGTCCAAGAAAGTGCTCTGGGAT |
| TIF_cDNA | ------------------------------------------------------------ |
| TIF_genomic | CTTTTCTTCCATAGTGTAAGAGATGAAAGTGGAAGTGAAGTAAGGTAGTCTACTGCCCAG |
| TIF_cDNA | ------------------------------------------------------------ |
| TIF_genomic | GCACTCCTCATTGACGCTTTCAAAATGTAACAAGAAGCCTAATGGCCCCTTGTCTTTGTT |
| TIF_cDNA | -----------GAGAACGAGATGGTGATCATGAGGCCTGGAAACAAATATGAGTACAAGT |
| TIF_genomic | TCCCAGCAGGTGAGAACGAGATGGTGATCATGAGGCCTGGAAACAAATATGAGTACAAGT |
| | ************************************************* |
| TIF_cDNA | TCGGCTTCGAGCTTCCTCAAGGG------------------------------------- |
| TIF_genomic | TCGGCTTCGAGCTTCCTCAAGGGTAGGCATCCACCGTGTGCACCTTGCACTCTTATTTCT |
| | *********************** |
| TIF_cDNA | ------------------------------------------------------------ |
| TIF_genomic | AAGTCTTCCCCCTCCATTGATCTCTTACAGTTCTTAGCCTTAATTTTGGTTCATTGTTTT |
| TIF_cDNA | --------CCCCTGGGAACATCCTTTAAAGGAAAATATGGTTGCGTAGACTACTGGGTGA |
| TIF_genomic | GACACAGGCCCCTGGGAACATCCTTTAAAGGAAAATATGGTTGCGTAGACTACTGGGTGA |
| | **************************************************** |
| TIF_cDNA | AGGCTTTTCTCGATCGCCCCAGCCAGCCAACTCAAGAGGCAAAGAAAAACTTCGAAGTGA |
| TIF_genomic | AGGCTTTTCTCGATCGCCCCAGCCAGCCAACTCAAGAGGCAAAGAAAAACTTCGAAGTGA |
| | ************************************************************ |
| TIF_cDNA | TGGATCTAGTGGATGTCAATACCCCTGACTTAATGG------------------------ |
| TIF_genomic | TGGATCTAGTGGATGTCAATACCCCTGACCTAATGGTGAGGATTTTTTGTTTTTGTTTTT |
| | ***************************** ****** |
| TIF_cDNA | ------------------------------------------------------------ |
| TIF_genomic | AAAAAGGTTTTAAAATTCTTCTTGGTCAGGGATAATAAATTAGATGCATGGGGGTTGAAA |
| TIF_cDNA | -------------------------------CACCAGTGTCTGCCAAAGAGGAGAAGAAA |
| TIF_genomic | TATCTCAAAACATTATTTCCTTTTACACAGGCACCAGTGTCTGCCAAAAAGGAGAAGAAA |
| | ***************** *********** |
| TIF_cDNA | GTTTCCTGCATGTTCATTCGTGATGGACGTGTGTCAGTCTCTGCTCGAATTGACAGAAAA |
| TIF_genomic | GTTTCCTGCATGTTCATTCCTGATGGACGTGTGTCAGTCTCTGCTCGAATTGACAGAAAA |
| | ******************* **************************************** |
| TIF_cDNA | GGATTCTGTGAAGGT--------------------------------------------- |
| TIF_genomic | GGATTCTGTGAAGGTAAAAACATACTGCTTCAAATGCTAGACAGGATAGCCAGAACTGGG |
| | *************** |
| TIF_cDNA | ------------------------------------------------------------ |
| TIF_genomic | GGTGGGGGGGTTGGGGGTGGTACGGAGAGGGTCGTAGGGTAGAGGCAGAGGAAGTGCTGT |
| TIF_cDNA | ------------------------------------------------GATGACATCTCC |
| TIF_genomic | TAACTTGCATGGCTATTCATACTTCCTCATTTTATTTTAACTCTAGGTGATGACATCTCC |
| | ************ |
| TIF_cDNA | ATCCATGCTGACTTTGAGAACACGTGTTCCCGAATCGTGGTCCCCAAAGCGGCTATTGTG |
| TIF_genomic | ATCCATGCTGACTTTGAGAACACGTGTTCCCGAATCGTGGTCCCCAAAGCGGCTATTGTG |
| | ************************************************************ |
| TIF_cDNA | GCCCGACACACTTACCTTGCCAATGGCCAGACCAAAGTGTTCACTCAGAAGCTGTCCTCA |
| TIF_genomic | GCCCGACACACTTACCTTGCCAATGGCCAGACCAAAGTGTTCACTCAGAAGCTGTCCTCA |
| | ************************************************************ |
| TIF_cDNA | GTCAGAGGCAATCACATTATCTCAGGGACTTGCGCATCGTGGCGTGGCAAGAGCCTCAGA |
| TIF_genomic | GTCAGAGGCAATCACATTATCTCAGGGACTTGCGCATCGTGGCGTGGCAAGAGCCTCAGA |
| | ************************************************************ |
| TIF_cDNA | GTGCAGAAGATCAGACCATCCATCCTGGGCTGCAACATCCTCAAAGTCGAATACTCCTTG |
| TIF_genomic | GTGCAGAAGATCAGACCATCCATCCTGGGCTGCAACATCCTCAAAGTCGAATACTCCTTG |
| | ************************************************************ |
| TIF_cDNA | CTG--------------------------------------------------------- |
| TIF_genomic | CTGGTGAGTGGGTGAGAAGAGAGACAATTACCTGGTTACAAATTCAGTGCTTTCTGTACT |
| | *** |
| TIF_cDNA | -----------------------------------ATCTACGTCAGTGTCCCTGGCTCCA |
| TIF_genomic | CAACCCATCTAACAAACTGCCATCCTCCTCTCTAGATCTACGTCAGTGTCCCTGGCTCCA |
| | ************************* |
| TIF_cDNA | AGAAAGTCATCCTTGATCTGCCCCTAGTGATTGGCAGCAGGTCTGGTCTGAGCAGCCGGA |
| TIF_genomic | AGAAAGTCATCCTTGATCTGCCCCTAGTGATTGGCAGCAGGTCTGGTCTGAGCAGCCGGA |
| | ************************************************************ |
| TIF_cDNA | CATCCAGCATGGCCAGCCGGACGAGCTCTGAGATGAGCTGGATAGACCTAAACATCCCAG |
| TIF_genomic | CATCCAGCATGGCCAGCCGGACGAGCTCTGAGATGAGCTGGATAGACCTAAACATCCCAG |
| | ************************************************************ |
| TIF_cDNA | ATACCCCAGAAG------------------------------------------------ |
| TIF_genomic | ATACCCCAGAAGGTAAGCTGCAGCCGGATAGGTTCGAGTTATTTTGATCTGCTTGGGCTT |
| | ************ |
| TIF_cDNA | ------------------------------------------------------------ |
| TIF_genomic | GTGGAGTTGGGGTGACCTGGCATTTATTTCTTAGTCGGACTTCTGACACCGTTTTCTCTC |
| TIF_cDNA | -----CTCCTCCTTGCTATATGGACATCATTCCTGAAGATCACAGACTAGAGAGCCCCAC |
| TIF_genomic | TTCAGCTCCTCCTTGCTATATGGACATCATTCCTGAAGATCACAGACTAGAGAGCCCCAC |
| | ******************************************************* |
| TIF_cDNA | CACCCCTCTGCTGGATGATGTGGACGACTCTCAAGACAGCCCTATCTTTATGTACGCCCC |
| TIF_genomic | CACCCCTCTGCTGGACGATGTGGACGACTCTCAAGACAGCCCTATCTTTATGTACGCCCC |
| | *************** ******************************************** |
| TIF_cDNA | TGAGTTCCAGTTCATGCCCCCACCCACTTACACTGAGGTG-------------------- |
| TIF_genomic | TGAGTTCCAGTTCATGCCCCCACCCACTTACACTGAGGTGAGAACTGCTATTCTCACAGG |
| | **************************************** |
| TIF_cDNA | ------------------------------------------------------------ |
| TIF_genomic | GTCAACATTTTGTCCTAGGCCTTTTGAAGGAAGGGTTAATGTGGGTTTTCTACTTAACTA |
| TIF_cDNA | ---------------------------------------GATCCGTGCGTCCTTAACAAC |
| TIF_genomic | AAAAACCTGAAAATTTCCTCTCTATTCCCCTTCCAGGTGGATCCGTGCGTCCTTAACAAC |
| | ********************* |
| TIF_cDNA | AACAACAACAACAAC---GTGCAGTGAAGCTGCAGGAAATGAAGCATCTGTAT-AGCGCA |
| TIF_genomic | AACAACAACAACAACAACGTGCAGTGAGCCTGCAGGAAATGAAGCATCTGTATTAGCGCA |
| | *************** ********* ************************ ****** |
| TIF_cDNA | TT-CTTTCTGCCTCTCTGCTTGAACTC-AGTGTTTCAGAGACTCAGTCTCTACAGCGGGG |
| TIF_genomic | TTTCTTTCTGCCTCTCTGCTTGAACTCCAGTGTTTCAGAGACTCAGTCTCTACAGCGGGG |
| | ** ************************ ******************************** |
| TIF_cDNA | AACGGGTACACCCCAGCCGCTGACTCC-CAAGATGGGTGGCAATCAGTAGGCGGGTCTCC |
| TIF_genomic | AACGGGTACACCCCAGCCGCTGACTCCTCAAGATGGGTGGCAATCAGTAGGCGGGTCTCC |
| | *************************** ******************************** |
| TIF_cDNA | GGCTTCAAGTGGTGCAGACCAGTGCCC-CACTGTGGCATAGGAGTGTTTGCTGGGTGGAT |
| TIF_genomic | GGCTTCAAGTGGTGCAGACCAGTGCCCGCACTGTGGCATAGGAGTGTTTGCTGGGTGGAT |
| | *************************** ******************************** |
| TIF_cDNA | GTCAGAACACTCTT---------------------------------------------- |
| TIF_genomic | GTCAGAACACTCTTAGAAAAATTGAGACCTGACCACTTTCTCGGATGTTGGAAATGAAGA |
| | ************** |
| TIF_cDNA | ------------------------------------------------------------ |
| TIF_genomic | ACTTGTTTGTGTTGACTGAGTCAGGGCACTGCTGACCTTCTGGCGTTGTCTTTCCAAGGT |
| TIF_cDNA | ------------------------------------------------------------ |
| TIF_genomic | TTTTGTTTTAAAGGGACTTTTAAATTGTCTAAAATATCAGTAGACCATCATCTGTGCCAT |
| TIF_cDNA | ------------------------------------------------------------ |
| TIF_genomic | GGGGGACAGAGCCAATTTCAAGTCATGGCCAAAATTTTGTAAGAGGAGTGTTTTTGTGTG |
| TIF_cDNA | ------------------------------------------------------------ |
| TIF_genomic | TTTTTTAAAGTCAGTGTTCCTTTTTTATATCTTTACAAAGAAAAGACCTTCCACGGCTGG |
| TIF_cDNA | ------------------------------------------------------------ |
| TIF_genomic | TGAGCACGCAGCCTGTGAAATTCGGGGCAGCTGCTCCAAGTTGACTTCACCCTGGGAGCA |
| TIF_cDNA | ------------------------------------------------------------ |
| TIF_genomic | GTAGTAGCTGTGCCCACTGACGGCCATAAAAGCCATTTTACAGCCAGTTGCACTGTGTTC |
| TIF_cDNA | ------------------------------------------------------------ |
| TIF_genomic | TCTTGTAAGCATAATCAGATGGGAGAATCTGTTATTTCCCTGTAACCCCTTGGAATTGAT |
| TIF_cDNA | ------------------------------------------------------------ |
| TIF_genomic | TCTAAGGTGATGTTCTTAGCACTTTAGCTTGTCAATTTTGTTTTAGTCTCCGTTATAGAT |
| TIF_cDNA | ------------------------------------------------------------ |
| TIF_genomic | GTAAGCTCCACCAGTCTCTTAAGGATTAAGCCCAGTGACTTGGAGGGTGGGGGTTAGGGT |
| TIF_cDNA | ------------------------------------------------------------ |
| TIF_genomic | CTCTATCCCTGAACATTGTAGACCCAGGCTGGCCTGAGAGATCCACCTGCCTCTGCCTCC |
| TIF_cDNA | ------------------------------------------------------------ |
| TIF_genomic | TGAGTGCTGCGATCAAAGGCCCAGCTTGGTTATTGCTTTTGAGGCTTTCTCCCAACGCAC |
| TIF_cDNA | ------------------------------------------------------------ |
| TIF_genomic | AGACTTGTGTAATTCTAACACTAATCCTGTGAAGGGTTGTGGTTGACAGCTGGAGCCTGG |
| TIF_cDNA | ------------------------------------------------------------ |
| TIF_genomic | GTGACATTCTACATTGAGATGCCCCAGCACTGATCGGGGCACAGAAGCCCCCAGACCCCA |
| TIF_cDNA | ------------------------------------------------------------ |
| TIF_genomic | TTTCCTGTCCAGTGTTGGGAGAAAGTGCTGCTTTCACTGTGGCCTCAGCCCTGGCTCGGA |
| TIF_cDNA | ------------------------------------------------------------ |
| TIF_genomic | AGCTCACTAAGCCTTAGCACTTTGTCCTGTGTCAGCTCCACCTGAGAACTGTGCAGCCAG |
| TIF_cDNA | ------------------------------------------------------------ |
| TIF_genomic | AATGTCTGCGAGCTGATGGAGGTTTCGGTTTTGTTGTTTTTGTATTTTGTGTATCTTTTT |
| TIF_cDNA | ------------------------------------------------------------ |
| TIF_genomic | GTATGATTAAAAACTATATTTTCTACTTATCCAAATATATTTTCACCCCAAAGTGGGGTT |
| TIF_cDNA | ------------------------------------------------------------ |
| TIF_genomic | ATCCTTTGTAAAAAAAAATAAAGTTTTTTAATGACAAAAATAAATGTTCTTTTCTTGTCT |
| TIF_cDNA | ------------------------------------------------------------ |
| TIF_genomic | ATGAGATACTGGAGAAGTTACTAGAAAGTGTTCCCCTGTCTCAATACTGAAAGCCCGTGG |
| TIF_cDNA | ------------------------------------------------------------ |
| TIF_genomic | AGAGAGAAGTCTCTTGACGCTGAGTGACATAACGGCTGGTTTGGCCTCTGTTCAGACGGA |
| TIF_cDNA | ------------------------------------------------------------ |
| TIF_genomic | GGAATCCGTAGGGTCTGGTAGTAGAAGCTAATTAACCACGTCCATAGTCAGAAAACTCCT |
| TIF_cDNA | ------------------------------------------------------------ |
| TIF_genomic | TCAGGATCAGGCTTGCTCCTGGGACTGAGGATAGCCTTGAACCTCTGGTGCAGCCATCAA |
| TIF_cDNA | ------------------------------------------------------------ |
| TIF_genomic | GAGCACGCAGTGTCATGCTCAGGTTTTCATAGTTTGTGTGTGTGAATGCAGGTGGGAATG |
| TIF_cDNA | ------------------------------------------------------------ |
| TIF_genomic | TGGTGCTTAGAACCCACCTTGCAAAAGTCAGCTCCACTTTGTGGGACCCTGAGACCAGGA |
| TIF_cDNA | ------------------------------------------------------------ |
| TIF_genomic | CCTCAGGCTTCGCAGAAAGCGTCTTTTACTGCTGAGCCATCTCTGAGCCCAGTTCTCTGC |
| TIF_cDNA | ------------------------------------------------------------ |
| TIF_genomic | CCTGTTTATGAATTCTTTAAAAATAACTAAGGGGATTTGGAAGGGACAGGGTGAGATTTT |
| TIF_cDNA | ------------------------------------------------------------ |
| TIF_genomic | TATTTTTGTTAAATCCAAATGAGCAGCTTTTGTTTACACAAACGCAGGGAGGATGTGGGG |
| TIF_cDNA | ------------------------------------------------------------ |
| TIF_genomic | AAAAGGGACTGGGAGATTAATGTGAGGGAAATTAAATGGGTGTTTGCTCAGATGGGAGGC |
| TIF_cDNA | ------------------------------------------------------------ |
| TIF_genomic | AGGAAGCAGTCCTGGTGTGCTCCGGTGGATCTGATGTTCCCTAAAGCTCAGCAGACAGTC |
| TIF_cDNA | ------------------------------------------------------------ |
| TIF_genomic | CAGAGTGAGAATGGGTTCTGACTGGCAGAGGCCTCAGCCCACCCTACCCCAAAACAGGAT |
| TIF_cDNA | ------------------------------------------------------------ |
| TIF_genomic | GACTGGTGGCAATGGAGTTTTTGGTTTGGTTTGAGACAAGTTCAGGCTAGCCTTAACCTG |
| TIF_cDNA | ------------------------------------------------------------ |
| TIF_genomic | GAAGCAATCTGGCTCAGCCTCCCGAGCACTGGGGTTAGAAGACCACGGTCTCATTCATCA |
| TIF_cDNA | ------------------------------------------------------------ |
| TIF_genomic | CTTGGTTTTTATTGAGAATTCCCCCAATATAAACTTGGTTTATAAGCTGCAAAGAGGAAC |
| TIF_cDNA | ------------------------------------------------------------ |
| TIF_genomic | TATTTCAGACTTGGTTTTAGTTACAGGGATTAAATGTTTTAGAAGCAGCTACAGTTTTCT |
| TIF_cDNA | ------------------------------------------------------------ |
| TIF_genomic | GTCTTTATAGATTATTGTGTTTTTTGAGACAGGGTTTCTCTGTAGTCCTGCTCTGTAGAT |
| TIF_cDNA | ------------------------------------------------------------ |
| TIF_genomic | CAGGCTAACCCTAAACTCAGAGATCCACTTTCCTCTGTCCCCCGAATGCTGGGATTAGCG |
| TIF_cDNA | ------------------------------------------------------------ |
| TIF_genomic | TTTACCACCACAGCCTGACTCTTTACAGTTCTCAACGTATAATTAGAATTCAGTGTCTAC |
| TIF_cDNA | ------------------------------------------------------------ |
| TIF_genomic | CCTGATTCCTTGGGACCTGTTTTGGAATTTTCTATTTCTTAGAAGGGTATTGATGACTGA |
| TIF_cDNA | ------------------------------------------------------------ |
| TIF_genomic | TAAACCATTTCACTGCTAACTGAAGTTATTTTGTTCAGGAAAAAGCTACACACATGAGAA |
| TIF_cDNA | ------------------------------------------------------------ |
| TIF_genomic | ACAAAGATGGCAGAATACATCACACCATTCTTTCTGGTTTTTGGTTCATCTAAATGTTTT |
| TIF_cDNA | ------------------------------------------------------------ |
| TIF_genomic | TCGTCAAAATGGGTTTTCCATAGCTCTCCACACACCAGTACACTCTCTGAAGCACTGTAT |
| TIF_cDNA | ------------------------------------------------------------ |
| TIF_genomic | TAGAAACCAAGGGGAGGCTCGCTGTGGTCATGCACACCTAANNNNNNNNNNNNNNNNNNN |
| TIF_cDNA | ------------------------------------------------------------ |
| TIF_genomic | NNNNNNNNNNNNNNNNNNNNNNNNNNNNNNNNNNNNNNNNNNNNNNNNNNNNNNNNNNNN |
| TIF_cDNA | ------------------------------------------------------------ |
| TIF_genomic | NNNNNNNNNNNCTGTGAACACAGAACTGAACAGAAATTGAAAAAAAAAGAAATCCTTTTC |
| TIF_cDNA | ------------------------------------------------------------ |
| TIF_genomic | TGCGCTAGTAATTGATCTTTATCATTCATTCGCTATAGCGCACCTGTCACTTTCCTGCCT |
| TIF_cDNA | ------------------------------------------------------------ |
| TIF_genomic | CACTGGCGCACGCCTTTAATCCCAGCACTCGGGAGGCAGAGGCAGGCAGATTTCTAAGGT |
| TIF_cDNA | ------------------------------------------------------------ |
| TIF_genomic | CAAGGCCAGCCTGGTCTACAAAGTGAGTTCCAGGACAGCCAGGGCTACACAGAGAAACCC |
| TIF_cDNA | ------------------------------------------------------------ |
| TIF_genomic | TGTCTCAAAAAAACAAAAACAAACAAACAAAAAATAAAATAAACAATAAAACATAAATAA |
| TIF_cDNA | ------------------------------------------------------------ |
| TIF_genomic | ATAAAAAGAACAATCATTTGTGTCTGTATACCACAGTGCCCAGGAGGTCAGAGGACTTCT |

The amino acid sequence alignment among human, mouse, and rat sequences is shown below (rat amino acid sequence is assigned SEQ ID NO. 407):

CLUSTAL W (1.81) Multiple Sequence Alignments

Sequence format is Pearson

| | |
|---|---|
| Sequence 1: s73591_human.pep | 391 aa |
| Sequence 2: u30789_rat.pep | 1137 aa |
| Sequence 3: af173681_mouse.pep | 395 aa |

Start of Pairwise alignments
Aligning . . .
Sequences (1:2) Aligned.Score: 96
Sequences (2:3) Aligned.Score: 97
Sequences (1:3) Aligned.Score: 95

| | |
|---|---|
| Guide tree | file created: |

Start of Multiple Alignment
There are 2 groups
Aligning . . .

| | |
|---|---|
| Group 1: Sequences: 2 | Score:8532 |
| Group 2: Sequences: 3 | Score:8392 |

Alignment Score 7121
CLUSTAL-Alignment file created

CLUSTAL W (1.81) Multiple Sequence Alignment

| | | |
|---|---|---|
| u30789_rat.pep | FFQCRVRIHNIVFLMEVSVLPRILHDRDCPRVLWLSCNLKELALQLIKWLSVFYDSVQVI | 60 |
| af173681_mouse.pep | ------------------------------------------------------------ | |
| s73591_human.pep | ------------------------------------------------------------ | |
| u30789_rat.pep | NEFPMVILYERTKPAVLPQSVCSLPWSHRHRLCSPLFLLLPTAMNQTLVDAWSWGSKNCL | 120 |
| af173681_mouse.pep | ------------------------------------------------------------ | |
| s73591_human.pep | ------------------------------------------------------------ | |
| u30789_rat.pep | VSSVLLLHQNVMITRLPSGIQQRGMPRFKYPSIPFFATYQHFFALLAFYLFFDSTETVKC | 180 |
| af173681_mouse.pep | ------------------------------------------------------------ | |
| s73591_human.pep | ------------------------------------------------------------ | |
| u30789_rat.pep | CIQATCLPICEHMFHVMKMIRSGKEARFLNSAQIFPHWHLLSIRNPASSGLWISRVPPVA | 240 |
| af173681_mouse.pep | ------------------------------------------------------------ | |
| s73591_human.pep | ------------------------------------------------------------ | |
| u30789_rat.pep | RKQLLFFSKLLAQPAGNFQAPFREVVKVLFEAFFGFLSPLCLTERLKFLVCEGFRVFPLR | 300 |
| af173681_mouse.pep | ------------------------------------------------------------ | |
| s73591_human.pep | ------------------------------------------------------------ | |
| u30789_rat.pep | LPFFLNPLGSIMVMFKKIKSFEVVFNDPEKVYGSGEKVAGRVTVEVCEVTRVKAVRILAC | 360 |
| af173681_mouse.pep | -----------MVMFKKIKSFEVVFNDPEKVYGSGEKVAGRVIVEVCEVTRVKAVRILAC | 49 |
| s73591_human.pep | -----------MVMFKKIKSFEVVFNDPEKVYGSGERVAGRVIVEVCEVTRVKAVRILAC | 49 |
| | *************************:***** ***************** | |
| u30789_rat.pep | GVAKVLWMQGSQQCKQTLDYLRYEDTLLLEDQPTGENEMVIMRPGNKYEYKFGFELPQGP | 420 |
| af173681_mouse.pep | GVAKVLWMQGSQQCKQTLDYLRYEDTLLLEEQPTGENEMVIMRPGNKYEYKFGFELPQGP | 109 |
| s73591_human.pep | GVAKVLWMQGSQQCKQTSEYLRYEDTLLLEDQPTGENEMVIMRPGNKYEYKFGFELPQGP | 109 |
| | ***************** :***********:***************************** | |
| u30789_rat.pep | LGTSFKGKYGCVDYWVKAFLDRPSQPTQEAKKNFEVMDLVDVNTPDLMAPVSAKKEKKVS | 480 |
| af173681_mouse.pep | LGTSFKGKYGCVDYWVKAFLDRPSQPTQEAKKNFEVMDLVDVNTPDLMAPVSAKEEKKVS | 169 |
| s73591_human.pep | LGTSFKGKYGCVDYWVKAFLDRPSQPTQETKKNFEVVDLVDVNTPDLMAPVSAKKEKKVS | 169 |
| | *****************************:******:*****************:***** | |
| u30789_rat.pep | CMFIPDGRVSVSARIDRKGFCEGDDISIHADFENTCSRIVVPKAAIVARHTYLANGQTKV | 540 |
| af173681_mouse.pep | CMFIRDGRVSVSARIDRKGFCEGDDISIHADFENTCSRIVVPKAAIVARHTYLANGQTKV | 229 |
| s73591_human.pep | CMFIPDGRVSVSARIDRKGFCEGDEISIHADFENTCSRIVVPKAAIVARHTYLANGQTKV | 229 |
| | **** *******************:*********************************** | |
| u30789_rat.pep | LTQKLSSVRGNHIISGTCASWRGKSLRVQKIRPSILGCNILRVEYSLLIYVSVPGSKKVI | 600 |
| af173681_mouse.pep | FTQKLSSVRGNHIISGTCASWRGKSLRVQKIRPSILGCNILKVEYSLLIYVSVPGSKKVI | 289 |
| s73591_human.pep | LTQKLSSVRGNHIISGTCASWRGKSLRVQKIRPSILGCNILRVEYSLLIYVSVPGSKKVI | 289 |
| | :****************************************:****************** | |
| u30789_rat.pep | LDLPLVIGSRSGLSSRTSSMASRTSSEMSWIDLNIPDTPEAPPCYMDVIPEDHRLESPTT | 660 |
| af173681_mouse.pep | LDLPLVIGSRSGLSSRTSSMASRTSSEMSWIDLNIPDTPEAPPCYMDIIPEDHRLESPTT | 349 |
| s73591_human.pep | LDLPLVIGSRSGLSSRTSSMASRTSSEMSWVDLNIPDTPEAPPCYMDVIPEDHRLESPTT | 349 |
| | ******************************:****************:************ | |
| u30789_rat.pep | PLLDDVDDSQDSPIFMYAPEFQFMPPPTYTEVDPCVLNNNNNNVQAFRDDSSVLLNASAS | 720 |
| af173681_mouse.pep | PLLDDVDDSQDSPIFMYAPEFQFMPPPTYTEVDPCVLNNNNNN----------------- | 392 |
| s73591_human.pep | PLLDDMDGSQDSPIFMYAPEFKFMPPPTYTEVDPCILNN--------------------- | 388 |
| | *****:*.*************:*************:*** | |
| u30789_rat.pep | LLELNVQRLSLSSGVVHPLLHRWVAISRQVSSLKGCRPVPAPYRSVCWVNVGTLKTFRPG | 780 |
| af173681_mouse.pep | ----NVQ----------------------------------------------------- | 395 |
| s73591_human.pep | ----NVQ----------------------------------------------------- | 391 |
| | *** | |
| u30789_rat.pep | PLSQMLEMKKLLFFVGVVTFRHGLSKVFCFEGNKNRPTSVPWGNRANVTYKREIMANILK | 840 |
| af173681_mouse.pep | ------------------------------------------------------------ | |
| s73591_human.pep | ------------------------------------------------------------ | |
| u30789_rat.pep | GEECFVFNQCSLLYLYKEKRPSTAGDRAACEVRAAAPSLTVGAVVAAHTSSQVHCVLLQS | 900 |
| af173681_mouse.pep | ------------------------------------------------------------ | |
| s73591_human.pep | ------------------------------------------------------------ | |
| u30789_rat.pep | DGRTRYFRVTPNFGDVLGTLACQFLVWVIDGGSGLSPVTGKRSYYFGRVGVGPLSQNSID | 960 |
| af173681_mouse.pep | ------------------------------------------------------------ | |
| s73591_human.pep | ------------------------------------------------------------ | |
| u30789_rat.pep | QAGLKDPPASPSLLRLKAQLGYYFWRLSPNTQTSVILTLLLRVAADSWTLVTLLHDALTL | 1020 |
| af173681_mouse.pep | ------------------------------------------------------------ | |
| s73591_human.pep | ------------------------------------------------------------ | |
| u30789_rat.pep | MEAQELQVSMEAPRPISCPVLGESSVSPWPLALQNLLRLSTLFRVLCQSSKLHSKHSTRE | 1080 |
| af173681_mouse.pep | ------------------------------------------------------------ | |
| s73591_human.pep | ------------------------------------------------------------ | |
| u30789_rat.pep | LCSQKVADGGVVFVVFVFCVSFCMIKNYILYLTKYIFTQSGAILCKKKKIKIKFFNG | 1137 |
| af173681_mouse.pep | --------------------------------------------------------- | |
| s73591_human.pep | --------------------------------------------------------- | |

The rat mRNA sequence is provided in Young et al., J. Mol Carcinog 15(4), 251-260 (1996).

### EXAMPLE 12

Increased Incidence of Hepatic Tumors in HcB-19 Mice

In addition to the hyperlipidemia phenotype in HcB-19 mutant mice, an increased incidence of hepatic tumors was observed as compared to C3H controls. The ages, sex, and number of HcB-19 and C3H mice examined are listed in Table 3. The increase in tumor formation in HcB-19 mutant mice was significant (p value<0.0001). Hepatic tumors were observed in HcB-19 mice as young as 8 months of age. Besides hepatic tumors, no other macroscopic abnormalities were observed in either strain.

The majority of the tumors observed exhibited vascular invasion and angiogenesis. In addition, several animals showed evidence of metastasis, since more than one tumor was present. Further pathologic analysis of tumors from HcB-19 animals revealed the presence of both hepatic adenoma and hepatocellular carcinoma.

Segregation of Hepatic Tumors with the Nonsense Mutation in Vdup1

In order to determine if the increased occurrence of hepatic tumor formation resulted from the spontaneous Vdup1 nonsense mutation present in HcB-19 mice, we analyzed 130 animals that were derived from a backcross of (HcB-19×CAST/Ei)F2 animals to the HcB-19 parental strain. Thus, all animals utilized were either heterozygous or homozygous for the HYPLIP1 null mutation in the Vdup1 gene. The incidence of hepatic tumors in the backcross animals was significantly higher in animals homozygous for the Vdup1 null mutation (p value<0.006). The data for the ages, sex, and number of animals with or without liver tumors for each genotype are presented in Table 4.

Although evidence suggests that the hepatic tumor occurrence segregate with the HYPLIP1 nonsense mutation in the Vdup1 gene, two out of 36 animals that were heterozygous for the nonsense mutation also exhibited liver tumors. These may result from the loss of heterozygosity at the Vdup1 locus, or perhaps from additional somatic changes. A (HcB-19×C57BL/6J)N5 congenic mouse strain was constructed where it contains 97% of the C57BL/6J genetic background, and is either homozygous or wild type for the Vdup1 nonsense mutation. These animals are a resource for further investigation of the role of Vdup1 in hepatic carcinoma.

There are several lines of evidence that indicates Vdup1 may be a tumor suppressor gene. First, murine Vdup1 expression is decreased in rat mammary tumors, and up-regulation of mVdup1 by 1α,25-dihydroxyvitamin D₃ treatment inhibited tumor cell growth (Yang et al., Breast Cancer Res. Treat. 48:33-44 (1998)). Besides mammary tumors, 1α,25-dihydroxyvitamin D₃ treatment also restricts growth in a variety of cancer cell lines and primary tumors, including murine hepatic tumors, human hepatocellular carcinoma, and the human HepG2 hepatoblastoma cell line (Tanaka et al., Biochem. Pharmacol. 38:449-453 (1989); Miyaguchi et al., Hepatogastroenterology 47:468-472 (2000); and Pourgholami et al., Anticancer Res. 20:723-727 (2000)). Second, hVdup1 is decreased in HTLV-1 cell lines, while overexpression of hVdup1 suppresses their growth. Human Vdup1 is also frequently lost during tumor progression and cell transformation. Third, hVdup1 was found to be up-regulated by drug treatment in breast cancer cell lines that induced growth inhibition, cell cycle arrest, and apoptosis (Huang et al., Mol. Med. 6:849-866 (2000)). Fourth, coexpression of mVdup1 was shown to compete with both apoptosis signal-regulating kinase 1 (ASK-1) and the antiapoptotic proliferation-associated gene (PAG, also known as peroxiredoxin) for binding to TRX. Furthermore, when exposed to oxidative stress, NIH 3T3 cells overexpressing mVdup1 had elevated apoptotic cell death and decreased cell proliferation as compared to controls (Junn et al., 2000, supra). Thus, mVdup1 may function as a redox sensitive tumor suppressor by inhibiting TRX activity and competing with TRX-ASK1 and TRX-PAG binding, making cells more susceptible to growth inhibition in response to stress. Taken together, Vdup1, an inhibitor of TRX, may have an antitumorigenic effect in certain types of tumors.

From the use of a partial mVdup1, it was shown that residues 134-395 are involved in TRX binding and inhibition (Junn et al., 2000, supra). Since the mutant Vdup1 present in strain HcB-19 contains a nonsense mutation corresponding to amino acid 97, the truncated protein product will be missing these crucial amino acids. Thus, the HYPLIP1 nonsense mutation in Vdup1 likely results in misregulation of murine TRX. The Vdup1 nonsense mutation in HcB-19 animals may cause an increase in hepatic carcinoma formation and/or progression by affecting the TRX pathway, either through the general redox state of the cell or by modulating other functions of TRX, such as interaction with ASK-1 and peroxiredoxin.

Recently, DRH1, a novel protein with 41% identity to Vdup1, was demonstrated to be frequently down-regulated in expression in human hepatocellular carcinoma (29/35 tumors, 83%) (Yamamoto et al., Clin. Cancer Res. 7:297-303 (2001)). The DRH1 protein, like Vdup1, is located in the cytoplasm. Down-regulation of DRH1 expression was found to be closely associated with later events in hepatocarcinogenesis, particularly in metastasis and vascular invasion (Yamamoto et al., 2001, supra).

Mice and Mouse Husbandry. The development of the recombinant congenic mutant mouse strain HcB-19/Dem was described previously (Castellani et al., 1998, supra and Demant et al., 1986, supra). HcB-19 backcross animals were obtained by crossing with (HcB-19×CAST/Ei)F2 animals. All mice were housed in groups of five or less animals per cage and maintained on a 12 hour light-dark cycle at an ambient temperature of 23° C. They were allowed ad libitum access to water and standard Purina Rodent Chow containing 4.5% fat (Ralston-Purina Co.).

Analysis of Hepatic Tumors. Animals were sacrificed under isofluorane anesthesia and the liver removed and grossly examined for the presence of hepatic tumors. If a tumor was observed, a section was taken and preserved in 10% formalin, with the remainder of the tumor immediately frozen on dry ice to preserve for expression analysis. Tissue sections were imbedded in paraffin and stained with hematoxylin and eosin for histopathology. A portion of normal liver tissue from the same animals was used as controls, as well as liver tissue from unaffected animals.

TABLE 3
Hepatic Tumor Occurrence in HcB-19 and C3H Mice

| Tumor | Number of | | Average | Min. | Max. | |
|---|---|---|---|---|---|---|
| Presence | Animals | Strain | Age | Age | Age | Sex |
| None | 12 | C3H | 293 ± 32 | | | |
| None | 12 | C3H | 308 ± 31 | | | |
| Total: | 0/24 (0%) | C3H | 300 ± 22 | | | |
| None | 13 | HcB | 336 ± 46 | 235 | 631 | F |
| None | 10 | HcB | 345 ± 23 | 251 | 463 | F |
| Yes | 14 | HcB | 453 ± 45 | 251 | 717 | M |
| Yes | 6 | HcB | 356 ± 20 | 285 | 390 | F |
| Total: | 20/43 (47%) | HcB | 379 + 22 | 235 | 717 | M/F |

The tumor presence or absence is indicated for the total number of animals observed from each strain, either the control strain C3H/DiSnA (C3H) or the mutant HcB-19/Dem (HcB) strain containing the Vdup1 nonsense mutation.
The average + s.e.m. age, minimum age (Min.), and maximum age (Max.) in days is given for each group.

TABLE 4
Hepatic Tumor Occurrence in (HcB-19 X CAST/Ei)F2 Backcross
Animals Heterozygous or Homozygous for the HYPLIP1 Nonsense
Mutation in Vdup1

| Tumor | Number of | | Average | Min. | Max. | |
|---|---|---|---|---|---|---|
| Presence | Animals | Genotype | Age | Age | Age | Sex |
| None | 8 | +/− | 384 ± 40 | | | |
| None | 26 | +/− | 378 ± 19 | | | |
| Yes | 2 | +/− | 441 | 441 | 441 | M |
| Total: | 2/36 (6%) | +/− | 383 ± 16 | | | |
| None | 23 | −/− | 395 ± 24 | 202 | 609 | M |
| None | 45 | −/− | 414 ± 14 | | | |
| Yes | 16 | −/− | 492 ± 28 | 334 | 635 | M |
| Yes | 10 | −/− | 526 ± 17 | 403 | 583 | F |
| Total: | 26/94 (28%) | −/− | 435 + 11 | 202 | 635 | M/F |

The tumor presence or absence is indicated for the total number of animals observed from each genotype. Abbreviations:
−/−, homozygous for the HYPLIP1 nonsense mutation in Vdup1;
+/−, heterozygous;
M, male;
F, female.
The average + s.e.m. age, minimum age (Min.), and maximum age (Max.) in days is given for each group.

Although the foregoing invention has been described in some detail by way of illustration and example for purposes of clarity of understanding, it will be obvious that certain changes and modifications may be practiced within the scope of the appended claims.

All publications, patents, web sites are herein incorporated by reference in their entirety to the same extent as if each individual publication, patent or web site was specifically and individually indicated to be incorporated by reference in its entirety.

1. An isolated polynucleotide comprising a polynucleotide sequence selected from the group consisting of:
(a) a sequence variation of SEQ ID NO: 1, wherein said variation is associated with a lipid disorder;
(b) a complementary sequence of (a);
(c) a polynucleotide sequence having at least 65% sequence identity to sequence of (a); and
(d) a complementary sequence of (c).

2. An isolated polynucleotide comprising a sequence variation of SEQ ID NO: 2 or its complementary sequence, wherein said variation is associated with a lipid disorder.

3. An isolated polynucleotide comprising a polynucleotide sequence selected from the group consisting of:
(a) a sequence variation of SEQ ID NO: 4, wherein said variation is associated with a lipid disorder;
(b) a complementary sequence of (a);
(c) a polynucleotide sequence having at least 65% sequence identity to sequence of (a); and
(d) a complementary sequence of (c).

4. The polynucleotide of claim 1 or 3 wherein said variation is a mutation.

5. The polynucleotide of claim 1 or 3 wherein said variation is a polymorphism.

6. An isolated polypeptide comprising an amino acid sequence selected from the group consisting of:
(a) a variant form of SEQ ID NO: 3, wherein said variant form is associated with a lipid disorder; and
(b) an amino acid sequence having at least 65% sequence identity to sequence of (a).

7. An isolated polypeptide comprising an amino acid sequence selected from the group consisting of:
(a) a variant form of SEQ ID NO: 5, wherein said variant form is associated with a lipid disorder; and
(b) an amino acid sequence having at least 65% sequence identity to sequence of (a).

8. An isolated polynucleotide having at least 12 contiguous nucleotides of the polynucleotides of claim 1 or 3 wherein said 12 contiguous nucleotides span said variation position.

9. An isolated polypeptide having at least four contiguous amino acids of the polypeptides of claim 6 or 7 wherein said four contiguous amino acids span said variant position.

10. A polynucleotide specific for the HYPLIP1 locus wherein said polynucleotide hybridizes, under stringent conditions, to at least 12 contiguous nucleotides of the polynucleotide of claim 1 or 2.

11. The polynucleotide according to claim 10 wherein said polynucleotide is selected from the group consisting of SEQ ID NOs: 23-406.

12. A polynucleotide specific for the FCHL1 locus wherein said polynucleotide that hybridizes, under stringent conditions, to at least 12 contiguous nucleotides of the polynucleotide of claim 3.

13. The polynucleotide of claim 12 wherein said polynucleotide is selected from the group consisting of SEQ ID NOs: 6-22.

14. A kit for the detection of the FCHL1 locus comprising a polynucleotide of claim 12 and instructions relating to detection.

15. An isolated antibody which is immunoreactive to the polypeptide of claim 6 or 7.

16. A method for analyzing a biomolecule in a sample, wherein said method comprising:
(a) altering HYPLIP1 or FCHL1 activity in a sample; and
(b) measuring the concentration of a lipid-associated biomolecule.

17. A method for analyzing a polynucleotide in a sample comprising the steps of:
(a) contacting a polynucleotide in a sample with a probe wherein said probe hybridizes to the polynucleotides of claim 1 or 3 to form a hybridization complex; and
(b) detecting the hybridization complex.

18. A method for analyzing the expression of FCHL1 comprising the steps of (a) contacting a sample with a probe wherein said probe comprises the polynucleotide of claim 12; and
(b) detecting the expression of FCHL1 mRNA transcript in said sample.

19. A method for identifying susceptibility to a lipid disorder which comprises comparing the nucleotide sequence of the suspected FCHL1 allele with a wild-type FCHL1 nucleotide sequence, wherein said difference between the suspected allele and the wild-type sequence identifies a sequence variation of FCHL1 nucleotide sequence.

20. An expression vector comprising the polynucleotide of claim 1 or 3.

21. A host cell comprising the expression vector of claim 20.

22. A method of producing a polypeptide comprising culturing the host cells of claim 21 and recovering the polypeptide from the host cell.

23. A pharmaceutical composition comprising
(a) the polynucleotide of claim 3, the polypeptide of claim 7, or an isolated antibody which is immunoreactive to the polypeptide of claim 7; and
(b) a suitable pharmaceutical carrier.

24. A method for treating or preventing a lipid disorder associated with expression of FCHL1, wherein said method comprising administering to a subject an effective amount of the pharmaceutical composition of claim 23.
